(19) United States
(12) Patent Application Publication
(10) Pub. No.: US 2005/0203043 A1
(43) Pub. Date: Sep. 15, 2005

(54) Identification of toxic nucleotide sequences

(75) Inventors: Yuriy Fedorov, Superior, CO (US); Jon Karpilow, Boulder, CO (US); Anastasia Khvorova, Boulder, CO (US)

Correspondence Address: KALOW & SPRINGUT LLP, 488 MADISON AVENUE, 19TH FLOOR, NEW YORK, NY, 10022, US

(73) Assignee: DHARMACON, INC., Lafayette, CO (US)

(21) Appl. No.: 11/040,553

(22) Filed: Jan. 21, 2005

Related U.S. Application Data
(60) Provisional application No. 60/538,874, filed on Jan. 23, 2004.
(60) Provisional application No. 60/548,285, filed on Feb. 27, 2004.
(60) Provisional application No. 60/642,059, filed on Jan. 7, 2005.

(51) Int. Cl. C12Q 1/68; G06F 19/00; G01N 33/48; G01N 33/50; C07H 21/04; C07H 21/02

(52) U.S. Cl. 514/44; 435/455; 435/6; 536/23.1; 702/20

(57) ABSTRACT

Toxic nucleic acid sequences and methods for identifying, using, and screening libraries for them, including in silico screening, are provided. Compositions of the invention comprise unimolecular and double stranded polynucleotides comprising at least one toxicity region. Toxic sequences of the invention include A/G UUU A/G/U, G/C AAA G/C, and/or GCCA, NUUU, wherein N is any nucleotide, or complements thereof. The invention also provides a method of inducing a toxic response in a cell, comprising introducing into the cell a unimolecular or double stranded polynucleotide comprising at least one toxicity region comprising a sequence selected from the group consisting of A/G UUU A/G/U, G/C AAA G/C, GAAT, and GCCA, NUUU, wherein N is any nucleotide, or a complement of any of the foregoing, wherein said unimolecular or double stranded polynucleotide is at least 5 base pairs long and is comprises a sense and antisense region that are at least substantially complementary.

### CROSS REFERENCE TO RELATED APPLICATIONS

This application is a nonprovisional of, and claims the benefit of the filing date of, U.S. Provisional Patent Application Ser. No. 60/538,874, filed Jan. 23, 2004; U.S. Provisional Patent Application Ser. No. 60/548,285, filed Feb. 27, 2004; and U.S. Provisional Patent Application Ser. No. ______, filed Jan. 7, 2005, as attorney docket no. 13591PA2, entitled “Identification of Toxic Sequences,” with Express Mail Label No. EV279582915US; each of the above-mentioned applications is incorporated herein by reference.

### FIELD OF INVENTION

The present invention relates to toxic sequences in polynucleotides in general, including polynucleotides comprising RNA, and RNAs that can participate in RNA-induced gene silencing.

### BACKGROUND OF THE INVENTION

A variety of molecules, including those that are proteinaceous, nucleic acid, lipid, or carbohydrate in nature, can induce cytotoxic effects (see, for instance, Gururaja, T. et al. (2003) “Cellular interacting proteins of functional screen-derived antiproliferative and cytotoxic peptides discovered using shotgun peptide sequencing” Chem. Biol. 10(10):927-37). Knowledge of the cellular specificity and mechanism of action of such molecules is valuable from both a research and therapeutic perspective. For instance, studies of anthrax toxins identified these factors as initiators of caspase-dependent apoptosis. Similarly, studies of toxin B from Clostridium difficile aided in the elucidation of the function of the Rho family of proteins in cell signaling (see, for instance, Schmidt, M. et al. (1996) “Inhibition of receptor signaling to phospholipase D by Clostridium difficile toxin B. Role of Rho proteins.” J. Biol. Chem. 271(5):2422-6).

Although proteins are the focus of most current drug discovery efforts, research has recently begun that aims to exploit nucleic acids as novel agents and targets for pharmaceutical development. Toxic RNA, DNA, or RNA-DNA hybrid sequences (either single stranded or double stranded) can be valuable as therapeutic agents or co-agents that can be used in collaboration with other molecules to, e.g, sensitize target cells to undergo apoptosis or necrosis. The targets of these molecules can be diverse. In some instances, the target(s) of a toxic oligonucleotide is nucleic acid in nature (DNA or RNA) and the mechanism of action is related to the relative degree of homology that the toxic sequence has for a specific target molecule (e.g., antisense and RNA interference (RNAi), Layery, K. S. et al. (2003) “Antisense and RNAi: powerful tools in drug target discovery and validation” Curr. Opin. Drug Discov. Devel. 6(4):561-9; Provost et al., (2002) “Ribonuclease Activity and RNA Binding of Recombinant Human Dicer” E.M.B.O. J., 21(21): 5864-5874; Tabara et al. (2002) “The dsRNA Binding Protein RDE-4 Interacts with RDE-1, DCR-1 and a DexH-box Helicase to Direct RNAi in C. elegans” Cell 109(7):861-71; Ketting et al. (2001) “Dicer Functions in RNA Interference and in Synthesis of Small RNA Involved in Developmental Timing in C. elegans” Genes and Development 20:2654-9; Martinez et al. (2002) “Single-Stranded Antisense siRNAs Guide Target RNA Cleavage in RNAi” Cell 110(5):563; Hutvagner & Zamore (2002) “A microRNA in a multiple-turnover RNAi enzyme complex” Science 297:2056). Alternatively, oligonucleotides can target protein sequences. In these instances, the mechanism of toxicity can involve the molecule assuming a three-dimensional structure that is capable of blocking a critical function of the target protein. Alternatively, the sequence of the oligonucleotide can be recognized by the protein and, for instance, eliminate the ability of that target from participating in critical cellular reactions.

There is a need in the art for knowledge of oligonucleotide sequences that induce such toxicity in cells. In some instances, such sequences need to be identified so that their use can be avoided. In other instances, toxic sequences can be identified and used to benefit in a variety of applications, including pharmaceutical applications. The following disclosure addresses these needs.

### SUMMARY OF THE INVENTION

The present invention is directed to toxic polynucleotide sequences and methods of using and identifying them.

According to a first embodiment, the present invention provides a unimolecular polynucleotide, comprising at least one toxicity region comprising a sequence selected from the group consisting of GUUU (SEQ. ID NO.1), AGCA (SEQ. ID NO.2), GCAC (SEQ. ID NO.3), CUGG (SEQ. ID NO.4), AGAC (SEQ. ID NO.5), UGGC (SEQ. ID NO.6), NUUU (SEQ. ID NO.7), wherein N is any nucleotide, or a complement of any of the foregoing, wherein said unimolecular polynucleotide is capable of forming an intramolecular duplex of 5 or more base pairs, and wherein said duplex comprises a sense region and an antisense region that are at least substantially complementary.

According to a second embodiment, the invention provides a double stranded polynucleotide, comprising at least one toxicity region comprising a sequence selected from the group consisting of GUUU (SEQ. ID NO.1), AGCA (SEQ. ID NO.2), GCAC (SEQ. ID NO.3), CUGG, (SEQ. ID NO.4) AGAC (SEQ. ID NO.5), UGGC, (SEQ. ID NO.6) NUUU, (SEQ. ID NO.7) wherein N is any nucleotide, or a complement of any of the foregoing, wherein said double stranded polynucleotide is capable of forming a duplex of 5 or more base pairs, and wherein said duplex comprises a sense strand and an antisense strand that are at least substantially complementary.

According to a third embodiment, the invention provides a composition for inducing a toxic response in a cell, comprising a nucleotide sequence GUUU (SEQ. ID NO.1), AGCA (SEQ. ID NO.2), GCAC (SEQ. ID NO.3), CUGG (SEQ. ID NO.4), AGAC (SEQ. ID NO.5), UGGC (SEQ. ID NO.6), NUUU (SEQ. ID NO.7), wherein N is any nucleotide, or a complement of any of the foregoing, wherein said nucleotide sequence comprises a duplex region that is at least 5 base pairs in length, and wherein said duplex region comprises at least two regions that are at least substantially complementary.

According to a fourth embodiment, the invention provides a method of inducing a toxic response in a cell, said method comprising introducing into the cell a unimolecular polynucleotide or a double stranded polynucleotide, wherein said unimolecular polynucleotide or double stranded polynucleotide comprises at least one toxicity region comprising a sequence selected from the group consisting of GUUU (SEQ. ID NO.1), AGCA (SEQ. ID NO.2), GCAC (SEQ. ID NO.3), CUGG (SEQ. ID NO.4), AGAC (SEQ. ID NO.5), UGGC (SEQ. ID NO.6), NUUU (SEQ. ID NO.7), wherein N is any nucleotide, or a complement of any of the foregoing, wherein said unimolecular polynucleotide or double stranded polynucleotide comprises a duplex region of 5 or more base pairs, and wherein said unimolecular polynucleotide or double stranded polynucleotide comprises a sense region and an antisense region that are at least substantially complementary.

According to a fifth embodiment, the invention provides a method for screening a library of nucleic acids for a toxicity region, comprising screening a database containing nucleic acid sequences and identifying those sequences that contain toxic motifs.

According to a sixth embodiment, the invention provides a transfection control method, comprising: (a) transfecting a first group of cells with one or more polynucleotides or double-stranded polynucleotides; (b) transfecting a second group of cells with a duplex RNA, wherein said duplex RNA comprises at least one toxicity region comprising a sequence selected from the group consisting of GUUU (SEQ. ID NO.1), AGCA (SEQ. ID NO.2), GCAC (SEQ. ID NO.3), CUGG (SEQ. ID NO.4), AGAC (SEQ. ID NO.5), UGGC (SEQ. ID NO.6), NUUU (SEQ. ID NO.7), wherein N is any nucleotide, or a complement of any of the foregoing, wherein said duplex RNA is 5 or more base pairs in length, and wherein said duplex RNA comprises a sense region and an antisense region that are at least substantially complementary, and wherein said first and said second cells are transfected under similar conditions; (c) maintaining said first and said second groups of cells under conditions sufficient for cell growth; and (d) determining the level of cell viability in said second group of cells.

For a better understanding of the present invention together with other and further advantages and embodiments, reference is made to the following description taken in conjunction with the examples, the scope of which is set forth in the appended claims.

### BRIEF DESCRIPTION OF THE FIGURES

The preferred embodiments of the invention have been chosen for purposes of illustration and description but are not intended to restrict the scope of the invention in any way. The preferred embodiments of certain aspects of the invention are shown in the accompanying figures, wherein:

FIGS. 1a illustrates survival rates of HeLa cells transfected with siRNA directed against raf1, mek1 (MAP2K1), mek2 (MAP2K2), mapk1, mapk3, PI3k-Ca, PI3k-Cb, Bcl2, Bcl13, SRD5A1, SRD5A2, or AR. Lipofectamine 2000 was used to introduce the duplexes into the cells. siRNA concentrations were 10 nanomolar. Four siRNAs were tested against each gene. Cell survival rate was measured 72 hours post-transfection using Alamar Blue. 1b illustrates the ratio of toxic and non-toxic siRNA in a second set of sequences. 1c illustrates the distribution of toxic and non-toxic siRNAs across a walk covering a portion of the DBI gene. Gray bars show toxicity. Black bars show gene silencing.

FIG. 2a illustrates the sequences of toxic and non-toxic duplexes and the frequency with which these motifs are found in toxic and non-toxic populations identified in FIG. 1a. Underscored sequences represent the motifs that are observed in toxic molecules of this study of a limited set of molecules.

FIGS. 2b-1 to 2b-3 illustrates the frequency of finding toxic siRNA in groups that have the UUU/AAA motif, (SEQ. ID NO.8/9) the GCCA/UGGC motif, (SEQ. ID NO.10/11) or no motif at all. Black bars represent toxic siRNA. Gray bars represent non-toxic siRNA.

FIG. 2c illustrates the distribution of toxic and non-toxic siRNA in a large collection (>290) siRNA used in a statistical analysis to identify toxic motifs. Black bars represent toxic siRNA, gray bars represent non-toxic siRNA.

FIG. 2d illustrates the relative frequency of various motifs in the RISC-entering strand and non-entering strand of toxic and non-toxic siRNA.

FIG. 3 illustrates the concentration dependence of the m23 (MAP2K2-3) toxic siRNA sequence.

FIG. 4 illustrates the lack of correlation between silencing and toxicity of the MEK1 (MAP2K1) and MEK2 (MAP2K2) sequences.

FIG. 5a illustrates the design of Ago2 (eIF2C2) knockdown experiments; 5b—iillustrate the results of control experiments. Knockdown of Ago2 prevents subsequent attempts to knockdown a reporter gene (EGFP) using EGFP-targeting siRNA; 5j illustrates that toxic siRNA are not toxic in an Ago2⁻ cell; 5K illustrates that when toxic siRNA (19mers) are reduced to 17mers, toxicity is attenuated; 5l illustrates that addition of chemical modifications that eliminate off-target effects, attenuates toxicity.

FIG. 6 is a phase contrast photograph of cells undergoing apoptosis after being transfected with a toxic duplex (sense sequence, 5′ GGACAUUUGUGUACUCACU). (SEQ. ID NO.12)

FIG. 7 illustrates the results of staining cells with Hoechst 33342. A and B are controls (untransfected and Lipofectamine 2000 treated, respectively). C and D show cultures that are transfected with two different duplexes that contain toxic sequences GCUACUAUCUGAUUUACUG (SEQ. ID NO.13) or GGACAUUUGUGUACUCACU, respectively (SEQ. ID NO.12)). Circled cells represent those undergoing apoptosis.

FIGS. 8a-e illustrate the level of cell death (apoptosis) induced in HeLa, PC3, MCF7, LnCAP, and BxPC3 cell lines using non-toxic and toxic siRNA. Abbreviations and additional sequences used in these experiments include: MAP2K2-1=m21, MAP2K2-3=m23, SRD5A2-1=s21, SRD5A2-3=s23, Luciferase (Luc) dx 1-2 (l 12, 5′-UUUGUGGACGAAGUACCGA, sense (SEQ. ID NO.14)), Luc dx 1-4=l 14 (5′ UGUUUGUGGACGAAGUACC, sense (SEQ. ID NO.15)), Luc dx 2-3 (l 23, 5′ GAGUUGUGUUUGUGGACGA, sense (SEQ. ID NO.16)), PPIB dx10=cyclophilin 10=c10 (5′-UUGGCUACAAAAACAGCAA, sense (SEQ. ID NO.17)), PPIB dx 5=cyclophilin 5=c5 (5′ AAAACAGUGGAUAAUUUUG, sense (SEQ. ID NO.18)), PPIB dx 8=cyclophilin 8=c8 (5′ GGAUAAUUUUGUGGCCUUA, sense (SEQ. ID NO.19)).

FIG. 9 illustrates the ability of toxic sequences to sensitize cells to H₂O₂. Non-toxic sequences include Mek2-4 (MAP2K2-4, m24), SRD5A2-1, and PPIB dx 5. Toxic Sequences include Mek2-3 (MAP2K2-3, m23), SRD5A2-3, and PPIB dx 8. (see legend for FIG. 8 and Table 1 for sequences).

FIG. 10 illustrates the toxicity of two non-specific siRNAs that contain the GCCA motif (SEQ. ID NO.10) (n6, 5′ ACUCUAGCGCCAUCGUGCC and n7, 5′ ACUCUAUCGCCAGCGUGAC) (SEQ. ID NO.20 AND 21) and a third, non-specific siRNA that does not contain the GCCA motif (SEQ. ID NO.10) (n8,5′-ACUCUAUCUGCACGCUGAC (SEQ. ID NO.22)).

### DEFINITIONS

Unless stated otherwise, the following terms and phrases include the meanings provided below:

Agent that Stresses a Cell

The phrase “agent that stresses a cell” includes any agent known in the art, or that comes to be known in the art, that can induce—on its own or in combination with any of the compositions or methods of the present invention—a toxic or stress response in a cell, including but not limited to apoptosis and cell death. Agents that induce stress can be chemical in nature (e.g., H₂O₂), physical in nature (e.g., less than optimal temperatures), biological (e.g., viral infection), and more. Alternatively, cells can be stressed by the absence of essential agents such as growth factors (e.g., insulin), O₂, and other factors. Further, cellular stress can be measured in a variety of ways including but not limited to monitoring cell viability (cell death), cell doubling times, cell morphology, and expression of genes or gene families including those related to hypoxia responses, heat shock responses, cell cycle regulation, the interferon response pathway and others.

Antisense Region

The phrase “antisense region” refers to a sequence of nucleotides in a polynucleotide that is at least substantially complementary to a sense region in the same polynucleotide (if the polynucleotide is a unimolecular polynucleotide having both a sense and antisense sequence, wherein the sense and antisense sequences are capable of annealing by reason of the polynucleotide forming intramolecular interactions such as, for example, a hairpin structure), or in a different polynucleotide (in the case of a double stranded polynucleotide that comprises two separate strands, one bearing a sense sequence and one bearing an antisense sequence, wherein the sense and antisense sequences are capable of annealing by reason of the two strands undergoing an intermolecular interaction to form, for example, a duplex).

Antisense Strand

The phrase “antisense strand” as used herein, refers to a polynucleotide that is at least substantially or 100% complementary to a target nucleic acid of interest. An antisense strand may comprise a polynucleotide that is RNA, DNA or chimeric RNA/DNA. For example, an antisense strand may be complementary, in whole or in part, to a molecule of messenger RNA, an RNA sequence that is not mRNA (e.g., tRNA, rRNA and hnRNA) or a sequence of DNA that is either coding or non-coding.

Complementary

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated.

Perfect complementarity or 100% complementarity refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. “Substantial complementarity” refers to polynucleotide strands exhibiting 79% or greater complementarity, excluding regions of the polynucleotide strands, such as overhangs, that are selected so as to be noncomplementary. (“Substantial similarity” refers to polynucleotide strands exhibiting 79% or greater similarity, excluding regions of the polynucleotide strands, such as overhangs, that are selected so as not to be similar.) Thus, for example, two polynucleotides of 29 nucleotide units each, wherein each comprises a di-dT at the 3′ terminus such that the duplex region spans 27 bases, and wherein 26 of the 27 bases of the duplex region on each strand are complementary, are substantially complementary since they are 96.3% complementary when excluding the di-dT overhangs.

Deoxynucleotide

The term “deoxynucleotide” refers to a nucleotide or polynucleotide lacking a hydroxyl group (OH group) at the 2′ and/or 3′ position of a sugar moiety. Instead it has a hydrogen bonded to the 2′ and/or 3′ carbon. Within an RNA molecule that comprises one or more deoxynucleotides, “deoxynucleotide” refers to the lack of an OH group at the 2′ position of the sugar moiety, having instead a hydrogen bonded directly to the 2′ carbon.

Deoxyribonucleotide

The terms “deoxyribonucleotide” and “DNA” refer to a nucleotide or polynucleotide comprising at least one sugar moiety that has an H, rather than an OH, at its 2′ and/or 3′ position.

Duplex Region

The phrase “duplex region” or “duplex RNA” refers to the region in two complementary or at least substantially complementary polynucleotides that form base pairs with one another, either by Watson-Crick base pairing or any other manner that allows for a stabilized duplex between polynucleotide strands that are complementary or at least substantially complementary. For example, a polynucleotide strand having 21 nucleotide units can base pair with another polynucleotide of 21 nucleotide units, yet only 19 bases on each strand are complementary or at least substantially complementary, such that the “duplex region” has 19 base pairs. The remaining bases may, for example, exist as 5′ and 3′ overhangs. Further, within the duplex region, 100% complementarity is not required; substantial complementarity is allowable within a duplex region. Substantial complementarity refers to 79% or greater complementarity. For example, a mismatch in a duplex region consisting of 19 base pairs results in 94.7% complementarity, rendering the duplex region substantially complementary. Duplex regions or duplex RNA can be the result of the pairing of two separate strands. Alternatively, duplexes regions can be the result of pairing of two complementary regions existing within a unimolecular sequence.

Essential Gene

The term “essential gene” refers to a specific nucleotide coding sequence whose expression product is vital for cell survival. An essential gene may encode any one of a variety of different polypeptides whose function is indispensable for cell viability. Consequently, inactivation of an essential gene generally results in cell death and/or cell stress. Inactivation may occur through a variety of mechanisms occurring at the DNA, mRNA, and protein levels. A genetic variation, for example, such as a single nucleotide polymorphism (SNP), occurring within the DNA coding sequence itself may alter, diminish, or eliminate the biological function of the resulting expression product. Alternatively, an essential gene may be inactivated at the mRNA level through an siRNA-mediated RNA interference pathway.

Gene Silencing

The phrase “gene silencing” refers to a process by which the expression of a specific gene product is lessened or attenuated. Gene silencing can take place by a variety of pathways. Unless specified otherwise, as used herein, gene silencing refers to decreases in gene product expression that results from RNA interference (RNAi), a defined, though partially characterized pathway whereby small inhibitory RNA (siRNA) act in concert with host proteins (e.g., the RNA induced silencing complex, RISC, or the RNA-induced Initiation of Transcriptional Gene Silencing, RITS) to degrade messenger RNA (mRNA) in a sequence-dependent fashion or affect gene expression by other pathways or mechanisms, including but not limited to epigenetic mechanisms such as DNA and/or histone methylation. The level of gene silencing can be measured by a variety of means, including, but not limited to, measurement of transcript levels by Northern Blot Analysis, B-DNA techniques, transcription-sensitive reporter constructs, expression profiling (e.g., DNA chips), and related technologies. Alternatively, the level of silencing can be measured by assessing the level of the protein encoded by a specific gene. This can be accomplished by performing a number of studies including Western Analysis, measuring the levels of expression of a reporter protein that has, for example, fluorescent properties (e.g., GFP) or enzymatic activity (e.g. alkaline phosphatases), or other procedures.

Nucleotide

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

Modified bases refer to nucleotide bases such as, for example, adenine, guanine, cytosine, thymine, uracil, xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

The term nucleotide is also meant to include what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group.

Further, the term nucleotide also includes those species that have a detectable label, such as for example a radioactive or fluorescent moiety, or mass label attached to the nucleotide. Nucleotides can also be detected by their inherent mass.

Off-Target

The term “off-target” and the phrase “off-target effects” refer to any instance in which an siRNA or shRNA directed against a given target causes an unintended effect by interacting either directly or indirectly with another mRNA sequence, a DNA sequence or a cellular protein or other moiety. For example, an “off-target effect” may occur when there is a simultaneous degradation of other transcripts due to partial homology or complementarity between that other transcript and the sense and/or antisense strand of the siRNA or shRNA

Overhang

The term “overhang” refers to terminal non-base pairing nucleotide(s) resulting from one strand extending beyond the terminus of the complementary strand to which the first strand forms a doubled stranded polynucleotide. One or both of two polynucleotides that are capable of forming a duplex through hydrogen bonding of base pairs may have a 5′ and/or 3′ end that extends beyond the 3′ and/or 5′ end of complementarity shared by the two polynucleotides. The single-stranded region extending beyond the 3′ and/or 5′ end of the duplex is referred to as an overhang.

Pharmaceutically Acceptable Carrier

The phrase “pharmaceutically acceptable carrier” includes compositions that facilitate the introduction of dsRNA, dsDNA, or dsRNA/DNA hybrids into a cell and includes but is not limited to solvents or dispersants, coatings, anti-infective agents, isotonic agents, and agents that mediate absorption time or release of the inventive polynucleotides and double stranded polynucleotides.

Polynucleotide

The term “polynucleotide” refers to polymers of nucleotides, and includes but is not limited to DNA, RNA, DNA/RNA hybrids including polynucleotide chains of regularly and irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. A polynucleotide comprises two or more nucleotides.

Polyribonucleotide

The term “polyribonucleotide” refers to a polynucleotide comprising two or more modified or unmodified ribonucleotides and/or their analogs. The term “polyribonucleotide” is used interchangeably with the term “oligoribonucleotide.”

Rational Design

The term “rational design” describes a set of criteria, developed at Dharmacon, Inc. that allow the identification of functional, highly functional, and hyperfunctional siRNAs. The set of criteria used to identify these siRNA have been incorporated into an algorithm that can be applied to any gene, regardless of the gene's origin. The criteria associated with rational design have been described in U.S. Provisional Patent Application Ser. No. 60/426,137, filed Nov. 14, 2002, entitled “Combinatorial Pooling Approach for siRNA Induced Gene Silencing and Methods for Selecting siRNA”; U.S. Provisional Patent Application Ser. No. 60/502,050, filed Sep. 10, 2003, entitled “Methods for Selecting siRNA”; U.S. patent application Ser. No. 10/714,333, filed Nov. 14, 2003, entitled “Functional and Hyperfunctional siRNA,” each of which is incorporated by reference herein.

Ribonucleotide and Ribonucleic Acid

The term “ribonucleotide” and the phrase “ribonucleic acid” (RNA), refer to a modified or unmodified nucleotide or polynucleotide comprising at least one ribonucleotide unit. A ribonucleotide unit comprises a hydroxyl group attached to the 2′ position of a ribosyl moiety that has a nitrogenous base attached in N-glycosidic linkage at the 1′ position of a ribosyl moiety, and a moiety that either allows for linkage to another nucleotide or precludes linkage.

RISC-Entering Strand

The term “RISC-entering strand” refers to the strand of a siRNA that preferably enters RISC. Determination of which strand preferably enters RISC can be made based on thermodynamic calculations that take into consideration end stability of the duplex as well as the average internal stability profile (AISP) of the entire siRNA.

RNA Interference and RNAi

The phrase “RNA interference” and the term “RNAi” are synonymous and refer to the process by which a polynucleotide or double stranded polynucleotide comprising at least one ribonucleotide unit exerts an effect on a biological process. The process includes but is not limited to gene silencing by degrading mRNA, interactions with tRNA, rRNA, hnRNA, cDNA and genomic DNA, as well as methylation of DNA with ancillary proteins.

Sense Region

The phrase “sense region” refers to a sequence of nucleotides in a polynucleotide that is at least substantially complementary to an antisense region in the same polynucleotide (if the polynucleotide is a unimolecular polynucleotide having both a sense and antisense sequence, wherein the sense and antisense sequences are capable of annealing by reason of the polynucleotide forming intramolecular interactions such as, for example, a hairpin structure), or in a different polynucleotide (in the case of a double stranded polynucleotide that comprises two separate strands, one bearing a sense sequence and one bearing an antisense sequence, wherein the sense and antisense sequences are capable of annealing by reason of the two strands undergoing an intermolecular interaction to form, for example, a duplex).

Sense Strand

The phrase “sense strand” refers to a polynucleotide that has the same nucleotide sequence, in whole or in part, as a target nucleic acid such as a messenger RNA or a sequence of DNA.

siRNA

The term “siRNA” refers to small inhibitory RNA duplexes that induce the RNA interference (RNAi) pathway. These molecules can vary in length (typically between 18-30 base pair) and contain varying degrees of complementarity to their target mRNA in the antisense strand. Some, but not all siRNA have unpaired, overhanging bases on the 5′ or 3′ end of the sense strand and/or the antisense strand.

An siRNA molecule can be bimolecular, comprising separate sense and antisense strands annealed through non-covalent interaction, or can be unimolecular, as when sense and antisense strands comprise regions of a hairpin structure that comprises a loop structure and, optionally, a stem region and/or terminal structure. Thus, a short hairpin RNA (shRNA) is a species of the genus siRNA.

Stressing a Cell

The phrase “stressing a cell” or “stress a cell,” includes placing a cell under conditions that are identified as being less than optimal for growth. Agents that induce stress can be chemical in nature (e.g., H₂O₂), physical in nature (e.g., less than optimal temperatures), biological (e.g., viral infection), and others. Alternatively, cells can be stressed by the absence of needed agents such as growth factors, O₂, and other factors. Stressing a cell also includes inducing cell death by apoptosis or other means. Cellular stress can be measured in a variety of ways and includes monitoring cell viability (cell death), cell doubling times, cell morphology, and expression of genes or gene families including those related to hypoxia responses, heat shock responses, cell cycle regulation, the interferon response pathway and others. Further, stressing a cell or population of cells can make said cells more susceptible to secondary reagents, such as toxic agents. Methods for identifying toxicity regions are disclosed herein.

Target

The term “target” is used in a variety of different contexts herein and is defined by the context in which it is used. “Target mRNA” refers to a messenger RNA to which a given siRNA can be directed against. “Target sequence” and “target site” refer to a sequence within the mRNA to which the sense strand of an siRNA shows varying degrees of homology and the antisense strand exhibits varying degrees of complementarity. The term “siRNA target” can refer to the gene, mRNA, or protein against which an siRNA is directed. Similarly “target silencing” can refer to the state of a gene, or the corresponding mRNA or protein. The phrase “target of a toxic sequence” refers to the nucleotide or protein to which a given siRNA or shRNA interacts with to induce a state of stress in the cell. These differences in context reflect the mechanism(s) by which toxic sequences can exert their toxic effects on a cell.

Toxicity Region

The phrase “toxicity region” refers to a nucleotide sequence in a polynucleotide that confers upon the polynucleotide the ability to stress a cell.

Toxic Response

The phrase “toxic response” includes cellular responses to stress. Such responses can be identified by any suitable method in the art for measuring the effect of toxins on cells, or for measuring cell viability. Suitable methods include, for example, those methods used in the art to measure cell death (e.g., apoptosis), DNA replication, cell metabolism, and induction of one or more pathways associated with response to cell stress including hypoxia responses, heat shock responses, cell cycle regulation, the interferon response pathway and others.

Transfection

The term “transfection” refers to a process by which agents are introduced into a cell. The list of agents that can be transfected is large and includes, but is not limited to, siRNA, sense and/or anti-sense sequences, DNA encoding one or more genes and organized into an expression plasmid, proteins, protein fragments, and more. There are multiple methods for transfecting agents into a cell including, but not limited to, electroporation, CaPO₄-based transfections, DEAE-dextran-based transfections, lipid-based transfections, molecular-conjugate-based transfections (e.g., polylysine-DNA conjugates), microinjection and others.

All nucleotide sequences are written from 5′ to 3′, that is, the 5′ end of the sequence on the left, and the 3′ end of the sequence on the right.

### PREFERRED EMBODIMENTS

The present invention will now be described in connection with preferred embodiments. These embodiments are presented to aid in an understanding of the present invention and are not intended, and should not be construed to limit the invention in any way. All alternatives, modifications and equivalents that may become apparent to those of ordinary skill upon reading this disclosure are included within the spirit and scope of the present invention.

This disclosure is not a primer on compositions and methods for performing RNA interference. Basic concepts known to those skilled in the art have not been set forth in detail.

### SUMMARY

The present invention includes a collection of novel toxic motifs and methods of use of said sequences. The toxic motifs include: GUUU (SEQ. ID NO.1), AGCA (SEQ. ID NO.2), GCAC (SEQ. ID NO.3), CUGG (SEQ. ID NO.4), AGAC (SEQ. ID NO.5), UGGC (SEQ. ID NO.6), NUUU (SEQ. ID NO.7), wherein N is any nucleotide, and complements of any of the foregoing.

Knowledge of said motifs can be valuable in a variety of fields. For example, in instances pertaining to the use of RNAi as a method of gene function analysis, unintentional introduction of an siRNA comprising a toxic motif into a cell can result in cell death and misinterpretation of the essential nature of said gene. Thus, under these conditions, knowledge of toxic motifs allows researches to better design and/or select for siRNA that lack such undesirable sequences.

Alternatively, for example, where siRNAs are being used as therapeutic reagents and are being designed to, for example, induce cell death in a population of cells such as, for example, diseased cells, siRNAs directed against a given target that contain toxic sequences are more likely to induce cell death than siRNAs that do not contain toxic sequences. siRNAs containing one or more toxic sequences can be used individually, or can be combined with one or more additional therapeutic agents to treat a given disease. In the latter case, duplex RNA carrying one or more toxic motifs can be used to sensitize the target, such as, for example, a diseased cell, to a second reagent.

Double stranded RNA carrying toxic motifs are also valuable as controls in experiments that require transfection of polynucleotides such as, for example, DNA and/or RNA, into cultured cells. In this instance, the level of cell death induced by a duplex carrying a toxic motif can be used to assess the success of the transfection procedure, thus minimizing the costs associated with processing samples and assessing data derived from failed transfections.

### EMBODIMENTS

According to a first embodiment, the present invention provides a unimolecular polynucleotide, comprising at least one toxicity region comprising a sequence selected from the group consisting of GUUU (SEQ. ID NO.1), AGCA (SEQ. ID NO.2), GCAC (SEQ. ID NO.3), CUGG (SEQ. ID NO.4), AGAC (SEQ. ID NO.5), UGGC (SEQ. ID NO.6), NUUU (SEQ. ID NO.7), wherein N is any nucleotide, or a complement of any of the foregoing, wherein said unimolecular polynucleotide is capable of forming an intramolecular duplex of 5 or more base pairs, and wherein said duplex comprises a sense region and an antisense region that are at least substantially complementary.

The unimolecular polynucleotide can have a sense region that comprises at least one toxicity region, and/or an antisense region that comprises at least one toxicity region. As experiments described in the Examples clearly demonstrate regarding the strand preference of the toxic motif, in the cases of NUUU (SEQ. ID NO.7), or UGGC motifs (SEQ. ID NO.6), preferably the motifs are present in the RISC-entering strand when the desired effect is to induce cellular toxicity.

The at least one toxicity region can be located in a variety of positions within the duplex.

Preferably, the unimolecular polynucleotide has an antisense region that comprises from 19 to 40 bases. The unimolecular polynucleotide can comprise a loop region, a stem region, and/or a terminal region. Preferably, the sense region and antisense region are more than substantially complementary over the range of base pairs, and more preferably 100% complementary over this range. Preferably the polynucleotide is RNA.

In the case of a unimolecular polynucleotide, the order or sequence in which each component appears in the sequence can vary, being either 5′ S-loop-AS or 5′-AS-loop-S. The preferred arrangement is 5′ AS-loop-S. The preferred length of both the sense and antisense regions is 19-40 nucleotides in length. Preferably, the unimolecular polynucleotide comprises a loop, wherein the loop preferably comprises 4-20 nucleotides. Moreover, the terminal region of the unimolecular polynucleotide can be blunt, or have overhangs of 1-5 nucleotides on the 3′ or 5′ end. Preferably, the overhangs are on the 3′ end of the molecule. Preferably, the 5′ end of the molecule has a phosphate group on the 5′ carbon.

In some cases, where strong toxicity is desired, the region of the duplex comprises RNA greater than 40 base pairs. Such duplexes (>40 base pairs) are also capable of inducing cell death by the interferon response pathway. Thus, in cases where toxic sequences are associated with longer (>40 base pair) duplexes, cell death is induced by at least two mechanisms: (1) the action of the toxic motif, and (2) the induction of the interferon response. More preferably, in cases where strong toxicity is desired, the toxic sequence is associated with a long (>40 base-pair) RNA duplex that also targets an essential gene or essential non-coding RNA (e.g., an miRNA) by the RNAi pathway. In these cases, cell death is the result of three separate actions: (1) the action of the toxic motif; (2) the induction of the interferon response pathway by the long double-stranded RNA; and (3) the loss of function of an essential gene or function.

According to a second embodiment, the invention provides a double stranded polynucleotide, comprising at least one toxicity region comprising a sequence selected from the group consisting of GUUU (SEQ. ID NO.1), AGCA (SEQ. ID NO.2), GCAC (SEQ. ID NO.3), CUGG (SEQ. ID NO.4), AGAC (SEQ. ID NO.5), UGGC (SEQ. ID NO.6), NUUU (SEQ. ID NO.7), wherein N is any nucleotide, or a complement of any of the foregoing, wherein said double stranded polynucleotide is capable of forming a duplex of 5 or more base pairs, and wherein said duplex comprises a sense strand and an antisense strand that are at least substantially complementary.

In one preferred embodiment, the double stranded polynucleotide comprises a sense strand comprising at least one toxicity region. In another preferred embodiment, the double stranded polynucleotide comprises an antisense strand comprising at least one toxicity region.

The toxic motif can be located in a variety of positions within the duplex.

In cases where the double stranded polynucleotide is an siRNA, preferably the siRNA comprises from 1940 base pairs, or from 19-23 base pairs, exclusive of overhangs. Preferably, the sense strands and antisense strands are at least substantially complementary over the range of base pairs, and more preferably 100% complementary over this range. Preferably the polynucleotide is RNA.

The double stranded polynucleotide may also contain overhangs at either the 5′ or 3′ end of either the sense strand or the antisense strand. However, if there are any overhangs, they are preferably on the 3′ end of the sense strand and/or the antisense strand. Additionally, any overhangs are preferably six or fewer bases in length, more preferably two or fewer bases in length. Most preferably, there are either no overhangs, or overhangs of two bases on one or both of the sense strand and antisense strand.

The first 5′ terminal antisense nucleotide and/or the first 5′ terminal sense nucleotide may or may not be modified with a phosphate group attached to the 5′ carbon of the sugar moiety of the nucleotide. If there is a phosphate group, preferably there is only one phosphate group.

In some cases, where strong toxicity is desired, the region of the duplex comprises RNA and is greater than 40 base pairs. Such duplexes (>40 base pairs) are capable of inducing cell death by the interferon response pathway. Thus, in cases where toxic sequences are associated with longer (>40 base pair) RNA duplexes, cell death is induced by at least two mechanisms: (1) the action of the toxic motif, and (2) the induction of the interferon response. Even more preferably, in cases where strong toxicity is desired, the toxic sequence is associated with a long (>40 base-pair) RNA duplex that also targets an essential gene by the RNAi pathway. In these cases, cell death is the result of three separate actions: (1) the action of the toxic motif; (2) the induction of the interferon response pathway by the long double-stranded RNA; and (3) the loss of function of an essential gene by the RNAi pathway.

According to a third embodiment, the invention provides a composition for inducing a toxic response in a cell, comprising a nucleotide sequence GUUU (SEQ. ID NO.1), AGCA (SEQ. ID NO.2), GCAC (SEQ. ID NO.3), CUGG (SEQ. ID NO.4), AGAC (SEQ. ID NO.5), UGGC (SEQ. ID NO.6), NUUU (SEQ. ID NO.7), wherein N is any nucleotide, or a complement of any of the foregoing, wherein said nucleotide sequence comprises a duplex region that is at least 5 base pairs in length, and wherein said duplex region is comprises at least two regions that are at least substantially complementary. The duplex region can be unimolecular such as, for example, a hairpin structure described in Embodiment 1, or can comprise two separate strands (such as the molecule(s) described in Embodiment 2. Preferably, the composition is an RNA or an siRNA.

According to a fourth embodiment, the invention provides a method of inducing a toxic response in a cell, comprising introducing into the cell a unimolecular polynucleotide or a double stranded polynucleotide, wherein said unimolecular polynucleotide or double stranded polynucleotide comprises at least one toxicity region comprising a sequence selected from the group consisting of GUUU (SEQ. ID NO.1), AGCA (SEQ. ID NO.2), GCAC (SEQ. ID NO.3), CUGG (SEQ. ID NO.4), AGAC (SEQ. ID NO.5), UGGC (SEQ. ID NO.6), NUUU (SEQ. ID NO.7), wherein N is any nucleotide, or a complement of any of the foregoing, wherein said unimolecular polynucleotide or double stranded polynucleotide comprises a duplex region of 5 or more base pairs, and wherein said unimolecular polynucleotide or double stranded polynucleotide comprises a sense region and an antisense region that are at least substantially complementary.

Toxic motifs can be located in a variety of positions within the duplex.

In one preferred embodiment, the method further comprises exposing the cell to at least one agent or condition that stresses the cell, in addition to the cited toxicity sequence(s). The at least one agent or condition that stresses the cell can be any agent or condition known in the art that can stress a cell. Agents can include, for example, chemotherapeutic agents such as cisplatin, proleukin, Campath, 9-cis-retinoic acid, cyclophosphamide, dacarbazine, as well as other siRNA/antisense agents that are directed against specific targets. In cases where the agent that stresses the cell is a peroxide, the peroxide is preferably hydrogen peroxide.

Inducing a toxic response in a cell includes sensitizing a cell to stress. In this manner, a cell can be sensitized to the actions of other agents, stressors, or environments, such as, for example, those that induce apoptosis or cell death. Any agent or environment known in the art, or that comes to be known, that can induce stress, apoptosis, or cell death can be used in conjunction with the compositions and methods of the invention. Thus, for instance, conditions that deplete the local environment of, for example, necessary nutrients, growth factors, oxygen (e.g., hypoxia) or other necessary factors, are included herein. Such conditions can include, for example, radiation.

According to a fifth embodiment, the invention provides a method for screening a library of nucleic acids for a toxicity region, comprising screening a database containing nucleic acid sequences and identifying those sequences that contain toxic motifs. Screening can be accomplished visually or with the aid of a computer (i.e., in silico) to identify toxic motifs. A search of a library of sequences in one or more data files can be performed to identify library members that comprise one or more toxic regions. Thus, for example, the siRNA library described in U.S. patent application Ser. No. 10/714,333, filed Nov. 14, 2003, entitled “Functional and Hyperfunctional siRNA,” which contains roughly 1.6 million sequences, can be screened. Members of this library are rationally designed to silence specific gene targets from the human genome and can be useful in, for example, dissecting the roles of genes of known and unknown function. The presence of toxic sequences can be of inherent value, or detrimental, depending upon the use of such sequences. In cases where such agents are intended for therapeutic purposes, designed to, for example, kill diseased cells, presence of a toxic sequence within the siRNA can be valuable. Under these conditions, it would be desirable to screen through the library to identify sequences that contain toxic motifs. In contrast, in instances where the siRNAs are intended to be used for, for example, gene function analysis, presence of a toxic motif within the siRNA is undesirable. Under these conditions, it would be beneficial to screen through the contents of this library and identify/eliminate any library members that contain said toxic regions. Such screens may be performed with or without a computer program that allows for the cross-referencing of the toxic sequence with each of the library's siRNA. In the case where computer programs are used, the program may, for example, be accessible from a local terminal or personal computer, over an internal network or over the Internet. The computer program that may be used may be developed in any computer language that is known to be useful for scoring nucleotide sequences, or it may be developed with the assistance of a commercially available product.

Knowledge of toxic sequences will also enable modification of random and/or random-biased nucleic acid library synthesis strategies so as to minimize (or maximize) the possibility of introducing toxic sequences into one or more library members. Nucleic acid library design can be an important factor in obtaining, for example, functional siRNA, ribozymes, and/or antisense molecules that are capable of inducing high levels of, for example, (1) gene silencing, or (2) cell death. The occurrence of toxic sequences within such agents can be detrimental or beneficial, depending upon the intended use of such sequences. For example, in a research setting where a single, non-essential gene or gene product is under investigation, knockdown studies that employ siRNA that contain toxic sequences may induce phenotypes that mislead an investigator into believing that the gene is essential. Thus, under these circumstances, foreknowledge of a toxic sequence will enable the applicants to employ biases during, for example, random siRNA library design procedures that would minimize the chances of introducing toxic sequences into library members.

In another example, existing algorithms for designing polynucleotides such as, for example, siRNAs, or libraries of siRNAs, can be improved using the compositions and methods of the present invention. In one non-limiting example, algorithms for the selection of siRNAs of varying functionalities can be improved by incorporating into such algorithms, scripts (i.e., software code) that eliminate all sequences that contain the toxic motifs disclosed herein. Alternatively, algorithms can be modified to specifically select siRNAs that contain one or more toxic sequences.

According to a sixth embodiment, the invention provides a transfection control method, comprising: (a) transfecting a first group of cells with one or more polynucleotides or double-stranded polynucleotides; (b) transfecting a second group of cells with a duplex RNA, wherein said duplex RNA comprises at least one toxicity region comprising a sequence selected from the group consisting of GUUU (SEQ. ID NO.1), AGCA (SEQ. ID NO.2), GCAC (SEQ. ID NO.3), CUGG (SEQ. ID NO.4), AGAC (SEQ. ID NO.5), UGGC (SEQ. ID NO.6), NUUU (SEQ. ID NO.7), wherein N is any nucleotide, or a complement of any of the foregoing, wherein said duplex RNA is 5 or more base pairs in length, and wherein said duplex RNA comprises a sense region and an antisense region that are at least substantially complementary, and wherein said first and said second cells are transfected under similar conditions; (c) maintaining said first and said second groups of cells under conditions sufficient for cell growth; and (d) determining the level of cell viability in said second group of cells. Preferably, the RNA duplex is greater than 40 base pairs in length. More preferably, the RNA duplex is greater than 64 base pairs in length. In a preferred embodiment, the RNA duplex is an siRNA and targets an essential gene.

Applicants have found that of the toxic sequences described herein, the sequences GUUU (SEQ. ID NO.1) or UGGC (SEQ. ID NO.6), when present together or independently in a duplex of 17 basepairs or less, do not induce a toxic response, or induce a toxic effect. However, a duplex of 19 basepairs or more that comprises the sequences GUUU (SEQ. ID NO.1) or UGGC (SEQ. ID NO.6), independently or together, does induce a toxic effect. Thus, preferably, in order for an siRNA having the sequences GUUU (SEQ. ID NO.1) or UGGC (SEQ. ID NO.6), independently or together, to induce a toxic effect, the siRNA is preferably at least 18 basepairs in length.

The methods and compositions described herein can be used, for example, in the design and uses of molecules for control purposes. For example, toxic sequences can be incorporated into the design of molecules intended for use as, for example, transfection controls. Control transfections that use the methods and compositions described herein can be run alongside experimental transfections to verify, for example, the efficiency of transfection and the quality of the transfection reagents. In another non-limiting example, transfection controls in accordance with the methods and compositions described herein can help identify optimum conditions for transfection of any cell lines.

Typical RNAi silencing experiments benefit from controls that allow the experimenter to assess the fraction of cells that have been successfully transfected with a given test siRNA. In some instances, these controls can target specific genes and transfection efficiency can be determined by assessing the level of silencing of the targeted gene (e.g., by Northern or Western blot). In other instances, transfection efficiency can be assessed by labeling an siRNA with, for example, a fluorescent label, and subsequently identifying the fraction of cells that fluoresce using fluorescence microscopy or fluorescence-activated cell sorting (FACS). However, measuring transfection efficiency by assessing the level of silencing of a control gene is labor intensive and expensive. Similarly, instruments for measuring cellular fluorescence are expensive, and many laboratories may lack the resources and/or training necessary to access and operate such instruments. The present invention offers a novel alternative, whereby transfection efficiency is more easily measured using toxic polynucleotide sequences.

In one non-limiting example, the level of transfection in a given experiment can be assessed by measuring the level of cell death induced by transfection of a sequence that induces cell death. For instance, cells in control wells can be transfected with a duplex RNA that comprises one or more of the toxic sequences disclosed herein, for example, GUUU (SEQ. ID NO.1), AGCA (SEQ. ID NO.2), GCAC (SEQ. ID NO.3), CUGG (SEQ. ID NO.4), AGAC (SEQ. ID NO.5), UGGC (SEQ. ID NO.6), NUUU (SEQ. ID NO.7), wherein N is any nucleotide, motifs. Subsequently, after a period of, for example, 24, 48, or 72 hours the number of dead and/or dying cells can be determined using any suitable assay, including but not limited to Alamar Blue assays. If the total number of living cells represents only a small fraction (for instance 10%) of those present in wells that were not transfected with the duplex RNA containing the toxic motif, then this would indicate that 90% of the cells were successfully transfected.

In cases where duplex RNA comprising a toxic motif is used as a positive control for transfection efficiency, the number of motifs can be greater than one and the size of the duplex can vary greatly. In general, larger RNA duplexes that carry a toxic motif and target an essential gene via the RNAi pathway are preferred, since such molecules induce cytotoxicity by at least three mechanisms: (1) toxicity due to the toxic motif; (2) toxicity due to introduction of a large double stranded RNA that induces the interferon response; and (3) toxicity due to the introduction of a double stranded RNA that also targets an essential gene. Where duplex RNA comprising a toxic motif is used as a positive control for transfection efficiency, the size of the duplex carrying the toxic motif can be between 19-30 base pairs. More preferably, the size of the duplex carrying the toxic motif is between 19 and 42 base pairs. Even more preferably, the size of duplex carrying the toxic motif is between 19 and 64 base pairs. Even more preferably, the size of the RNA duplexes containing the toxic motif is greater than 64 base pairs. Most preferably, the RNA duplex carries one or more toxic motifs, is greater than 64 base pairs, and targets an essential gene by the RNAi pathway.

The methods of the embodiments of the invention are not limited by the cell type used, the methods of transfection, or the assay utilized to assess the cell stress, apoptosis, or cell death. Thus the present invention may use a diverse set of cell types, including primary cells, germ cell lines and somatic cell lines. The cells may be stem cells or differentiated cells. For example, the cell types may be embryonic cells, oocytes, sperm cells, adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes and cells of the endocrine or exocrine glands. Said cells can be plated at a variety of densities and. cultured in a variety of formats (e.g. 96- or 384-well plates). To induce a toxic response, cells are preferably plated at a high density (>90% confluency). More preferably, the cells are plated at a moderate density (65-90% confluency). Most preferably, the cells are plated at a low density (<50% confluency).

Many techniques are known in the art for introducing nucleic acids into cells. Any suitable technique can be used for introducing toxic sequences into cells. These techniques include, but are not limited to, electroporation, lipid-mediated transfection, chemical-mediated transfection, viral-mediated transduction and others. In the case of viral mediated expression of toxic sequences, vectors that are based on lentiviral or retroviral systems are preferred. Any method of introducing polynucleotides, or double stranded polynucleotides, that are known in the art, or that come to be known, can be used with the methods and compositions herein.

Many techniques for assessing cell stress, apoptosis, and cell death are known in the art. Any suitable technique can be used in combination with the toxic motifs reported in this document. These techniques include, but are not limited to, one or more kits that utilize dyes (e.g., Alamar Blue), antibodies (e.g., Apo2.7), enzymatic activities (e.g., caspases), gene expression (e.g., microarrays), or other parameters to quantitate the level or degree of cell death. Any method of assessing cell stress, apoptosis, or cell death that is known in the art, or that comes to be known, can be used with the methods and compositions herein.

Regarding each of the embodiments of the invention, the toxic sequence(s) or their complement(s) are not specifically designed to target a specific gene. As shown in Example 3, there is no necessary correlation between the toxicity of an siRNA carrying said motif, and target knockdown. Instead, toxic siRNA carrying the motif(s) of the invention appear to be acting by inducing off-target gene knockdown. Without wishing to be bound by any particular theory, the toxic effects associated with exposing a cell to a toxic sequence may be the result of the toxic sequence interacting with a specific and essential target in the cell. Without limitation, such a target may be proteinaceous in nature. Alternatively, and without limitation, the target of the toxic sequence can be nucleic acid, lipid, or carbohydrate in nature.

Any of the compositions and methods of the invention can be used in combination with other compositions and methods known in the art. For example, toxic sequences can be used in conjunction with therapeutic small molecules, other therapeutic nucleic acids, small peptides, proteins, lipids, combinations thereof, or other agents that alter or affect the functionality of one or more targets within the cell. Introduction of such agents can precede the introduction of a toxic sequence, follow the introduction of a toxic sequence, or be applied simultaneously with a toxic sequence. Thus, for instance, a toxic sequence can be delivered along with a second agent that is a unimolecular polynucleotide or double stranded polynucleotide, wherein the second agent recognizes and down regulates a transcript responsible for a specific disease (for example, a cancer). In this non-limiting example, conditions are selected so as to introduce either agent individually, inducing minimal levels of cell death. Yet the combination of both the toxic sequence and the second agent that is a unimolecular polynucleotide or double stranded polynucleotide is sufficiently toxic to induce cell death in diseased cells.

Potential benefits can be realized by including knowledge of toxic sequences in, for example, siRNA design. For instance, when trying to kill or disable cells that contain, for example, a disease-related SNP (single nucleotide polymorphism), identification of an siRNA or antisense molecule that (in addition to recognizing the SNP) also contains a toxic sequence, would be beneficial. The methods and compositions of the invention can be useful in the design of therapeutics and therapies that employ agents comprising nucleic acids, including agents that comprise polynucleotides, to eliminate one or more cells or cell types related to a disease or an undesirable phenotype.

Another benefit of the methods and compositions of the invention is the ability to target certain cells, such as, for example, deleterious cells, for destruction. For example, the present invention may be used in RNA interference applications, wherein an siRNA having a toxic sequence is designed to be directed against a specific gene in a specific cell type. For these applications, an organism suspected of having a disease or disorder that is amenable to modulation by manipulation of a particular target nucleic acid of interest is treated by administering siRNA. The organism can be a mammal, such as, for example, a mouse, rat, sheep, cow, or human. Results of the siRNA treatment may be ameliorative, palliative, prophylactic, and/or diagnostic of a particular disease or disorder. Preferably, the siRNA is administered in a pharmaceutically acceptable manner with a pharmaceutically acceptable carrier or diluent.

Therapeutic applications of the present invention can be performed with a variety of therapeutic compositions and methods of administration. Pharmaceutically acceptable carriers and diluents are known to persons skilled in the art. Methods of administration to cells and organisms are also known to persons skilled in the art. Dosing regimens, for example, are known to depend on the severity and degree of responsiveness of the disease or disorder to be treated, with a course of treatment spanning from days to months, or until the desired effect on the disorder or disease state is achieved. Chronic administration of siRNAs may be required for lasting desired effects with some diseases or disorders. Suitable dosing regimens can be determined by, for example, administering varying amounts of one or more siRNAs in a pharmaceutically acceptable carrier or diluent, by a pharmaceutically acceptable delivery route, and amount of drug accumulated in the body of the recipient organism can be determined at various times following administration. Similarly, the desired effect (for example, degree of suppression of expression of a gene product or gene activity) can be measured at various times following administration of the siRNA, and this data can be correlated with other pharmacokinetic data, such as body or organ accumulation. Those of ordinary skill can determine optimum dosages, dosing regimens, and the like. Those of ordinary skill may employ EC₅₀ data from in vivo and in vitro animal models as guides for human studies.

Further, the polynucleotides can be administered in a cream or ointment topically, an oral preparation such as a capsule or tablet or suspension or solution, and the like. The route of administration may be intravenous, intramuscular, dermal, subdermal, cutaneous, subcutaneous, intranasal, oral, rectal, by eye drops, by tissue implantation of a device that releases the siRNA at an advantageous location, such as near an organ or tissue or cell type harboring a target nucleic acid of interest.

Still further, the present invention may be used in RNA interference applications, such as diagnostics, prophylactics, and therapeutics including use of the composition in the manufacture of a medicament in animals, preferably mammals, more preferably humans, in the treatment of diseases, or over or under expression of a target. Preferably, the disease or disorder is one that arises from the malfunction of one or more genes, the disease or disorder of which is relate to the expression of the gene product of the one or more genes. For example, it is widely recognized that certain cancers of the human breast are related to the malfunction of a protein expressed from a gene commonly known as the “bcl-2” gene. A medicament can be manufactured in accordance with the compositions and teachings of the present invention, employing one or more siRNAs directed against the bcl-2 gene, and optionally combined with a pharmaceutically acceptable carrier, diluent and/or adjuvant, which medicament can be used for the treatment of breast cancer. Applicants have established the use of the methods and compositions of the present invention in cellular models. Methods of delivery of polynucleotides to cells within animals, including humans, are well known in the art. Any delivery vehicle now known in the art, or that comes to be known, and has utility for introducing polynucleotides to animals, including humans, is expected to be useful in the manufacture of a medicament in accordance with the present invention, so long as the delivery vehicle is not incompatible with any modifications that may be present in a composition made according to the present invention. A delivery vehicle that is not compatible with a composition made according to the present invention is one that reduces the efficacy of the composition by greater than 95% as measured against efficacy in cell culture.

Animal models exist for many disorders, including, for example, cancers, diseases of the vascular system, inborn errors or metabolism, and the like. It is within ordinary skill in the art to administer nucleic acids to animals in dosing regimens to arrive at an optimal dosing regimen for a particular disease or disorder in an animal such as a mammal, for example, a mouse, rat or non-human primate. Once efficacy is established in the mammal by routine experimentation by one of ordinary skill, dosing regimens for the commencement of human trials can be arrived at based on data arrived at in such studies.

Dosages of medicaments manufactured in accordance with the present invention may vary from micrograms per kilogram to hundreds of milligrams per kilogram of a subject. As is known in the art, dosage will vary according to the mass of the mammal receiving the dose, the nature of the mammal receiving the dose, the severity of the disease or disorder, and the stability of the medicament in the serum of the subject, among other factors well known to persons of ordinary skill in the art.

The compositions and methods of the present invention can be employed with any suitable modifications known in the art, as long as the modifications do not substantially interfere with the efficacy of the methods or compositions of the invention. Substantial interference with the methods or compositions of the invention results when the modification(s) reduces the efficacy of the composition or method by greater than 90%, as compared to the efficacy of the composition or method in the absence of the modification. Many modifications are known in the art. Preferred modifications are disclosed in U.S. patent application Ser. No. 10/406,908, filed Apr. 2, 2003, entitled “Stabilized Polynucleotides for Use in RNA Interference,” and U.S. patent application Ser. No. 10/613,077, filed Jul. 1, 2003, entitled “Stabilized Polynucleotides for Use in RNA Interference,” each of which is incorporated by reference herein.

Having described the invention with a degree of particularity, examples will now be provided. These examples are not intended to and should not be construed to limit the scope of the claims in any way. Although the invention may be more readily understood through reference to the following examples, they are provided by way of illustration and are not intended to limit the present invention unless specified.

### EXAMPLES

The following examples are intended to explain the invention further.

General Procedures

Transfection

HEK293, HeLa, MCF7 and DU145 cell lines were obtained from ATCC (Manassas, Va.). Cells were grown at 37° C. in a humidified atmosphere with 5% CO₂ in cell line-specific media: HEK293-DMEM, 10% FBS (Invitrogen), HeLa, DMEM, 10% FBS, MCF7, MEM (Invitrogen), 10% FBS, PC3, RPMI, 10% FBS. All propagation media was supplemented with penicillin (100 U/ml) and streptomycin (100 ug/ml). For transfection experiments, cells were seeded at 5×10³ cells/well in 96 well plates 24 h before the experiment in antibiotic free media. The cell density described for these experiments was critical for observing siRNA-induced toxicity. Cells were transfected with siRNA (10 nM, 1 pmole/well) using Lipofectamine 2000 (0.1 ul/well, Invitrogen) according to manufacturer instruction. For gene expression analysis in HEK293 (LUC walk) cells were transfected as described earlier [Reynolds, 2004 #1081]. Presented graphs represent the average values obtained from three independent experiments, each performed in triplicate. Error bars represent standard deviation.

Cell Viability Assay

The survival of cells after treatment was determined by Alamar Blue (BioSource Int.) cytotoxicity assay according to manufacturers instructions. Briefly, 72 h (HeLa) or 144 h (MCF7, PC3) after transfection, 25 ul of Alamar Blue dye were added to wells containing cells in 100 ul of media. Cells were then incubated (0.5 hrs (HeLa) or 2 hrs (MCF7 and DU145) at 37° C. in a humidified atmosphere with 5% CO₂. The fluorescence was subsequently measured on a Perkin Elmer Wallac Vector2 1420 multi-label counter with excitation at 540 nm and emission at 590 nm. The data presented are an average of nine data points coming from three independent experiments performed on different days. For the purpose of this study, siRNAs were defined as toxic when the average from nine different experiments (taking into account standard deviations) showed cell viability below 75%.

Gene Expression Analysis

mRNA expression levels were determined using Quantigene® Kits (Genospectra, Fremont, Calif.) for branched DNA (bDNA) assay [Collins, 1997 #1018] according to manufacturer instructions. Level of mRNA of GAPDH (a housekeeping gene) was used as a reference.

Microscopy

Regular and fluorescent microscopy was used to obtain data on cellular and nuclei morphology. Live cells were stained with cell-permeable nuclear fluorescent dye Hoechst 33342 (2 ug/ml, 15 min at 37° C., Molecular Probes). Pictures were taken using Leica DML fluorescent microscope InSight CCD camera and SPOT 3.5 software.

Down-Regulation of Gene Silencing

HeLa cells were transfected (T1) with a pool of siRNA directed against eIF2C2 (1 pmole/well) or with a control siRNA (1 pmole/well). Cells were then replated at 48 hrs (5×10³ cells/well in 96 well plates) and co-transfected a second time (T2) with an EGFP expressing plasmid (20 ng/well) and (1) a control siRNA (0.1 pmole/well) or (2) EGFP siRNA (0.1 pmole/well). Twenty-four hours later cells were assayed for EGFP knockdown at mRNA level (branched DNA) and protein level (fluorescent microscopy). For toxicity analysis, cells were pre-transfected (T1) with control or eIF2C2 siRNA pool, replated and and then transfected with a set of toxic siRNAs.

### Example 1

### Identification of Toxic Sequences

To identify toxic sequences, HeLa cells were plated (5,000 cells/well) in a 96 well plate and cultured overnight. On the following day, cells (35-50% confluent) were transfected with one of 48 different siRNAs directed against one of 12 different targets (4 siRNA directed against each of the following genes: raf1, mek1 (MAP2K1), mek2 (MAP2K2), mapk1, mapk3, PI3k-Ca, PI3k-Cb, Bcl2, Bcl3, SRD5A1, SRD5A2, AR see Table 1). For transfection, siRNA concentrations were 10 nanomolar and the siRNA:lipid (Lipofectamine 2000) ratio was 1 picomole per 0.1 microgram. Twenty-four hours after transfection, cells received an additional 100 microliters of media (+serum). Subsequently, at t=72 hours, cell survival was assayed using the Alamar Blue cytotoxicity assay (Alamar Biosciences, Inc).

TABLE I

IDENTIFICATION OF TOXIC SEQUENCES

| | | sIRNA (SENSE | SEQ. ID |
|---|---|---|---|
| GENE | MOTIF | STRAND, 5′→3′) | NO. |
| MAPK1-1 | cuuug | ccaaagcucuggacuuauu | 23 |
| MAPK1-2 | | aaacagaucuuuacaagcu | 24 |
| MAPK1-3 | | caagaggauugaaguagaa | 25 |
| MAPK1-4 | auuuc | guacagggcuccagaaauu | 26 |
| MAPK3/ERK1-1 | | gaccggauguuaaccuuua | 27 |
| MAPK3/ERK1-2 | | agacugaccuguacaaguu | 28 |
| MAPK3/ERK1-3 | guuuc | gaaacuaccuacagucucu | 29 |
| MAPK3/ERK1-4 | | gcuacacgcaguugcagua | 30 |
| AR-1 | | ggaacucgaucguaucauu | 31 |
| AR-2 | | caagggagguuacaccaaa | 32 |
| AR-3 | | ucaaggaacucgaucguau | 33 |
| AR-4 | | gaaaugauugcacuauuga | 34 |
| SRD5A2-1 | | gcuacuaucugauuuacug | 35 |
| SRD5A2-2 | gcca | gcuaugcccuggccacuug | 36 |
| SRD5A2-3 | auuug | ggacauuuguguacucacu | 37 |
| SRD5A2-4 | | uugggugucuucuuauuua | 38 |
| SRD5A1-1 | gcca | gcagauacuugagccauug | 39 |
| SRD5A1-2 | auuudt/gcca | uaacugcagccaacuauuu | 40 |
| SRD5A1-3 | cuuuc/gcca | gaaagccuaugccacuguu | 41 |
| SRD5A1-4 | auuug | ccggaaauuugaagaguau | 42 |
| PIK3CA-1 | guuua | auguuuacuaccaaaugga | 43 |
| PIK3CA-2 | | aacuagaaguauguugcua | 44 |
| PIK3CA-3 | | aauggcuuugaaucuuugg | 45 |
| PIK3CA-4 | cuuuc | cugaagaaagcauugacua | 46 |
| PIK3CB-1 | | cgacaagacugccgagaga | 47 |
| PIK3CB-2 | | ucaagugucuccuaauaug | 48 |
| PIK3CB-3 | | ggauucaguuggagugauu | 49 |
| PTK3CB-4 | | uuucaagugucuccuaaua | 50 |
| BCL2-1 | | gggagauagugaugaagua | 51 |
| BCL2-2 | | gaaguacauccauuauaag | 52 |
| BCL2-3 | | guacgacaaccgggagaua | 53 |
| BCL2-4 | | agauagugaugaaguacau | 54 |
| BCL3-1 | gcca | gaacaccgagugccaagaa | 55 |
| BCL3-2 | cuuug | gagccuuacugccuuugua | 56 |
| BCL3-3 | cuuua | ggccggaggcgcuuuacua | 57 |
| BCL3-4 | | ucgacgcaguggacauuaa | 58 |
| RAF1-1 | | gcacggagauguugcagua | 59 |
| RAF1-2 | | gcaaagaacaucauccaua | 60 |
| RAF1-3 | | gacaugaaauccaacaaua | 61 |
| RAF1-4 | cuuug | caaagaacaucauccauag | 62 |
| MAP2K1-1 | | gcacauggauggagguucu | 63 |
| MAP2K1-2 | | gcagagagagcagauuuga | 64 |
| MAP2K1-3 | | gagguucucuggaucaagu | 65 |
| MAP2K1-4 | auuug | gagcagauuugaagcaacu | 66 |
| MAP2K2-1 | | caaagacgaugacuucgaa | 67 |
| NAP2K2-2 | | gaucagcauuugcauggaa | 68 |
| MAP2K2-3 | guuug | uccaggaguuugucaauaa | 69 |
| MAP2K2-4 | | ggaagcugauccaccuuga | 70 |

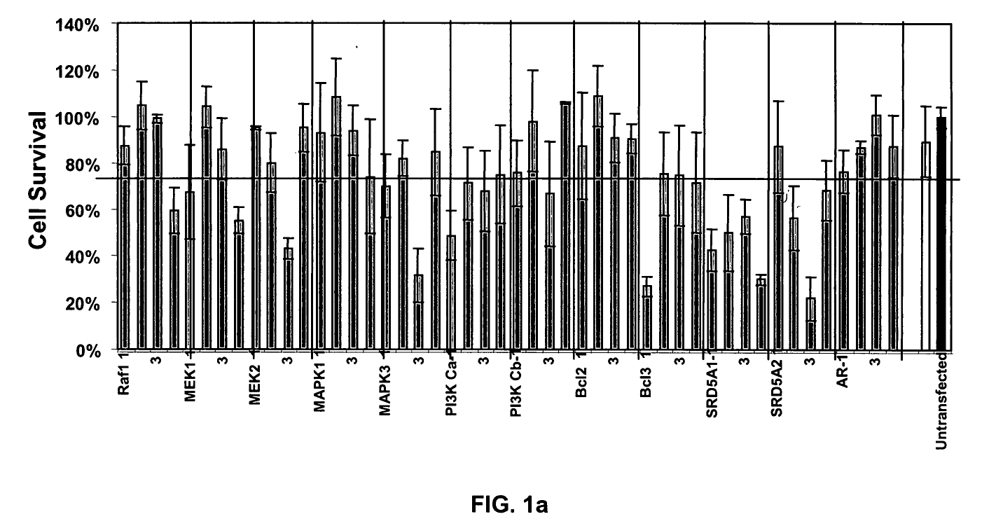

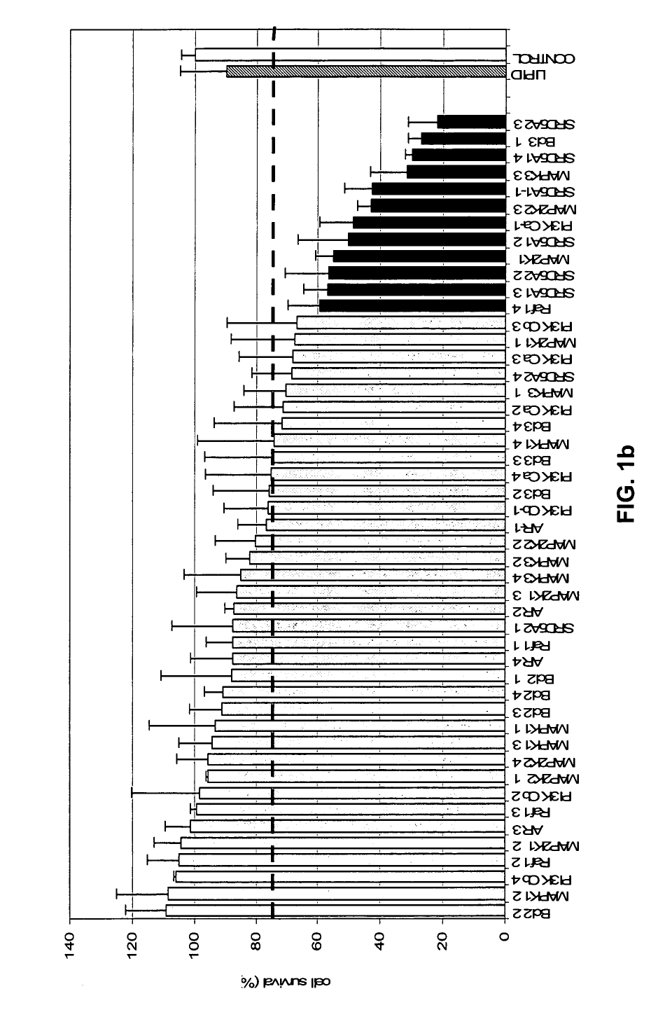

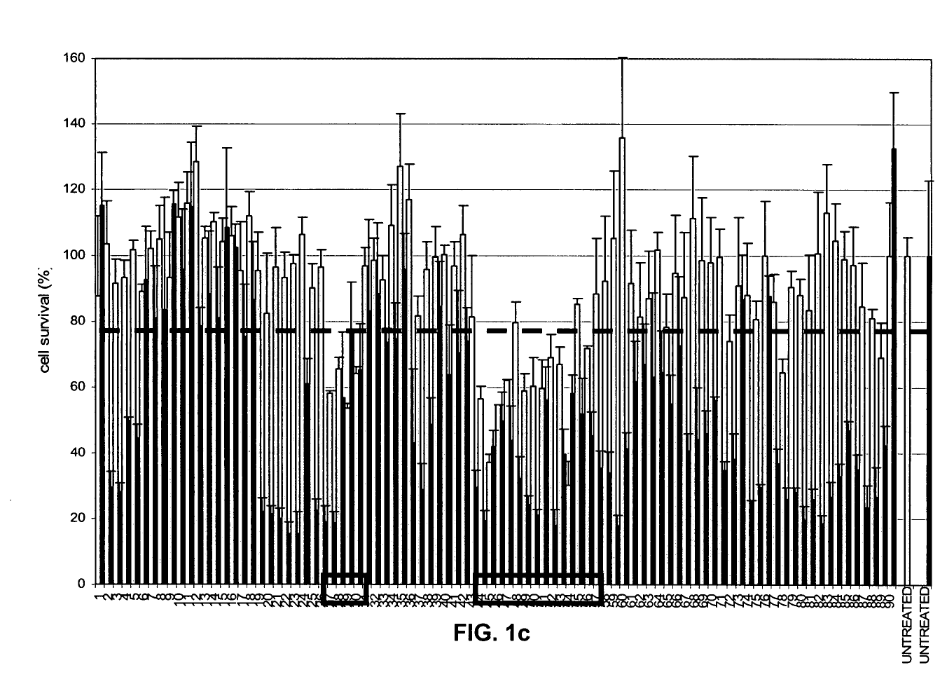

The results of these experiments, presented in FIG. 1a, show that each siRNA induced different levels of cell death. In several instances, a single siRNA out of the four directed against a single gene, induced extensive cell death while the remaining three duplexes induced lesser amounts of cytotoxicity. This result suggests that toxicity is unique to particular sequences and target independent. This conclusion was supported by examination of a second set of sequences targeting 12 different genes (FIG. 1b). The sequences and gene identities of this study includse:

TABLE II

SEQUENCES TARGETING DIFFERENT GENES

| | | | | |
|---|---|---|---|---|
| | | | | SEQ. |
| ACCESSION | GENE | SIRNA | SIRNA SEQUENCE | ID |
| No. | IDNTITY | NAME | (SENSE, 5′→3′) | NO. |

| | | | | |
|---|---|---|---|---|
| NM_000633 | Bc12 | Bc12 | gaaguacauccauuauaag | 71 |
| | | 2 | | |
| NM_002745 | MAPK1 | MAPK1 | aaacagaucuuuacaagcu | 72 |
| | | 2 | | |
| NM_006219 | PI3K Cb | PI3K Cb | uuucaagugucuccuaaua | 73 |
| | | 4 | | |
| NM_001654 | ARaf1 | Raf1 | gcaaagaacaucauccaua | 74 |
| | | 2 | | |
| NM_002755 | MAP2K1 | MAP2K1 | gcagagagagcagauuuga | 75 |
| | | 2 | | |
| NM_000044 | AR | AR | ucaaggaacucgaucguau | 76 |
| | | 3 | | |
| NM_001654 | ARaf1 | Raf1 | gacaugaaauccaacaaua | 77 |
| | | 3 | | |
| NM_006219 | PI3K Cb | PI3K Cb | ucaagugucuccuaauaug | 78 |
| | | 2 | | |
| NM_030662 | MAP2K2 | MAP2K2 | caaagacgaugacuucgaa | 79 |
| | | 1 | | |
| NM_030662 | MAP2K2 | MAP2K2 | ggaagcugauccaccuuga | 80 |
| | | 4 | | |
| NM_002745 | MAPK1 | MAPK1 | caagaggauugaaguagaa | 81 |
| | | 3 | | |
| NM_002745 | MAPK1 | MAPK1 | ccaaagcucuggacuuauu | 82 |
| | | 1 | | |
| NM_000633 | Bc12 | Bc12 | guacgacaaccgggagaua | 83 |
| | | 3 | | |
| NM_000633 | Bc12 | Bc12 | agauagugaugaaguacau | 84 |
| | | 4 | | |
| NM_000633 | Bc12 | Bc12 | gggagauagugaugaagua | 85 |
| | | 1 | | |
| NM_000044 | AR | AR | gaaaugauugcacuauuga | 86 |
| | | 4 | | |
| NM_001654 | ARaf1 | Raf1 | gcacggagauguugcagua | 87 |
| | | 1 | | |
| NM_000348 | SRD5A2 | SRD5A2 | gcuacuaucugauuuacug | 88 |
| | | 1 | | |
| NM_000044 | AR | AR | caagggagguuacaccaaa | 89 |
| | | 2 | | |
| NM_002755 | MAP2K1 | MAP2K1 | gagguucucuggaucaagu | 90 |
| | | 3 | | |
| NM_002746 | MAPK3 | MAPK3 | gcuacacgcaguugcagua | 91 |
| | | 4 | | |
| NM_002746 | MAPK3 | MAPK3 | agacugaccuguacaaguu | 92 |
| | | 2 | | |
| NM_030662 | MAP2K2 | MAP2K2 | gaucagcauuugcauggaa | 93 |
| | | 2 | | |
| NM_000044 | AR | AR- | ggaacucgaucguaucauu | 94 |
| | | 1 | | |
| NM_006219 | PI3K Cb | PI3K Cb- | cgacaagacugccgagaga | 95 |
| | | 1 | | |
| NM_005178 | Bc13 | Bc13 | gagccuuacugccuuugua | 96 |
| | | 2 | | |
| NM_006218 | PI3K Ca | PI3K Ca | cugaagaaagcauugacua | 97 |
| | | 4 | | |
| NM_005178 | Bc13 | Bc13 | ggccggaggcgcuuuacua | 98 |
| | | 3 | | |
| NM_002745 | MAPK1 | MAPK1 | guacagggcuccagaaauu | 99 |
| | | 4 | | |
| NM_005178 | Bc13 | Bc13 | ucgacgcaguggacauuaa | 100 |
| | | 4 | | |
| NM_006218 | PI3K Ca | PI3K Ca | aacuagaaguauguugcua | 101 |
| | | 2 | | |
| NM_002746 | MAPK3 | MAPK3 | gaccggauguuaaccuuua | 102 |
| | | 1 | | |
| NM_000348 | SRD5A2 | SRD5A2 | uugggugucuucuuauuua | 103 |
| | | 4 | | |
| NM_006218 | PI3K Ca | PI3K Ca | aauggcuuugaaucuuugg | 104 |
| | | 3 | | |
| NM_002755 | MAP2K1 | MAP2K1 | gcacauggauggagguucu | 105 |
| | | 1 | | |
| NM_006219 | PI3K Cb | PI3K Cb | ggauucaguuggagugauu | 106 |
| | | 3 | | |
| NM_001654 | ARaf1 | Raf1 | caaagaacaucauccauag | 107 |
| | | 4 | | |
| NM_001047 | SRD5A1 | SRD5A1 | gaaagccuaugccacuguu | 108 |
| | | 3 | | |
| NM_000348 | SRD5A2 | SRD5A2 | gcuaugcccuggccacuug | 109 |
| | | 2 | | |
| NM_002755 | MAP2K1 | MAP2K1 | gagcagauuugaagcaacu | 110 |
| | | 4 | | |
| NM_001047 | SRD5A1 | SRD5A1 | uaacugcagccaacuauuu | 111 |
| | | 2 | | |
| NM_006218 | PI3K Ca | PI3K Ca- | auguuuacuaccaaaugga | 112 |
| | | 1 | | |
| NM_030662 | MAP2K2 | MAP2K2 | uccaggaguuugucaauaa | 113 |
| | | 3 | | |
| NM_001047 | SRD5A1 | SRD5A1- | gcagauacuugagccauug | 114 |
| | | 1 | | |
| NM_002746 | MAPK3 | MAPK3 | gaaacuaccuacagucucu | 115 |
| | | 3 | | |
| NM_001047 | SRD5A1 | SRD5A1 | ccggaaauuugaagaguau | 116 |
| | | 4 | | |
| NM_005178 | Bc13 | Bc13 | gaacaccgagugccaagaa | 117 |
| | | 1 | | |
| NM_000348 | SRD5A2 | SRD5A2 | ggacauuuguguacucacu | 118 |
| | | 3 | | |

Again, the conclusion of this study was two-fold: 1) a fraction of siRNA induce toxicity and 2) the toxicity was target knockdown independent.

In a separate experiment 90 separate siRNAs covering a region of the DBI gene (NM_{—}020548, one base pair shift) were analyzed. The sequences used in this study are listed below.

TABLE III

DBI GENE STUDY SEQUENCES

| | | |
|---|---|---|
| SEQUENCE | | SEQ. ID |
| NUMBER | SEQUENCE (SENSE, 5′→3′) | NO. |

| | | |
|---|---|---|
| 1 | acgggcaaggccaaguggg | 119 |
| 2 | cgggcaaggccaaguggga | 120 |
| 3 | gggcaaggccaagugggau | 121 |
| 4 | ggcaaggccaagugggaug | 122 |
| 5 | gcaaggccaagugggaugc | 123 |
| 6 | caaggccaagugggaugcc | 124 |
| 7 | aaggccaagugggaugccu | 125 |
| 8 | aggccaagugggaugccug | 126 |
| 9 | ggccaagugggaugccugg | 127 |
| 10 | gccaagugggaugccugga | 128 |
| 11 | ccaagugggaugccuggaa | 129 |
| 12 | caagugggaugccuggaau | 130 |
| 13 | aagugggaugccuggaaug | 131 |
| 14 | agugggaugccuggaauga | 132 |
| 15 | gugggaugccuggaaugag | 133 |
| 16 | ugggaugccuggaaugagc | 134 |
| 17 | gggaugccuggaaugagcu | 135 |
| 18 | ggaugccuggaaugagcug | 136 |
| 19 | gaugccuggaaugagcuga | 137 |
| 20 | augccuggaaugagcugaa | 138 |
| 21 | ugccuggaaugagcugaaa | 139 |
| 22 | gccuggaaugagcugaaag | 140 |
| 23 | ccuggaaugagcugaaagg | 141 |
| 24 | cuggaaugagcugaaaggg | 142 |
| 25 | uggaaugagcugaaaggga | 143 |
| 26 | ggaaugagcugaaagggac | 144 |
| 27 | gaaugagcugaaagggacu | 145 |
| 28 | aaugagcugaaagggacuu | 146 |
| 29 | augagcugaaagggacuuc | 147 |
| 30 | ugagcugaaagggacuucc | 148 |
| 31 | gagcugaaagggacuucca | 149 |
| 32 | agcugaaagggacuuccaa | 150 |
| 33 | gcugaaagggacuuccaag | 151 |
| 34 | cugaaagggacuuccaagg | 152 |
| 35 | ugaaagggacuuccaagga | 153 |
| 36 | gaaagggacuuccaaggaa | 154 |
| 37 | aaagggacuuccaaggaag | 155 |
| 38 | aagggacuuccaaggaaga | 156 |
| 39 | agggacuuccaaggaagau | 157 |
| 40 | gggacuuccaaggaagaug | 158 |
| 41 | ggacuuccaaggaagaugc | 159 |
| 42 | gacuuccaaggaagaugcc | 160 |
| 43 | acuuccaaggaagaugcca | 161 |
| 44 | cuuccaaggaagaugccau | 162 |
| 45 | uuccaaggaagaugccaug | 163 |
| 46 | uccaaggaagaugccauga | 164 |
| 47 | ccaaggaagaugccaugaa | 165 |
| 48 | caaggaagaugccaugaaa | 166 |
| 49 | aaggaagaugccaugaaag | 167 |
| 50 | aggaagaugccaugaaagc | 168 |
| 51 | ggaagaugccaugaaagcu | 169 |
| 52 | gaagaugccaugaaagcuu | 170 |
| 53 | aagaugccaugaaagcuua | 171 |
| 54 | agaugccaugaaagcuuac | 172 |
| 55 | gaugccaugaaagcuuaca | 173 |
| 56 | augccaugaaagcuuacau | 174 |
| 57 | ugccaugaaagcuuacauc | 175 |
| 58 | gccaugaaagcuuacauca | 176 |
| 59 | ccaugaaagcuuacaucaa | 177 |
| 60 | caugaaagcuuacaucaac | 178 |
| 61 | augaaagcuuacaucaaca | 179 |
| 62 | ugaaagcuuacaucaacaa | 180 |
| 63 | gaaagcuuacaucaacaaa | 181 |
| 64 | aaagcuuacaucaacaaag | 182 |
| 65 | aagcuuacaucaacaaagu | 183 |
| 66 | agcuuacaucaacaaagua | 184 |
| 67 | gcuuacaucaacaaaguag | 185 |
| 68 | cuuacaucaacaaaguaga | 186 |
| 69 | uuacaucaacaaaguagaa | 187 |
| 70 | uacaucaacaaaguagaag | 188 |
| 71 | acaucaacaaaguagaaga | 189 |
| 72 | caucaacaaaguagaagag | 190 |
| 73 | aucaacaaaguagaagagc | 191 |
| 74 | ucaacaaaguagaagagcu | 192 |
| 75 | caacaaaguagaagagcua | 193 |
| 76 | aacaaaguagaagagcuaa | 194 |
| 77 | acaaaguagaagagcuaaa | 195 |
| 78 | caaaguagaagagcuaaag | 196 |
| 79 | aaaguagaagagcuaaaga | 197 |
| 80 | aaguagaagagcuaaagaa | 198 |
| 81 | aguagaagagcuaaagaaa | 199 |
| 82 | guagaagagcuaaagaaaa | 200 |
| 83 | uagaagagcuaaagaaaaa | 201 |
| 84 | agaagagcuaaagaaaaaa | 202 |
| 85 | gaagagcuaaagaaaaaau | 203 |
| 86 | aagagcuaaagaaaaaaua | 204 |
| 87 | agagcuaaagaaaaaauac | 205 |
| 88 | gagcuaaagaaaaaauacg | 206 |
| 89 | agcuaaagaaaaaauacgg | 207 |
| 90 | gcuaaagaaaaaauacggg | 208 |

Results of these studies are presented in FIG. 1c, and show (1) some, but not all, siRNA are toxic, (2) toxicity is unrelated to target knockdown, and (3) that toxic siRNA often cluster together (see black boxes). This last finding suggested that one or more motifs might be responsible for the observed toxicity.

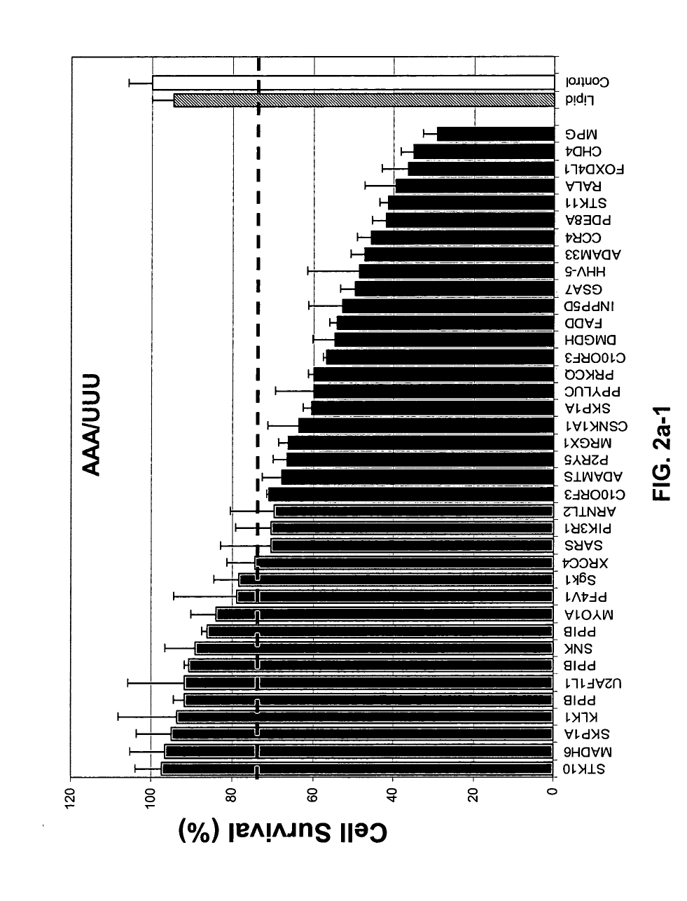

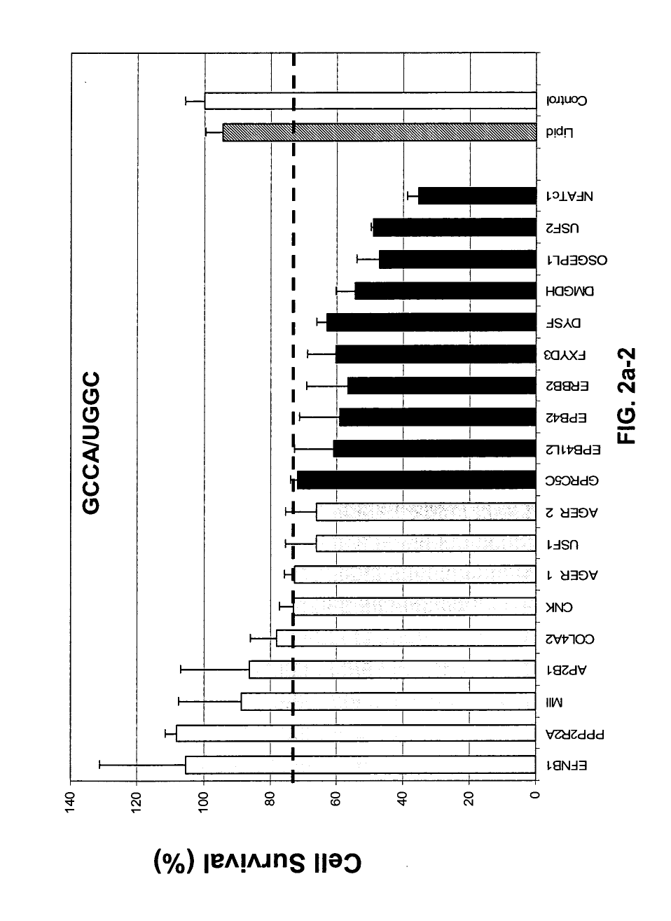

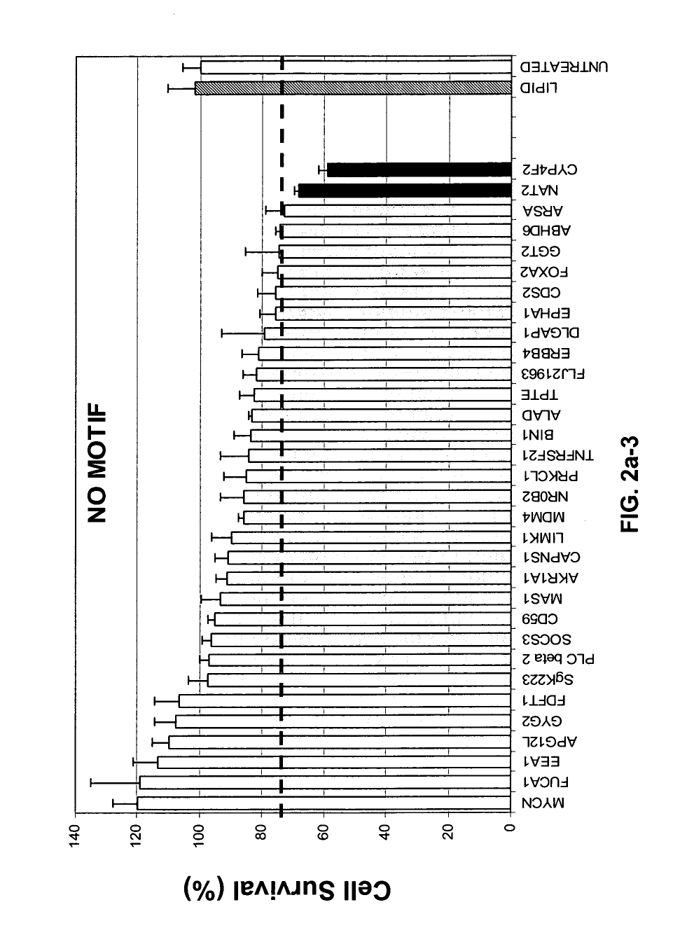

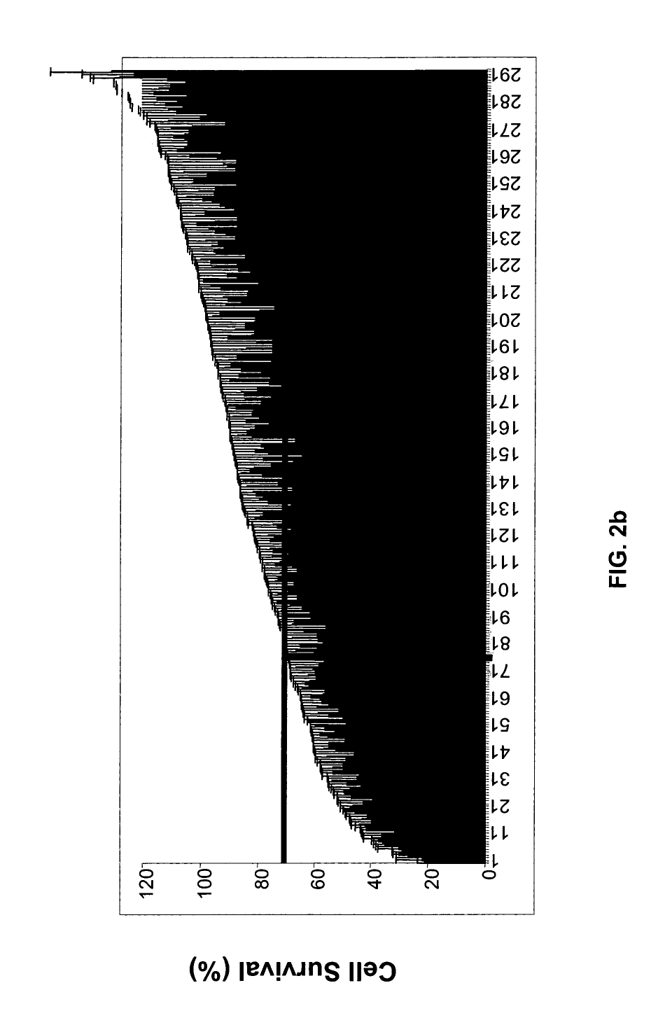

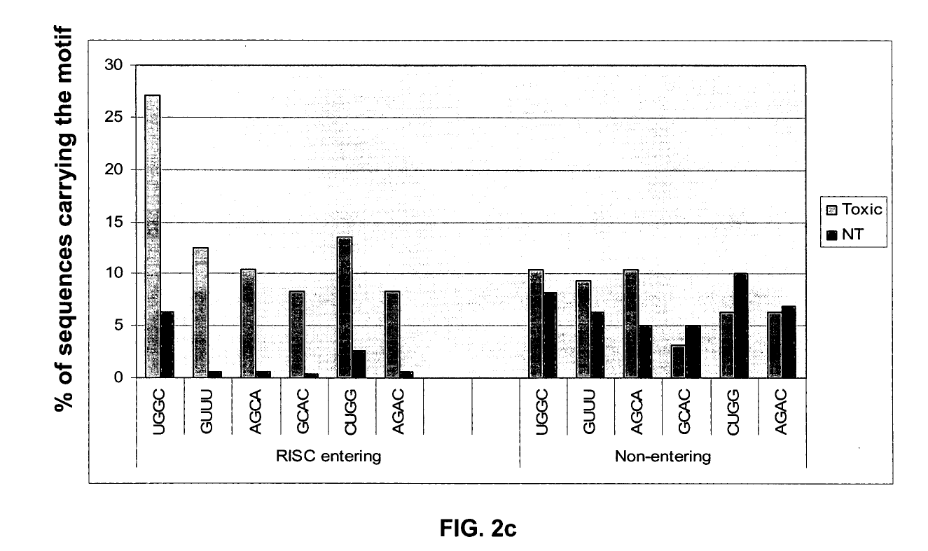

Toxic and non-toxic sequences were sorted into separate groups and analyzed to identify one or more motifs that were present in high frequencies in the toxic collection, but absent or rarely observed in the non-toxic group. The analysis of this data set identified three motifs, A/G UUU A/G/U, G/C AAA G/C and GCCA (or their complements), that exhibited the desired distribution (FIG. 2a). The A/G UUU A/G/U motif (or its complement(s)) was observed in the sense strand of 50% of the toxic siRNAs, but was found in only 11% of the sense strand of non-toxic duplexes. Similarly, the G/C AAA G/C motif (or its complement(s)) was found in the sense strand of 75% of the siRNAs in the toxic group, but only 33% of the sense strands of non-toxic duplexes. The third toxic motif (GCCA) or its complement was observed in six of the 12 toxic sequences (50%) but only once in the sense strands of non-toxic sequences (2.8%). P values were calculated to determine the relevance of the difference in the frequency of observance of each motif in toxic and non-toxic siRNA. For the A/G UUU A/G/U and G/C/AAA G/C, the P values were 0.031 and 0.0077, respectively. For the GCCA sequence, the P value was determined to be 0.000037.

siRNAs that contained either the UUU/AAA, GCCA/UGGC, or neither motif were randomly chosen and assessed using the toxicity assay. Sequences of the siRNA used in this study include the following:

TABLE IV

TOXICITY ASSAY ASSESSMENT SEQUENCES

| ACCESSION | | sIRNA SEQUENCE | SEQ. ID |
|---|---|---|---|
| No. | GENE NAME | (SENSE, 5′→3′) | NO. |
| NM_005990 | STK10 | gaaacgagauuccuucauc | 209 |
| AY406545 | MADH6 | caagaucgguuuuggcaua | 210 |
| NM_170679 | SKP1A | caaacaaucugugacuauu | 211 |
| NM_002257 | KLK1 | caacuuguuugacgacgaa | 212 |
| NM_000942 | PPIB | gaaaggauuuggcuacaaa | 213 |
| NM_005083 | U2AF1L1 | gagcauguuuacaacguuu | 214 |
| NM_000942 | PPIB | ggaaagacuguuccaaaaa | 215 |
| NM_006622 | SNK | acauuuacauucucuugga | 216 |
| NM_000942 | PPIB | gaaagagcaucuacgguga | 217 |
| NM_005379 | MYO1A | acaaggagauuuauaccua | 218 |
| NM_002620 | PF4V1 | aggaacauuuggagaguua | 219 |
| NM_005627 | Sgk1 | caucguuuauagagacuua | 220 |
| NM_022550 | XRCC4 | gaaaguaagcagaaucuau | 221 |
| AY313906 | SARS SEP | aaccaacgguuuacgucua | 222 |
| NM_181523 | PIK3R1 | gaaagacaagagaccaaua | 223 |
| NM_020183 | ARNUL2 | caacagcgauuuuaggaua | 224 |
| NM_018131 | C10ORF3 | ggaaacagcugcucauuca | 225 |
| NM_139025 | ADAMTS13 | acauuuggcugugauggua | 226 |
| NM_005767 | P2RY5 | gaaacuacaacuuacauga | 227 |
| NM_147199 | MRGX1 | gaugauguuuuccuacuuu | 228 |
| NM_001892 | CSNK1A1 | agaauuugcgauguacuua | 229 |
| NM_006930 | SKP1A | agguuugcuugauguuaca | 230 |
| M15077 | PPYLUC | cgaaaggucuuaccggaaa | 231 |
| NM_006257 | PRKCQ | caaagaguaugucgaauca | 232 |
| NM_018131 | C10ORF3 | aaggaaagcugacugauaa | 233 |
| NM_013391 | DMGDH | caucaaagcugccauggaa | 234 |
| BC025733 | FADD | cagcauuuaacgucauaug | 235 |
| NM_005541 | INPP5D | auugcguuuacacuuacag | 236 |
| NM_006395 | GSA7 | gaucaaagguuuucacuaa | 237 |
| AC146999 | Human | caaaccagcgcgcuaauga | 238 |
| | Herpes- | | |
| | virus 5 | | |
| NM_153202 | ADAM33 | caaacagcgucuccuggaa | 239 |
| NM_005508 | CCR4 | gaaagcauauacagcaauu | 240 |
| NM_002605 | PDE8A | caaagaagauaaccaaugu | 241 |
| NM_000455 | STK11 | gaaacauccuccggcugaa | 242 |
| AF493910 | PALA | gagcagauuuuaagaguaa | 243 |
| NM_012184 | FOXD4L1 | ggacaauuuugcagcaaca | 244 |
| NM_001273 | CHD4 | caaaggugcugcugaugua | 245 |
| NM_002434 | MPG | acaucauuuacggcaugua | 246 |
| NM_004429 | EFNB1 | ccacaccgcuggccaagaa | 247 |
| NM_002717 | PPP2R2A | uaucaagccugccaauaug | 248 |
| XM_110671 | M11 | ucaauaagccaucuucuaa | 249 |
| NM_001282 | AP2B1 | gagcuaaucugccacauug | 250 |
| NM_001846 | COL4A2 | cgaaggcgguggccaauca | 251 |
| AF100153 | CNK | gcacauccguuggccauca | 252 |
| NM_001136 | AGER | gccaggcaaugaacaggaa | 253 |
| NM_007122 | USF1 | ggaagccagcgcucaauug | 254 |
| NM_001136 | AGER | gcgagccacuggugcugaa | 255 |
| NM_018653 | GPRC5C | ccaccuccguugccauaug | 256 |
| NM_001431 | EPB41L2 | gaaggacucuagccaguua | 257 |
| NM_000119 | EPB42 | gaccacaccuugccaucaa | 258 |
| NM_004448 | ERBB2 | gcaguuaccagugccaaua | 259 |
| NM_005971 | FXYD3 | ggacgccaaugaccuagaa | 260 |
| NM_003494 | DYSF | gaacuaugcugccaugaag | 261 |
| NM_013391 | DMGDH | caucaaagcugccauggaa | 262 |
| NM_022353 | OSGEPL1 | agacauugcugccacagua | 263 |
| NM_003367 | USF2 | ggccaguucuacgucauga | 264 |
| NM_172390 | NFATc1 | gccaggagcugaacauuaa | 265 |
| NM_005378 | MYCN | cacguccgcucaagagugu | 266 |
| NM_000147 | FUCA1 | uaacaaugcugggaauuca | 267 |
| NM_003566 | EEA1 | agacagagcuugagaauaa | 268 |
| NM_004707 | APG12L | uguugcagcuuccuacuuc | 269 |
| NM_003918 | GYG2 | gaccaaggcuuacugaaua | 270 |
| NM_004462 | FDFT1 | cauaguuggugaagacaua | 271 |
| XM_291277 | SgK223 | gagcuccacuucaaugaga | 272 |
| NM_004573 | PLC beta | gaacagaaguuacguuguc | 273 |
| | 2 | | |
| NM_003955 | SOCS3 | caccuggacuccuaugaga | 274 |
| NM_203330 | CD59 | cuacaacuguccuaaccca | 275 |
| NM_002377 | MAS1 | cuacacaauugucacauua | 276 |
| NM_153326 | AKR1A1 | ugaggaggcugaguaauuc | 277 |
| NM_001749 | CAPNS1 | ccacagaacucaugaacau | 278 |
| NM_016735 | LIMK1 | ucaacuucaucacugagua | 279 |
| NM_002393 | MDM4 | cgucagagcuucuccguaa | 280 |
| NM_021969 | NR0B2 | cguagccgcugccuaugua | 281 |
| NM_002741 | PRKCL1 | acagcgacguguucucuga | 282 |
| NM_014452 | TNFRSF21 | cagaaggccucgaaucuca | 283 |
| NM_139343 | BIN1 | gcucaaggcuggugaugug | 284 |
| NM_001003945 | ALAD | gaugacauacagccuauca | 285 |
| NM_013315 | TPTE | uuuauucgauuccucguua | 286 |
| NM_024560 | FLJ21963 | ucgaguggaugauguaaua | 287 |
| L07868 | ERBB4 | aggaucugcauagagucuu | 288 |
| NM_001003809 | DLGAP1 | caaccuggauggugacaug | 289 |
| NM_005232 | EPHA1 | ugaagaacgguaccagaug | 290 |
| NM_003818 | CDS2 | gugagacagugacggauua | 291 |
| NM_153675 | FOXA2 | acgaacaggugaugcacua | 292 |
| XM_496495 | GGT2 | aauaaugaauggacgacuu | 293 |
| NM_020676 | ABHD6 | gaugaccuguccauagaug | 294 |
| NM_000487 | ARSA | ucuaugaccuguccaagga | 295 |
| AF348074 | NAT2 | auacagaucuggucgaguu | 296 |
| U02388 | CYP4F2 | cauauugacuuccuguauu | 297 |

Results of these studies are shown in FIG. 2b and show that inclusion of either motif in siRNA greatly enhances the frequency of toxicity. Again, this finding supports the hypothesis that siRNA carrying either motif exhibit enhance toxicity.

To further elucidate the identity and mechanism of toxic sequences, the following procedures were performed: first, 297 siRNA were collected and the RISC-entry strand bias was determined using standard thermodynamic calculation. A more detailed description of thermodynamic calculations can be found in the following patent applications: U.S. Provisional Patent Application Ser. No. 60/426,137, filed Nov. 14, 2002; U.S. Provisional Patent Application Ser. No. 60/502,050, filed Sep. 10, 2003; U.S. patent application Ser. No. 10/714,333, filed Nov. 14, 2003; International Patent Application No. PCT/US2003/036787, filed Nov. 14, 2003 and published as WO 2004/045543 A2 on Jun. 3, 2004; U.S. patent application Ser. No. 10/940,892, filed Sep. 14, 2004; and International Patent Application No. PCT/US04/14885, filed May 12, 2004; each of the foregoing applications are incorporated herein by reference. Subsequently the toxicity of each siRNA was assessed using the previously described toxicity assay. Lastly, a statistical analysis was performed whereby the frequency at which all-possible 4mers was determined. This data was then sorted based on toxicity (i.e., toxic vs. non-toxic), and the P-value was determined using Standard T-Tests to identify sequences that were present (at high frequencies) in the RISC-entering strand of toxic sequences, but not non-toxic sequences.

The identity of the RISC-entering strand (preferred strand, listed 5′ 3′) for the sequences used in this study are identified below:

TABLE V

RISC-ENTERING STRAND STUDY SEQUENCES

| | | PREFERRED | SEQ. |
|---|---|---|---|
| SEQ NAME | SEQUENCE | STRAND | ID NO. |
| GAPDH12 | aaguugucauggaugaccu | AS | 298 |
| SMART_A48 | agugaguacacaaaugucc | AS | 299 |
| SMART_A47 | uucuuggcacucgguguuc | AS | 300 |
| SMART_A46 | auacucuucaaauuuccgg | AS | 301 |
| SMART_B_20 | uacaugccguaaaugaugu | AS | 302 |
| DBI54 | agaugccaugaaagcuuac | S | 303 |
| SMART_B_40 | uacaucagcagcaccuuug | AS | 304 |
| SMART_B_59 | uuaauguucagcuccuggc | AS | 305 |
| DBI45 | uuccaaggaagaugccaug | S | 306 |
| SMART_B_18 | uuauugacaaacuccugga | AS | 307 |
| SMART_B_19 | uguugcugcaaaauugucc | AS | 308 |
| SMART_A45 | agagacuguagguaguuuc | AS | 309 |
| SMART_B_39 | uucagccggaggauguuuc | AS | 310 |
| SMART_B_38 | acauugguuaucuucuuug | AS | 311 |
| GAPDH6 | augaccuuggccaggggug | AS | 312 |
| SMART_B_17 | uuacucuuaaaaucugcuc | AS | 313 |
| SMART_A43 | uuauugacaaacuccugga | AS | 314 |
| LUC6 | aaaaucagagagauccuca | S | 315 |
| SMART_B_37 | aauugcuguauaugcuuuc | AS | 316 |
| SMART_B_58 | ucaugacguagaacuggcc | AS | 317 |
| SMART_B_36 | uuccaggagacgcuguuug | AS | 318 |
| GAPDH8 | auggaugaccuuggccagg | AS | 319 |
| SMART_A44 | caauggcucaaguaucugc | AS | 320 |
| LUC30 | uguuuguggacgaaguacc | S | 321 |
| SMART_C_61 | uucgagggagaaguucuuc | AS | 322 |
| SMART_B_35 | uuccaggagacgcuguuug | AS | 323 |
| SMART_B_16 | uuagugaaaaccuuugauc | AS | 324 |
| SMART_B_57 | uacuguggcagcaaugucu | AS | 325 |
| LUC79 | ugcuccaaaacaacaacgg | AS | 326 |
| SMART_C_60 | caugugagcagguccuccg | AS | 327 |
| DBI29 | augagcugaaagggacuuc | S | 328 |
| SMART_C_58 | uauugguugucacugauca | AS | 329 |
| LUC14 | uucuugcgucgaguuuucc | AS | 330 |
| SMART_C_57 | uuccaagaccugccuacca | S | 331 |
| LUC38 | uugcgcggaggaguugugu | S | 332 |
| GAPDH10 | ugucauggaugaccuuggc | AS | 333 |
| GAPDH2 | cugcuuagcaccccuggcc | S | 334 |
| DBI27 | agucccuuucagcucauuc | AS | 335 |
| SMART_A42 | auguuuacuaccaaaugga | S | 336 |
| LUC74 | uuuggagcacggaaagacg | S | 337 |
| LUC53 | uuguuacuugacuggcgac | AS | 338 |
| LUC85 | aacuucccgccgccguugu | S | 339 |
| DBI44 | auggcaucuuccuuggaag | AS | 340 |
| SMART_B_56 | uuccauggcagcuuugaug | AS | 341 |
| SMART_B_33 | uuccauggcagcuuugaug | AS | 342 |
| LUC75 | ucuuuccgugcuccaaaac | AS | 343 |
| SMART_A40 | aguugcuucaaaucugcuc | AS | 344 |
| GAPDH5 | agcaccccuggccaagguc | S | 345 |
| SMART_B_15 | auugcguuuacacuuacag | S | 346 |
| SMART_D_32 | auguucaugaguucugugg | AS | 347 |
| SMART_B_32 | ugauucgacauacucuuug | AS | 348 |
| SMART_B_31 | ucauuagcgcgcugguuug | AS | 349 |
| DBI47 | uucauggcaucuuccuugg | AS | 350 |
| LUC18 | ucgaguuuuccgguaagac | AS | 351 |
| LUC72 | ucaucgucuuuccgugcuc | AS | 352 |
| LUC11 | ugauuuuucuugcgucgag | AS | 353 |
| LUC19 | aggucuuaccggaaaacuc | S | 354 |
| DBI49 | aaggaagaugccaugaaag | S | 355 |
| SMART_C_55 | auaggcagcagcgaguucc | AS | 356 |
| SMART_A38 | aacaguggcauaggcuuuc | AS | 357 |
| LUC73 | aucgucuuuccgugcucca | AS | 358 |
| LUC3 | agagauccucauaaaggcc | S | 359 |
| LUC9 | ucucucugauuuuucuugc | AS | 360 |
| LUC31 | uacuucguccacaaacaca | AS | 361 |
| DBI30 | ugagcugaaagggacuucc | S | 362 |
| LUC2 | uuggccuuuaugaggaucu | AS | 363 |
| LUC60 | agagaucguggauuacguc | S | 364 |
| LUC86 | aacggcggcgggaaguuca | AS | 365 |
| GAPDH1 | aacugcuuagcaccccugg | S | 366 |
| SMART_C_54 | uauccuucaugcccauucc | AS | 367 |
| DBI51 | agcuuucauggcaucuucc | AS | 368 |
| SMART_B_54 | uucuaggucauuggcgucc | AS | 369 |
| SMART_B_11 | aaaguaggaaaacaucauc | AS | 370 |
| GAPDH4 | uuagcaccccuggccaagg | S | 371 |
| SMART_B_53 | uauuggcacugguaacugc | AS | 372 |
| DBI50 | aggaagaugccaugaaagc | S | 373 |
| SMART_B_30 | uuuccgguaagaccuuucg | AS | 374 |
| LUC21 | uuuccgguaagaccuuucg | AS | 375 |
| SMART_B_29 | ucauguaaguuguaguuuc | AS | 376 |
| LUC27 | uggacgaaguaccgaaagg | S | 377 |
| SMART_D_31 | uuauucucaagcucugucu | AS | 378 |
| SMART_A39 | caaguggccagggcauagc | AS | 379 |
| SMART_C_53 | augaggaugauggguauga | S | 380 |
| SMART_C_52 | uaguugaccagcucauccg | AS | 381 |
| SMART_B_52 | uugauggcaaggugugguc | AS | 382 |
| SMART_B_12 | uaaguacaucgcaaauucu | AS | 383 |
| SMART_B_28 | ugaaugagcagcuguuucc | AS | 384 |
| LUC17 | cuuaccggaaaacucgacg | S | 385 |
| SMART_B_10 | uaccaucacagccaaaugu | AS | 386 |
| LUC64 | acggaaaaagagaucgugg | S | 387 |
| SMART_B_51 | uaacuggcuagaguccuuc | AS | 388 |
| LUC58 | acuggcgacguaauccacg | AS | 389 |
| LUC7 | aggaucucucugauuuuuc | AS | 390 |
| SMART_B_50 | cauauggcaacggaggugg | AS | 391 |
| LUC59 | agaucguggauuacgucgc | S | 392 |
| LUC24 | aaguaccgaaaggucuuac | S | 393 |
| LUC33 | ucguccacaaacacaacuc | AS | 394 |
| SMART_B_49 | uucagcaccaguggcucgc | AS | 395 |
| LUC4 | agagagauccucauaaagg | S | 396 |
| SMART_B_48 | caauugagcgcuggcuucc | AS | 397 |
| SMART_A36 | aaucacuccaacugaaucc | AS | 398 |
| SMART_C_49 | aagacaauagucccuugga | S | 399 |
| SMART_A35 | agaaccuccauccaugugc | AS | 400 |
| GAPDH3 | uuggccaggggugcuaagc | AS | 401 |
| LUC15 | cuugcgucgaguuuuccgg | AS | 402 |
| SMART_A34 | aauggcuuugaaucuuugg | S | 403 |
| SMART_D_30 | uacauaggcagcggcuacg | AS | 404 |
| DBI89 | agcuaaagaaaaaauacgg | S | 405 |
| DBI52 | aagcuuucauggcaucuuc | AS | 406 |
| SMART_B_9 | uauccuaaaaucgcuguug | AS | 407 |
| SMART_C_48 | aaauucaucaucgaaguac | AS | 408 |
| SMART_A32 | uaaagguuaacauccgguc | AS | 409 |
| SMART_B_27 | uauuggucucuugucuuuc | AS | 410 |
| SMART_C_43 | aaugugcccguccuugucc | AS | 411 |
| LUC69 | uuuccgucaucgucuuucc | AS | 412 |
| LUC78 | guuguuguuuuggagcacg | S | 413 |
| SMART_D_28 | cacaucaccagccuugagc | AS | 414 |
| LUC20 | aaaggucuuaccggaaaac | S | 415 |
| SMART_D_27 | ugaggaggcugaguaauuc | S | 416 |
| LUC61 | aaagagaucguggauuacg | S | 417 |
| SMART_A30 | uuaauguccacugcgucga | AS | 418 |
| SMART_C_47 | ucguacacuagcacauugc | AS | 419 |
| SMART_C_45 | uuggacccguacuucauca | S | 420 |
| SMART_B_47 | uuccuguucauugccuggc | AS | 421 |
| SMART_B_46 | ugauggccaacggaugugc | AS | 422 |
| LUC34 | aggaguuguguuuguggac | S | 423 |
| SMART_C_41 | auagugaaggcagcuguga | AS | 424 |
| SMART_C_50 | guaucggaggcgugugucc | AS | 425 |
| SMART_C_46 | caucugaggaggcaccugc | AS | 426 |
| LUC49 | uuucgcgguuguuacuuga | AS | 427 |
| SMART_A29 | aauuucuggagcccuguac | AS | 428 |
| SMART_B_26 | auagauucugcuuacuuuc | AS | 429 |
| LUC25 | aagaccuuucgguacuucg | AS | 430 |
| SMART_C_44 | cauaguaguagcccagugc | AS | 431 |
| SMART_A28 | uaguaaagcgccuccggcc | AS | 432 |
| SMART_A27 | uagucaaugcuuucuucag | AS | 433 |
| SMART_D_26 | uaauccgucacugucucac | AS | 434 |
| LUC41 | aaaaaguugcgcggaggag | S | 435 |
| LUC37 | aaacacaacuccuccgcgc | AS | 436 |
| LUC54 | acgucgccagucaaguaac | S | 437 |
| SMART_D_25 | uggguuaggacaguuguag | AS | 438 |
| SMART_C_42 | uauuguaacacgccuaaca | AS | 439 |
| SMART_A26 | uacaaaggcaguaaggcuc | AS | 440 |
| LUC82 | aaaacaacaacggcggcgg | AS | 441 |
| SMART_A25 | ucucucggcagucuugucg | AS | 442 |
| SMART_C_28 | auaaccggaaguccuccuc | AS | 443 |
| LUC43 | uccgcgcaacuuuuucgcg | AS | 444 |
| SMART_D_24 | ugauaggcuguaugucauc | AS | 445 |
| SMART_A24 | aaugauacgaucgaguucc | AS | 446 |
| SMART_C_36 | acagucccaucuucaucac | AS | 447 |
| DBI65 | aagcuuacaucaacaaagu | S | 448 |
| SMART_B_45 | ugauuggccaccgccuucg | AS | 449 |
| SMART_B_7 | uaagucucuauaaacgaug | AS | 450 |
| LUC16 | uaccggaaaacucgacgca | S | 451 |
| SMART_C_35 | uaaugugcccguccuuguc | AS | 452 |
| SMART_B_6 | uaacucuccaaauguuccu | AS | 453 |
| LUC28 | uuucgguacuucguccaca | AS | 454 |
| SMART_C_38 | aagaagcgaugcugcauga | AS | 455 |
| LUC44 | accgcgaaaaaguugcgcg | S | 456 |
| DBI48 | uuucauggcaucuuccuug | AS | 457 |
| SMART_D_23 | uuacggagaagcucugacg | AS | 458 |
| LUC46 | aacaaccgcgaaaaaguug | S | 459 |
| SMART_A23 | uuccaugcaaaugcugauc | AS | 460 |
| SMART_D_22 | uacucagugaugaaguuga | AS | 461 |
| DBI75 | uagcucuucuacuuuguug | AS | 462 |
| LUC29 | uuuguggacgaaguaccga | S | 463 |
| DBI88 | gagcuaaagaaaaaauacg | S | 464 |
| SMART_D_21 | uauuacaucauccacucga | AS | 465 |
| SMART_C_19 | auaguugaccagcucaucc | AS | 466 |
| SMART_D_20 | aagacucuaugcagauccu | AS | 467 |
| DBI62 | uuguugauguaagcuuuca | AS | 468 |
| DBI37 | aaagggacuuccaaggaag | S | 469 |
| LUC47 | acuuuuucgcgguuguuac | AS | 470 |
| SMART_D_19 | ucucauugaaguggagcuc | AS | 471 |
| SMART_D_18 | uauucaguaagccuugguc | AS | 472 |
| SMART_A22 | aacuuguacaggucagucu | AS | 473 |
| LUC8 | aagaaaaaucagagagauc | S | 474 |
| SMART_D_17 | acacucuugagcggacgug | AS | 475 |
| SMART_C_37 | uucuuguuaacuacugcca | AS | 476 |
| LUC80 | cuccaaaacaacaacggcg | AS | 477 |
| SMART_C_34 | ugaacaugaaccgcccucc | AS | 478 |
| DBI81 | uuucuuuagcucuucuacu | AS | 479 |
| LUC63 | auccacgaucucuuuuucc | AS | 480 |
| LUC1 | auccucauaaaggccaaga | S | 481 |
| SMART_B_5 | uagguauaaaucuccuugu | AS | 482 |
| SMART_C_33 | acucucgggagccuuguuc | AS | 483 |
| LUC36 | acaaacacaacuccuccgc | AS | 484 |
| DBI87 | agagcuaaagaaaaaauac | S | 485 |
| SMART_C_20 | uuacacaacacuucgaugg | AS | 486 |
| SMART_C_31 | aauuccaaggugcguguug | AS | 487 |
| SMART_A21 | uacugcaacugcguguagc | AS | 488 |
| SMART_C_29 | aucaucgaaguaccuugug | AS | 489 |
| DBI55 | uguaagcuuucauggcauc | AS | 490 |
| LUC23 | guaccgaaaggucuuaccg | S | 491 |
| DBI77 | uuuagcucuucuacuuugu | AS | 492 |
| SMART_B_25 | ucaccguagaugcucuuuc | AS | 493 |
| SMART_A20 | acuugauccagagaaccuc | AS | 494 |
| SMART_D_15 | uaaugugacaauuguguag | AS | 495 |
| DBI63 | uuuguugauguaagcuuuc | AS | 496 |
| 4SMART_A19 | uuugguguaaccucccuug | AS | 497 |
| SMART_A18 | caguaaaucagauaguagc | AS | 498 |
| DBI67 | cuacuuuguugauguaagc | AS | 499 |
| SMART_A17 | uacugcaacaucuccgugc | AS | 500 |
| SMART_A16 | ucaauagugcaaucauuuc | AS | 501 |
| LUC83 | aacaacaacggcggcggga | AS | 502 |
| SMART_A15 | uacuucaucacuaucuccc | AS | 503 |
| DBI1 | acgggcaaggccaaguggg | S | 504 |
| SMART_C_32 | ucuaguagcagcgucuccc | AS | 505 |
| SMART_C_21 | aaguccuccagguaguugg | AS | 506 |
| SMART_C_9 | uucucagggaccucaauag | AS | 507 |
| DBI57 | ugccaugaaagcuuacauc | S | 508 |
| SMART_B_43 | uuagaagauggcuuauuga | AS | 509 |
| DBI6 | caaggccaagugggaugcc | S | 510 |
| SMART_C_26 | guauccguagugcuugucc | AS | 511 |
| SMART_C_27 | ugaaccauauucugucuuc | AS | 512 |
| DBI79 | aaaguagaagagcuaaaga | S | 513 |
| SMART_A14 | auguacuucaucacuaucu | AS | 514 |
| SMART_B_24 | uuuuuggaacagucuuucc | AS | 515 |
| SMART_C_18 | uuacacaacacuucgaugg | AS | 516 |
| DBI73 | aucaacaaaguagaagagc | S | 517 |
| SMART_A13 | uaucucccgguugucguac | AS | 518 |
| DBI3 | aucccacuuggccuugccc | AS | 519 |
| SMART_B_3 | aaacguuguaaacaugcuc | AS | 520 |
| SMART_B_23 | uuuguagccaaauccuuuc | AS | 521 |
| DBI61 | augaaagcuuacaucaaca | S | 522 |
| SMART_C_4 | uugauaucaccacguaccu | AS | 523 |
| SMART_C_14 | uucuugaggaggaaguagc | AS | 524 |
| DBI58 | ugauguaagcuuucauggc | AS | 525 |
| DBI33 | cuuggaagucccuuucagc | AS | 526 |
| SMART_A12 | aauaaguccagagcuuugg | AS | 527 |
| DBI22 | cuuucagcucauuccaggc | AS | 528 |
| DBI4 | caucccacuuggccuugcc | AS | 529 |
| SMART_B_2 | uucgucgucaaacaaguug | AS | 530 |
| SMART_A11 | uucuacuucaauccucuug | AS | 531 |
| DBI66 | uacuuuguugauguaagcu | AS | 532 |
| SMART_B_22 | aauagucacagauuguuug | AS | 533 |
| DBI19 | ucagcucauuccaggcauc | AS | 534 |
| SMART_A10 | ucaagguggaucagcuucc | AS | 535 |
| DBI17 | agcucauuccaggcauccc | AS | 536 |
| SMART_A9 | uucgaagucaucgucuuug | AS | 537 |
| SMART_C_11 | uuggaaugaacacccuugc | AS | 538 |
| DBI38 | aagggacuuccaaggaaga | S | 539 |
| SMART_D_12 | uguugcagcuuccuacuuc | S | 540 |
| DBI21 | uuucagcucauuccaggca | AS | 541 |
| SMART_B_1 | uaugccaaaaccgaucuug | AS | 542 |
| DBI26 | gucccuuucagcucauucc | AS | 543 |
| SMART_C_17 | uucgauuccacagugaucc | AS | 544 |
| DBI31 | uggaagucccuuucagcuc | AS | 545 |
| SMART_D_11 | uaugucuucaccaacuaug | AS | 546 |
| DBI70 | uacaucaacaaaguagaag | S | 547 |
| SMART_A8 | ucaagugucuccuaauaug | S | 548 |
| DBI32 | uuggaagucccuuucagcu | AS | 549 |
| DBI85 | auuuuuucuuuagcucuuc | AS | 550 |
| SMART_A7 | uauuguuggauuucauguc | AS | 551 |
| DBI39 | aucuuccuuggaagucccu | AS | 552 |
| DBI40 | caucuuccuuggaaguccc | AS | 553 |
| DBI82 | uuuucuuuagcucuucuac | AS | 554 |
| SMART_A6 | auacgaucgaguuccuuga | AS | 555 |
| DBI64 | aaagcuuacaucaacaaag | S | 556 |
| DBI7 | aaggccaagugggaugccu | S | 557 |
| LUC45 | caaccgcgaaaaaguugcg | S | 558 |
| DBI2 | ucccacuuggccuugcccg | AS | 559 |
| SMART_C_10 | aaucauugcaggucagauc | AS | 560 |
| SMART_A5 | ucaaaucugcucucucugc | AS | 561 |
| DBI84 | uuuuuucuuuagcucuucu | AS | 562 |
| SMART_A4 | uauggaugauguucuuugc | AS | 563 |
| DBI8 | aggccaagugggaugccug | S | 564 |
| DBI13 | aagugggaugccuggaaug | S | 565 |
| DBI59 | uugauguaagcuuucaugg | AS | 566 |
| SMART_B_41 | uucuuggccagcggugugg | AS | 567 |
| SMART_C_6 | aguacaacugcaacaagug | S | 568 |
| DBI16 | ugggaugccuggaaugagc | S | 569 |
| DBI42 | gacuuccaaggaagaugcc | S | 570 |
| DBI24 | cuggaaugagcugaaaggg | S | 571 |
| SMART_D_9 | ugaagaacgguaccagaug | S | 572 |
| SMART_B_42 | uaucaagccugccaauaug | S | 573 |
| SMART_A2 | aaacagaucuuuacaagcu | S | 574 |
| SMART_C_8 | aucaguggacacuaugaca | S | 575 |
| DBI34 | cugaaagggacuuccaagg | S | 576 |
| SMART_C_7 | uuacagugcgacagcuuga | S | 577 |
| SMART_C_5 | guugugaaugacguauugg | AS | 578 |
| DBI68 | ucuacuuuguugauguaag | AS | 579 |
| SMART_C_1 | acguuguagaaguugugcc | AS | 580 |
| DBI10 | uccaggcaucccacuuggc | AS | 581 |
| DBI18 | cagcucauuccaggcaucc | AS | 582 |
| SMART_D_8 | ugagauucgaggccuucug | AS | 583 |
| SMART_D_7 | aauacaggaagucaauaug | AS | 584 |
| SMART_D_5 | uaacaaugcugggaauuca | S | 585 |
| DBI11 | uuccaggcaucccacuugg | AS | 586 |
| DBI36 | uuccuuggaagucccuuuc | AS | 587 |
| SMART_D_4 | ucucauaggaguccaggug | AS | 588 |
| DBI12 | auuccaggcaucccacuug | AS | 589 |
| SMART_D_3 | uagugcaucaccuguucgu | AS | 590 |

The results of these studies are as follows: FIG. 2c shows the distribution of toxic and non-toxic sequences within the population. When the described statistical analysis was performed on the sequences, six 4-mer motifs that are over-represented in the RISC-entering strand of toxic siRNA were identified. The table below reports the frequency at which these sequences are associated with the RISC-entering strand of toxic sequences, the RISC-entering strand of non-toxic sequences, and the P-values that describe the relevance of the differences at which these sequences are observed in the strands of both toxic and non-toxic groups. A graphical representation of this data is presented in FIG. 2d and shows that (1) while each of the sequences is strongly represented in the RISC-entering strand of toxic sequences, they are found at a low frequency in RISC-entering strand of non-toxic sequences, and (2) the frequency at which these sequences are found in the non-entering strand of toxic and non-toxic siRNA is roughly equivalent. Together, these properties strongly suggest that these sequences are associated with the observed siRNA-induced toxicity.

TABLE VI

| | FREQUENCY ANALYSIS | | |
|---|---|---|---|
| | % OF TOXIC | % OF NOT TOXIC | P VALUE |
| UGGC | 27.08333 | 6.289308 | 1.31E−06 |
| GUUU | 12.5 | 0.628931 | 1.17E−05 |
| AGCA | 10.41667 | 0.628931 | 8.46E−05 |
| GCAC | 8.333333 | 0 | 9.53E−05 |
| CUGG | 13.54167 | 2.515723 | 0.000288 |
| AGAC | 8.333333 | 0.628931 | 0.000581 |

### Example 2

Dose Dependence Toxicity

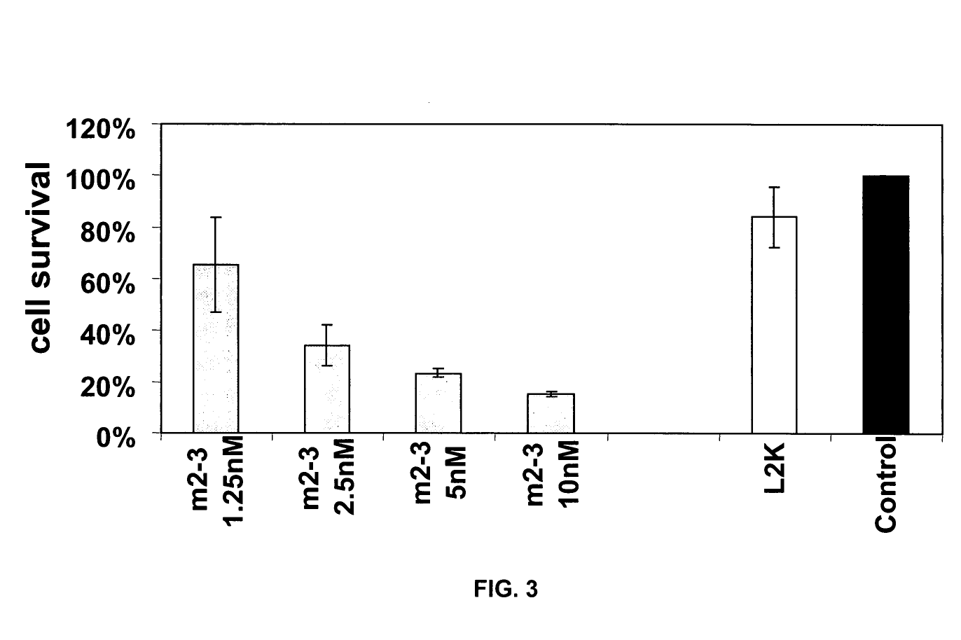

To determine whether the observed toxicity induced by Mek2-3 (MAP2K2-3, m23) represented a titratable event, the toxic Mek2-3 sequence (5′ UCCAGGAGUUUGUCAAUAA, sense strand (SEQ. ID NO.638)) s transfected (Lipofectamine 2000) into HeLa cells at concentrations that varied between 1.25-10 nanomolar. The total concentration of siRNA in all of the experiments was 10 nanomolar with a non-toxic mek2-1 siRNA (5′ CAAAGACGAUGACUUCGAA, sense strand (SEQ. ID NO.639)) ing up the difference at lower Mek2-3 concentrations. The results of these experiments are shown in FIG. 3 and demonstrate that the level of toxicity was proportional to the concentration of the Mek2-3 siRNA. High concentrations of Mek2-3 (10 nanomolar) induced greater than 80% cell death, while lower concentrations (e.g., 1.25 nanomolar) induced approximately 40% cell death. These results suggest that one or more essential cellular targets are being titrated.

### Example 3

### Testing the Correlation Between Toxicity and Silencing

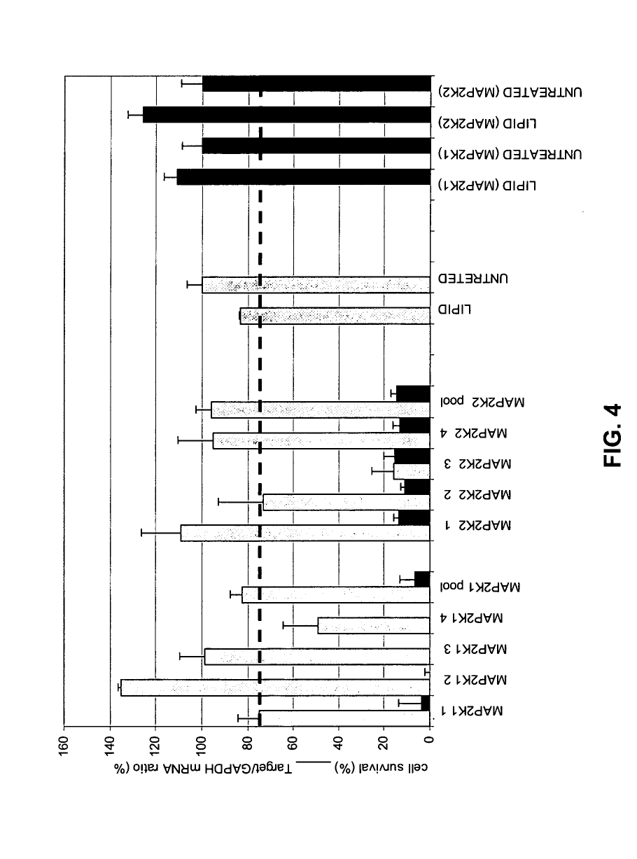

To further determine whether a correlation existed between the level of silencing induced by a given siRNA and the amount of toxicity brought on by that same sequence, the four siRNAs directed against either Mek1 (MAP2K1) or Mek2 (MAP2K2) were simultaneously tested for toxicity and gene knockdown (sequences for all of the duplexes are listed in Table 1). In these experiments, the method of siRNA transfection and the means of determining toxicity were performed as previously described. To determine the level of gene knockdown, cells transfected with each siRNA were harvested and Mek1 or Mek2 expression levels were compared with those of a control gene (GAPDH) using Quantigene® Kits (Genospectra, Fremont, Calif.) that make use of art-recognized branched DNA technology. The results of these experiments are shown in FIG. 4 and illustrate that while all eight siRNAs silence their respective target message by greater than 80%, the level of toxicity induced by each varies greatly. These data would suggest that there is no correlation between the silencing potential of the duplex and the relative toxicity of the siRNA.

### Example 4

### The Dependence of Toxicity on the RNAi Pathway

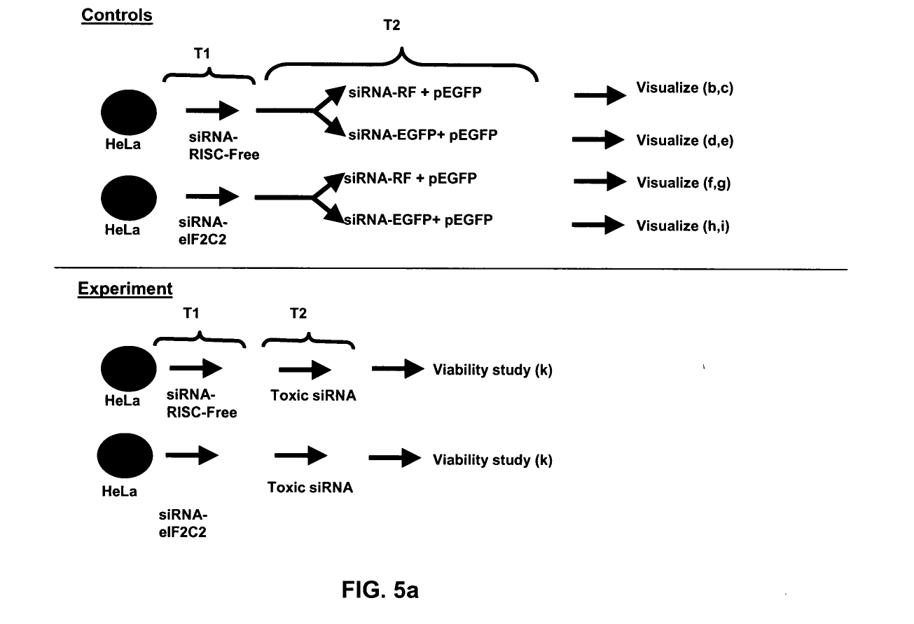

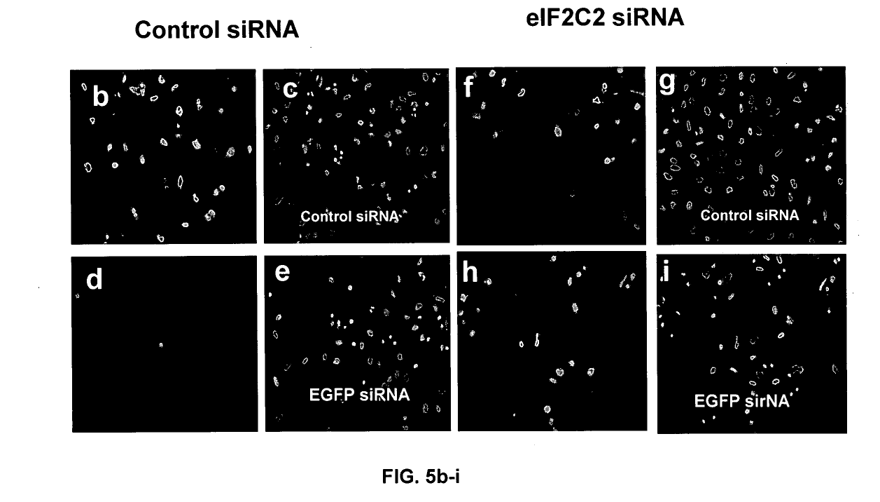

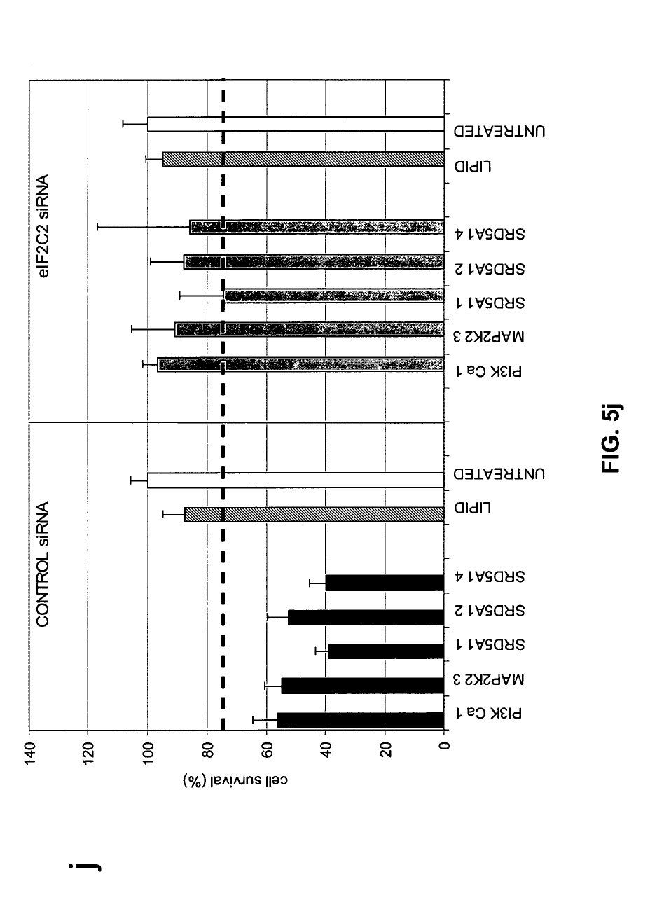

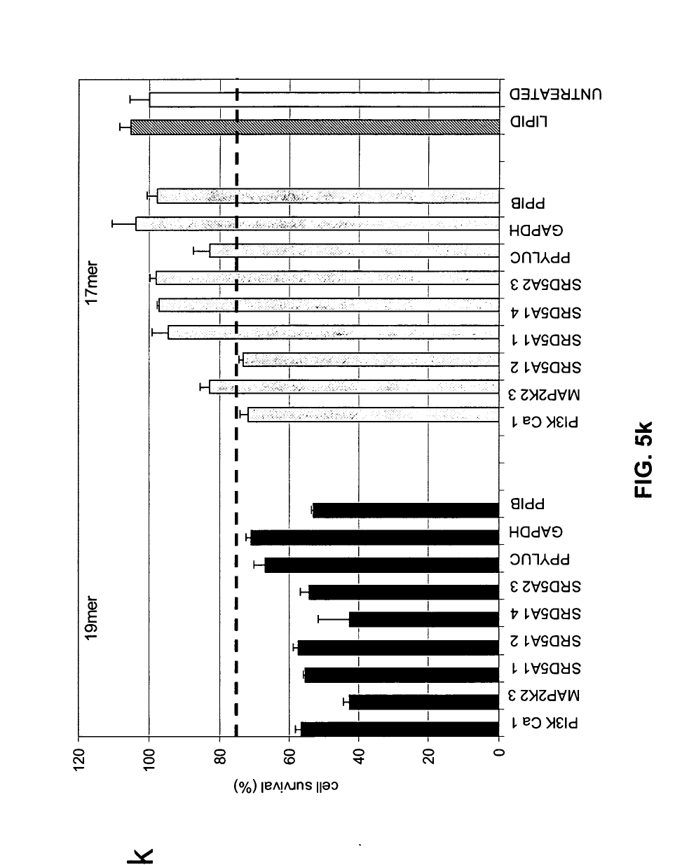

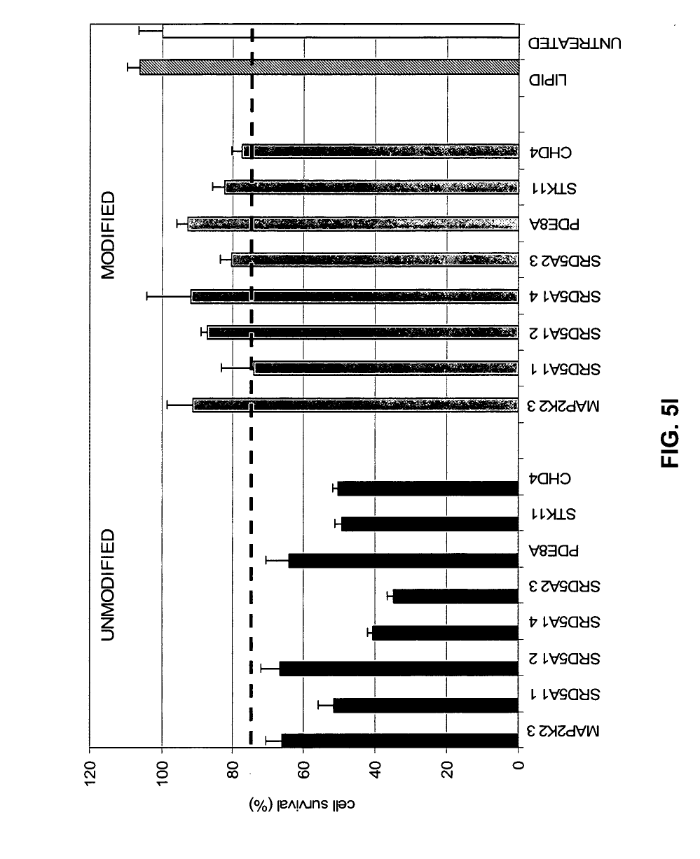

Three different approaches were used to evaluate the contributions of the RNAi pathway (and specifically siRNA off-targeting activity) to the observed siRNA-induced toxicity. First, the ability of toxic motif containing siRNA to induce cell death was investigated under circumstances where the RNAi mechanism was severely compromised. Previous studies have shown that eIF2C2(hAgo2) is responsible for RNAi-mediated mRNA cleavage and that knockdown of this gene product severely cripples the pathway (Meister, G. et al. (2004) Human Argonaute2 mediates RNA cleavage targeted by miRNAs and siRNAs. Mol Cell 15, 185-97; Tabara, H. et al. (1999) The rde-1 gene, RNA interference, and transposon silencing in C. elegans, Cell 99, 123-32. To confirm this, cells transfected with an eIF2C2-siRNA pool that induced more than 70% silencing or control siRNA were subsequently co-transfected with an EGFP expression plasmid plus either a) a control siRNA or b) an siRNA directed against EGFP (second transfection, see FIG. 5a for description of experiments). Sequences used in these experiments include the following:

TABLE VI

SEQUENCES FOR RNAI COMPROMISE STUDY

| | | SIRNA SEQUENCE | SEQ. ID |
|---|---|---|---|
| ACCESSION No. | GENE NAME | (SENSE, 5′→3′) | NO. |
| NM_012154 | eIF2C2 | gcacggaaguccaucugaa | 591 |
| | | gcaggacaaagauguauua | 592 |
| | | gggucuguggugauaaaua | 593 |
| | | guaugagaacccaauguca | 594 |
| | EGFP | gcaaagaccccaacgagaa | 595 |

As expected, eIF2C2 knockdown disabled subsequent siRNA induced silencing of EGFP (FIG. 5b-i).

When eIF2C2 minus cells (cells treated with eIF2C2 targeting siRNAs) were subsequently transfected with toxic siRNA, no effect on cell viability was observed (FIG. 5j). Sequences used in this study included the following:

TABLE VII

EIF2C2 MINUS STUDY SEQUENCES

| | | | | SEQ. |
|---|---|---|---|---|
| ACCESSION | GENE | SIRNA | SIRNA SEQUENCE | ID |
| No. | NAME | NAME | (SENSE, 5′→3′) | NO. |
| NM_006218 | PI3K Ca | PI3K Ca- | auguuuacuaccaaaugga | 596 |
| | | 1 | | |
| NM_001047 | SRD5A1 | SRD5A1 | uaacugcagccaacuauuu | 597 |
| | | 2 | | |
| NM_030662 | MAP2K2 | MAP2K2 | | |
| | | 3 | uccaggaguuugucaauaa | 598 |
| NM_001047 | SRD5A1 | SRD5A1- | gcagauacuugagccauug | 599 |
| | | 1 | | |
| NM_001047 | SRD5A1 | SRD5A1 | ccggaaauuugaagaguau | 600 |
| | | 4 | | |

As parallel experiments where cells were pre-transfected with control siRNA demonstrated levels of toxicity characteristic of these sequences, it was concluded that an uncompromised RNAi pathway was necessary for development of siRNA-induced toxicity.

In a second approach, the ability of toxic siRNA to induce cell death was tested when the size of the duplex was reduced from 19 bp to 17 bp. Previous studies have shown that duplexes that are shorter than 19 bp targeted mRNA sequences inefficiently, suggesting that Dicer and/or RISC fail to mediate RNAi when duplex sequence length drops below 19 bp (Elbashir, S. M., Martinez, J., Patkaniowska, A., Lendeckel, W. & Tuschl, T. (2001) Functional anatomy of siRNAs for mediating efficient RNAi in Drosophila melanogaster embryo lysate. EMBO Journal 20, 6877-88). Known toxic siRNA were then tested for toxicity in both the 19 bp and 17 bp format. Sequences used in this study are listed below.

TABLE VIII

17 AND 19-MER SEQUENCES TESTED FOR TOXICITY

| | 19-MER SEQUENCE | SEQ. ID | 17-MER SEQUENCE | SEQ. ID |
|---|---|---|---|---|
| GENE | (SENSE, 5′→3′) | NO. | (SENSE, 5′→3′) | NO. |
| PI3K Ca | auguuuacuaccaaaugga | 650 | auguuuacuaccaaaug | 601 |
| MAP2K2-3 | uccaggaguuugucaauaa | 651 | uccaggaguuugucaau | 602 |
| SRD5A1-1 | gcagauacuugagccauug | 652 | gcagauacuugagccau | 603 |
| SRD5A1-2 | uaacugcagccaacuauuu | 653 | uaacugcagccaacuau | 604 |
| SRD5A1-4 | ccggaaauuugaagaguau | 654 | ccggaaauuugaagagu | 605 |
| SRD5A2-3 | ggacauuuguguacucacu | 655 | ggacauuuguguacuca | 606 |
| PPYLUC | uguuuguggacgaaguacc | 656 | uuuguggacgaaguacc | 607 |
| GAPDH | ccuggccaaggucauccau | 657 | uggccaaggucauccau | 608 |
| PPIB | gagaaaggauuuggcuaca | 658 | gaaaggauuuggcuaca | 609 |

TABLE IX

TOXICITY TESTING SEQUENCES

| | | | | SEQ. |
|---|---|---|---|---|
| ACCESSION | GENE | SIRNA | SIRNA SEQUENCE | ID |
| No. | NAME | NAME | (SENSE, 5′→3′) | NO. |
| NM_006218 | P13K Ca | P13K Ca | auguuuacuaccaaaugga | 610 |
| | | 1 | | |
| NM_001047 | SRD5A1 | SRD5A1 | uaacugcagccaacuauuu | 611 |
| | | 2 | | |
| NM_030662 | MAP2K2 | MAP2K2 | uccaggaguuugucaauaa | 612 |
| | | 3 | | |
| NM_001047 | SRD5A1 | SRD5A1 | gcagauacuugagccauug | 613 |
| | | 1 | | |
| NM_001047 | SRD5A1 | SRD5A1 | ccggaaauuugaagaguau | 614 |
| | | 3 | | |
| NM_000348 | SRD5A2 | SRD5A2 | ggacauuuguguacucacu | 615 |
| | | 3 | | |
| M15077 | PPYLUC | | uguuuguggacgaaguacc | 616 |
| BC020308 | GAPDH | | ccuggccaaggucauccau | 617 |
| NM_000942 | PPIB | | gagaaaggauuuggcuaca | 618 |

When the length of known 19 bp toxic siRNA was reduced by 2 bp (17 bp total length, no disruption of the motif) the level of toxicity was reduced dramatically (FIG. 5k), suggesting again that entry and/or processing by RISC was necessary for induction of toxicity.

Finally, in a third approach, chemical modifications that eliminate RNAi-mediated off-target effects were tested for the ability to abolish siRNA-induced toxicity. Recent studies have shown that minimal chemical modification of both strands of siRNA dramatically limits off-target effects without altering target specific knockdown (see U.S. Provisional Patent Application Ser. No. 60/630,228, filed Nov. 22, 2004, entitled “Modified siRNAs with Enhanced Selectivity”; and U.S. patent application Ser. No. 11/019,831, filed Dec. 22, 2004, entitled “Modified Polynucleotides for Reducing Off-Target Effects in RNA Interference”; each of which is herein incorporated by reference). To test the effect of this modification pattern on siRNA induced toxicity, we applied the chemical modification pattern [sense strand: 2′-O-methyl modification on nucleotides 1 and 2 (counting from the 5′ end of the sense strand); antisense strand: 2′-O-methyl modification of positions 1 and 2 (counting from the 5′ end of the antisense strand), 5′ phosphorylation of the first nucleotide of the antisense strand (counting from the 5′ end of the antisense strand)] to known toxic siRNA and transfected these duplexes into HeLa cells. The sequences of the duplexes used in this study are listed in the table below:

TABLE X

SEQUENCES USED IN INVESTIGATING MODIFICATION
EFFECTS ON TOXICITY

| | | | | SEQ. |
|---|---|---|---|---|
| ACCESSION | GENE | SIRNA | SIRNA SEQUENCE | ID |
| No. | NAME | NAME | (SENSE, 5′→3′) | NO. |
| NM_006218 | PI3K Ca | P13K Ca | auguuuacuaccaaaugga | 619 |
| | | 1 | | |
| NM_001047 | SRD5A1 | SRD5A1 | uaacugcagccaacuauuu | 620 |
| | | 2 | | |
| NM_030662 | MAP2K2 | MAP2K2 | uccaggaguuugucaauaa | 621 |
| | | 3 | | |
| NM_001047 | SRD5A1 | SRD5A1 | gcagauacuugagccauug | 622 |
| | | 1 | | |
| NM_001047 | SRD5A1 | SRD5A1 | ccggaaauuugaagaguau | 623 |
| | | 4 | | |
| NM_000348 | SRD5A2 | SRD5A2 | ggacauuuguguacucacu | 624 |
| | | 3 | | |
| NM_005508 | CCR4 | CHD4 | gaaagcauauacagcaauu | 625 |
| NM_002605 | PDE8A | PDE8A | caaagaagauaaccaaugu | 626 |
| NM_000455 | STK11 | STK11 | gaaacauccuccggcugaa | 627 |

As shown in FIG. 51, when eight separate unmodified, toxic siRNA (MAP2K2 d3, SRD5A1 d1, SRD5A1 d2, SRD5A1 d4, SRD5A2 d3, PDE8A, STK11, and CHD4) were transfected into cells, each decreased cell viability below 75%. In contrast, chemical modification of all eight duplexes markedly decreased siRNA-induced toxicity without significantly altering target specific knockdown. Taken together, these findings strongly suggest that the described sequence-specific toxicity is the result of RNAi dependent off-target gene modulation.

### Example 5

### The AUUUA and AUUUG Motifs: Cell Death by Apoptosis

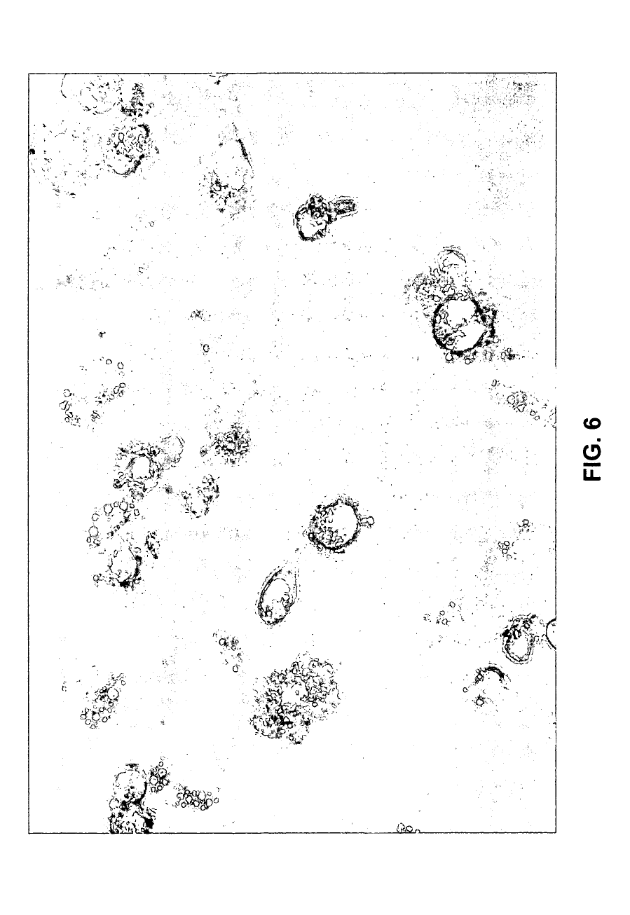

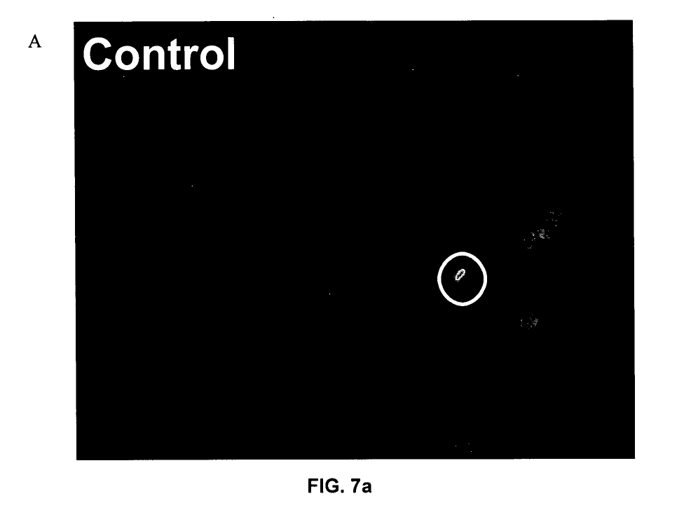

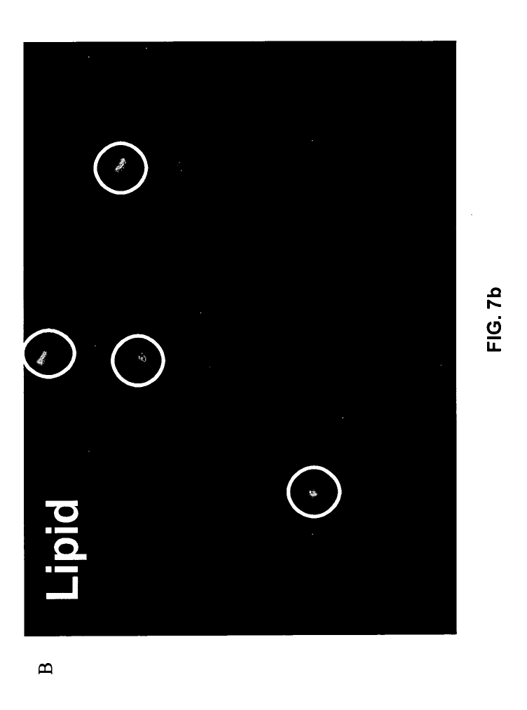

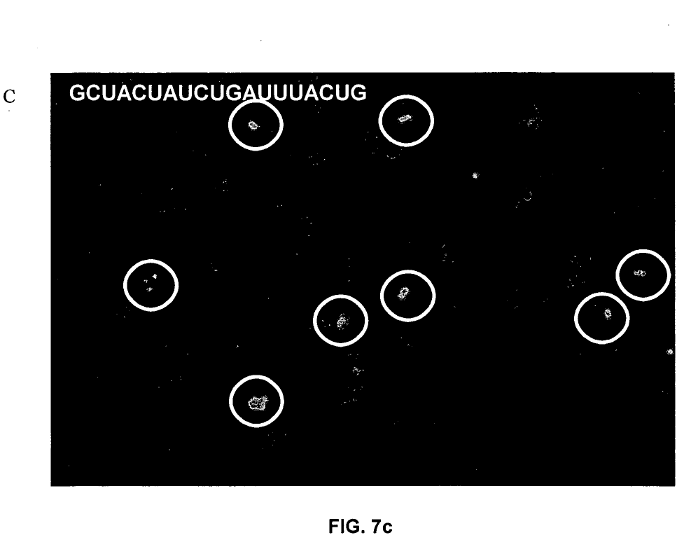

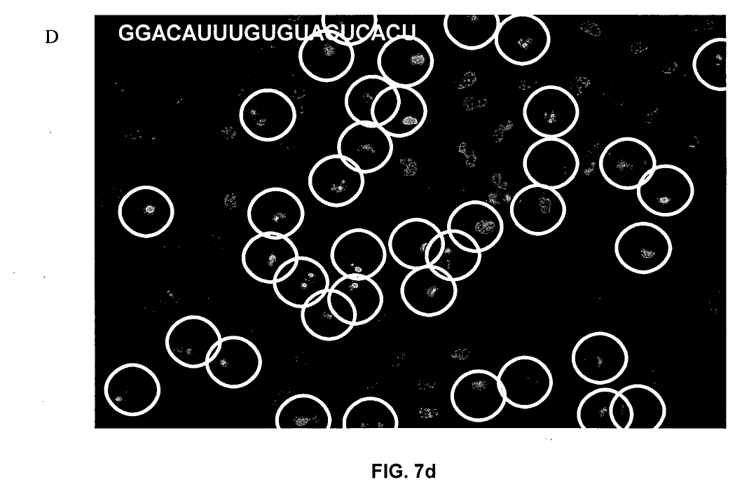

To better understand the mechanism behind the toxic effects of the AUUUA and AUUUG motifs, cells transfected with siRNA containing these sequences were examined by microscopy. Specifically, HeLa cells transfected with either GGACAUUUGUGUACUCACU (SEQ. ID NO.640) or GCUACUAUCUGAUUUACUG (sense strand) (SEQ. ID NO.641) siRNA were cultured for 48 hours and then examined by phase contrast microscopy. Additionally, cells were stained with Hoechst 33342 (2 micrograms/ml, 30 minutes, 37° C.) and examined by fluorescence microscopy. FIG. 6 shows a phase contrast micrograph of HeLa cells transfected with the GGACAUUUGUGUACUCACU sequence. A large number of the cells present in this culture have released from the solid support and exhibit a rounded or “balled-up” phenotype typical of cells undergoing apoptosis. FIGS. 7C and D support the hypothesis that AUUUA and AUUUG motifs induce apoptosis. Cells transfected with the GGACAUUUGUGUACUCACU (SEQ. ID NO.642) or GCUACUAUCUGAUUUACUG (SEQ. ID NO.643) siRNA and stained with Hoechst 33342 exhibit condensed nuclei, a phenotype that is indicative of apoptosis (See FIGS. 7A and 7B, controls, for comparison). Furthermore, the number of cells exhibiting the condensed nuclei phenotype is greater in cultures transfected with GGACAUUUGUGUACUCACU (SEQ. ID NO.644) sequence than with sequences transfected with the GCUACUAUCUGAUUUACUG (SEQ. ID NO.645) sequence. This observation lends further support to previous experiments (FIG. 5) that demonstrated that A/G UUU A/G/U motifs located in the 5′ portion of the sense strand exhibited higher levels of toxicity than equivalent/related motifs located in the 3′ half of the sense strand.

### Example 6

### Induction of Cell Death by Toxic Motifs in Multiple Cell Types

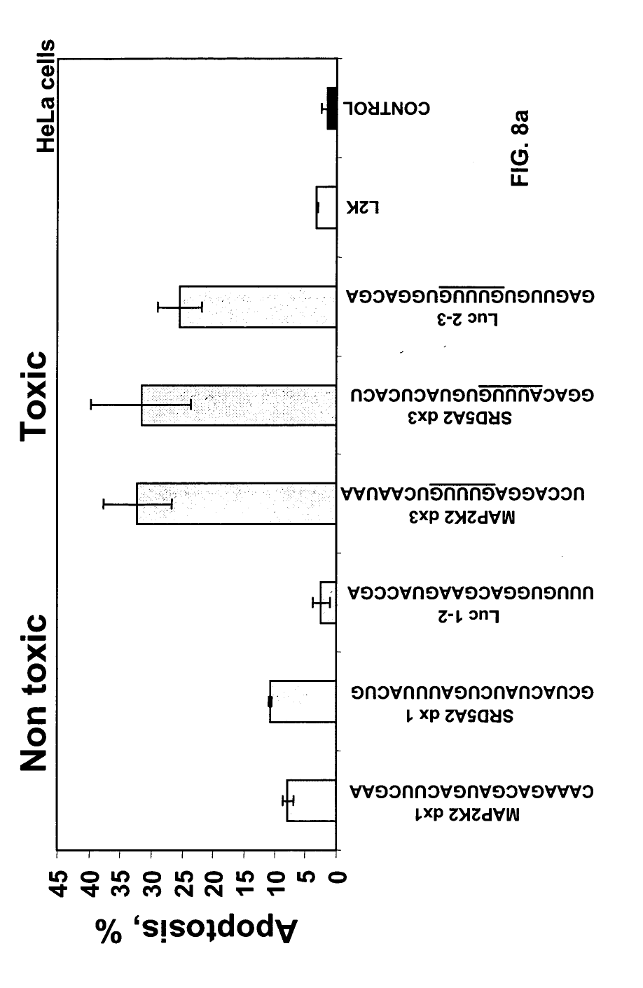

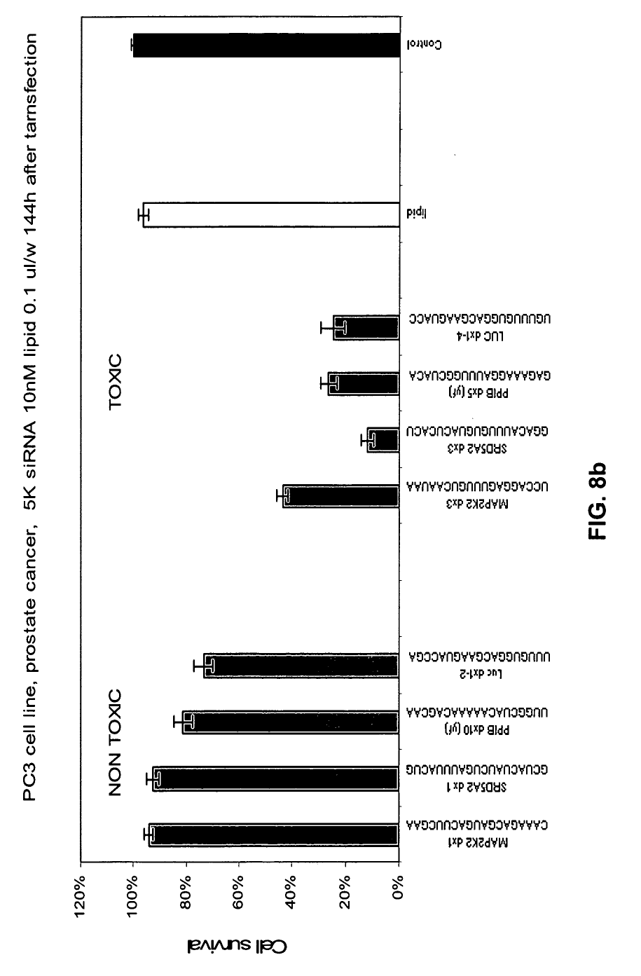

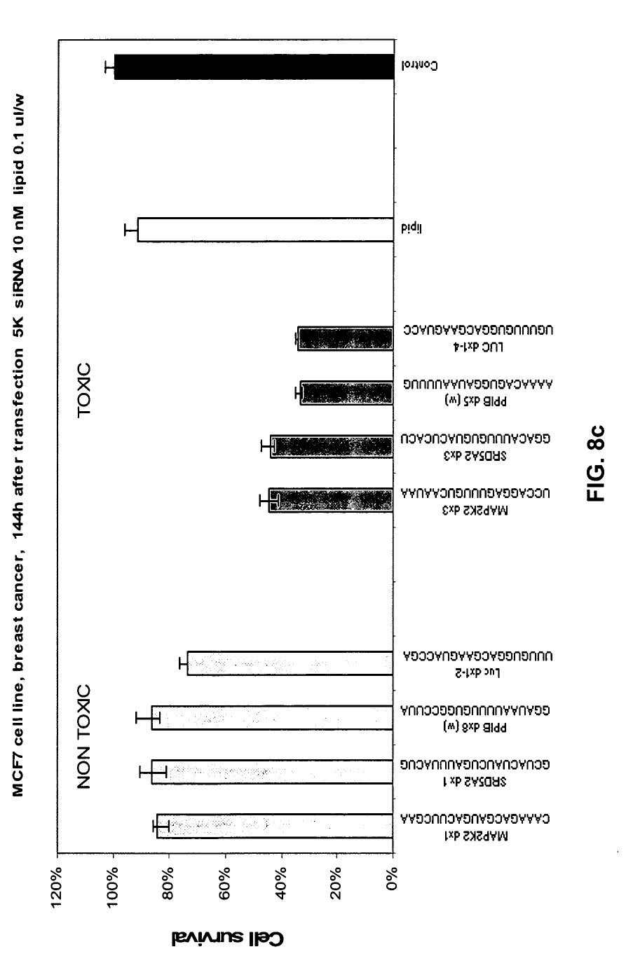

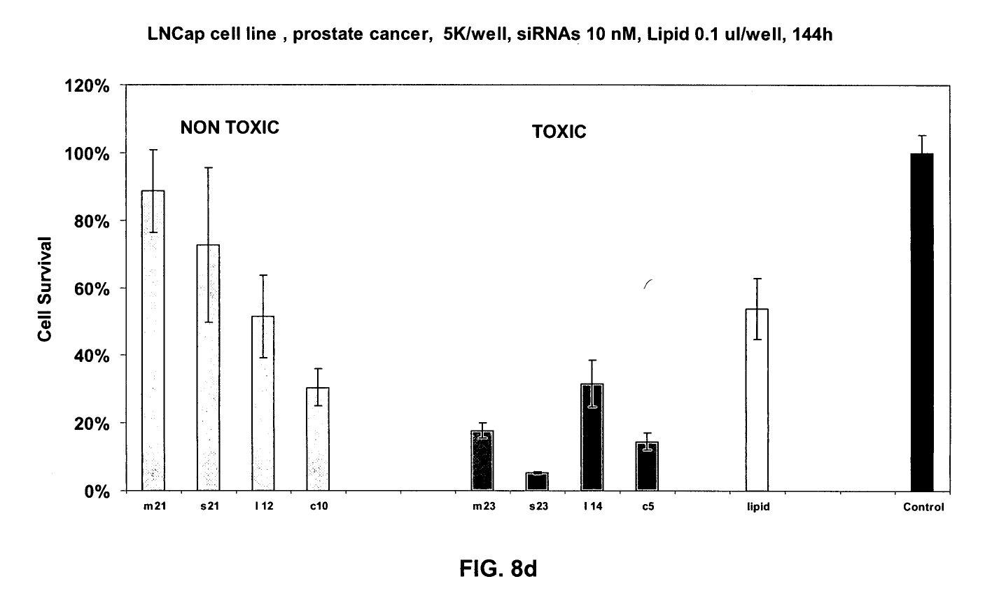

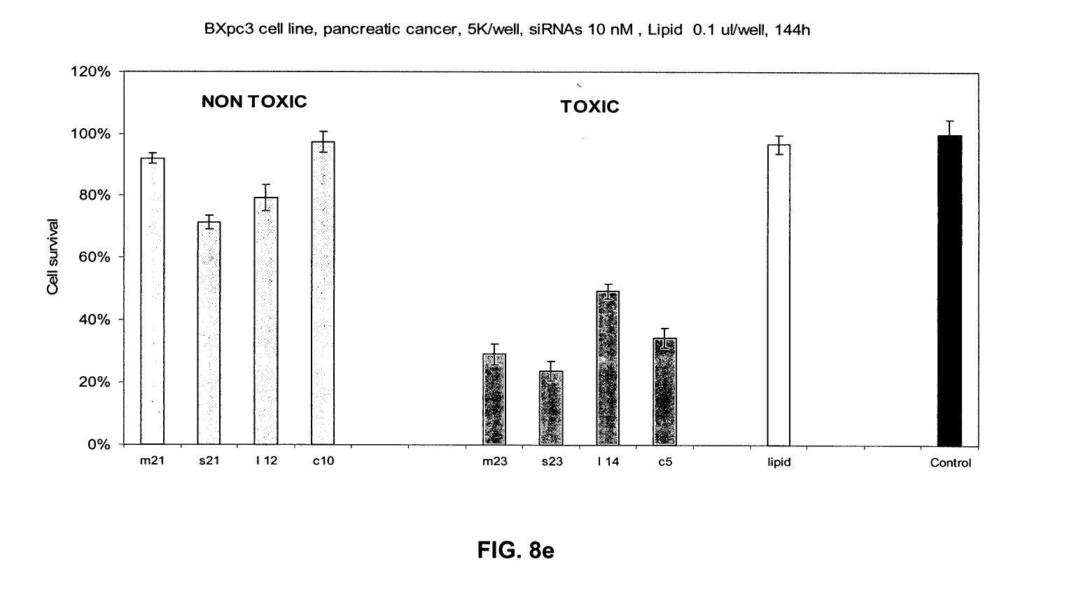

To determine the cell specificity of siRNA containing toxic motifs, multiple cell types were transfected with siRNA containing toxic motifs. Specifically, HeLa cells, PC3 cells, MCF7 cells, LnCap cells, and BXPC3 cells (ATCC, Manassas, Va.) were plated (5,000 cells per well) and transfected (10 nanomolar, Lipofectamine 2000) with non-toxic (e.g., MAP2K2-1, SRD5A2-1, PPIB-dx8, PPIB-dx10 and Luciferase 1-2 and toxic (e.g. MAP2K2-3, SRD5A2-3, PPIB-dx5 and Luciferase 2-3 (5′ GAGUUGUGUUUGUGGACGA, sense strand (SEQ. ID NO.646)) siRNA and examined for the induction of cell death using the Alamar Blue assay. Results (FIGS. 8a-e) show that toxic sequences induced a lethal phenotype in all cell types tested, suggesting that the target of such sequences is found in a diverse set of cell types. The sequences for each of these siRNAs are listed in Table 1 or the associated Figure legend.

### Example 7

### Toxic Motifs and Sensitization

Experiments were performed to determine whether introduction of toxic motifs could sensitize cells to other agents that induced apoptosis. Specifically, HeLa cells were plated and transfected as in Example 1. At t=24 hours, the media was replaced with media that contained 200 micromolar hydrogen peroxide (H₂O₂). The 200 micromolar hydrogen peroxide has previously been shown to be non-toxic to HeLa cells. Subsequently, cell survival was measured 24 hours later using the Alamar Blue Assay.

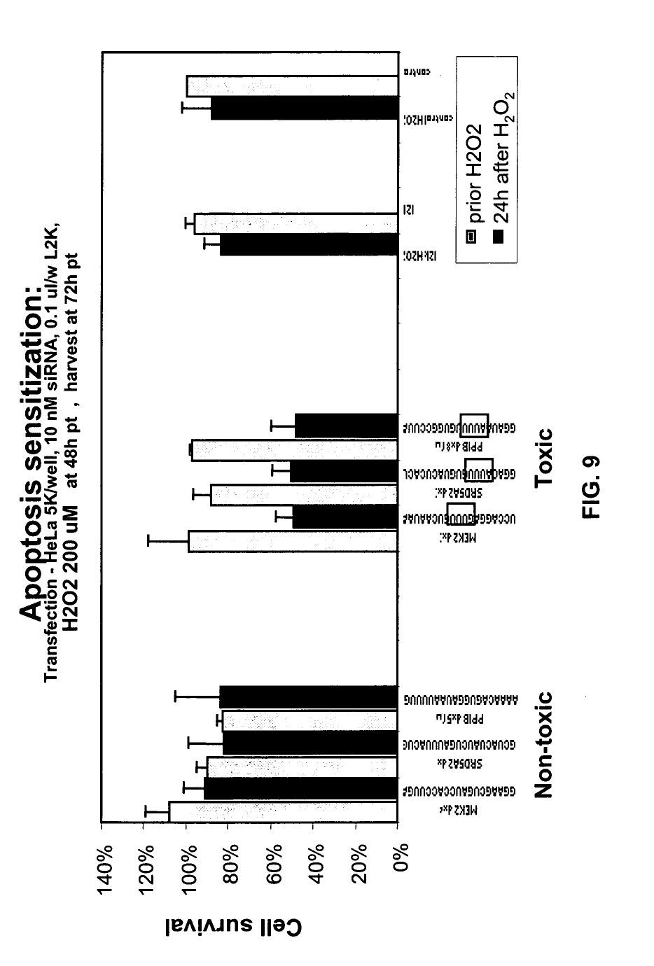

Results of these experiments are reported in FIG. 9. Treatment of cells with hydrogen peroxide alone had little or no effect on the viability of the cells at day 2. Similarly, addition of hydrogen peroxide to cells that had been transfected with non-toxic sequences (MAP2K2-4, SRD5A2-1, and PPIB-dx 5) did not significantly alter the level of cell death over cells that had been transfected with non-toxic sequences alone. In contrast, addition of H₂O₂ to cells that had been transfected with siRNA containing toxic sequences (MAP2K2-3 SRD5A2-3, and PPIP-dx8) led to heightened levels of cell death. Alone, each of the above sequences induced 20% or less toxicity on day 2. When combined with non-toxic levels of H₂O₂ the level of toxicity rose to >50%. Thus, the toxic sequences heightened the sensitivity of the cells to the presence of H₂O₂.

Effects of Non-Specific siRNA Containing the GCCA Motif

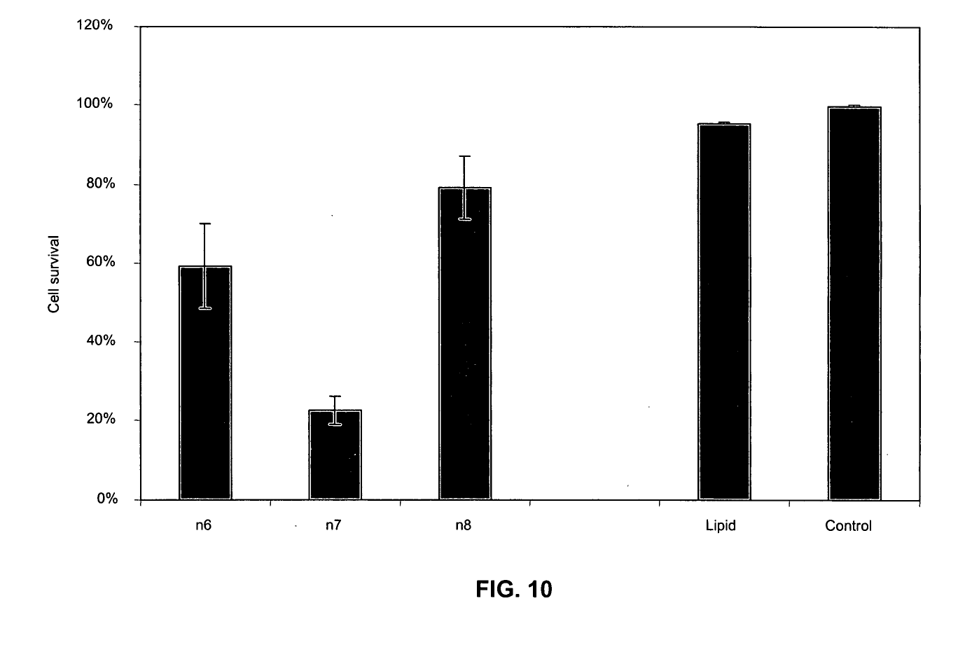

Two non-specific sequences (i.e., sequences that are not designed to target a particular gene via the RNAi pathway), ACUCUAUCGCCAGCGUGAC and ACUCUAGCGCCAUCGUGCC, (SEQ. ID NO.647) both containing the GCCA motif were transfected into HeLa cells (10 nanomolar, Lipofectamine 2000) and tested for toxicity using the Alamar Blue assay at t=72 hours. Results showed (FIG. 10) that a non-specific sequence that does not contain the GCCA motif induces low amounts of cell death (80% cell survival). In contrast, both sequences that contain the GCCA motif induced greater levels of toxicity (40% and 80% cell death for n6 and n7 respectively). Potential targets for the GCCA motif include members of the nuclear factor I family (see, for example, Bachurski, C. J. et al., (1997) “Nuclear Factor I Family Members Regulate the Transcription of Surfactant Protein-C” J. Biol. Chem., 272 (52): 32759-32766; Gronostajski R. M., (1987) “Site-specific DNA binding of nuclear factor I: effect of the spacer region.” Nucleic Acids Res. 15(14):5545-59).

### Example 8

### Toxic Motifs and Transfection Control

To develop an siRNA that can be used as a transfection control reagent, the sequence of the Eg5 gene (NM_{—}004523.2), also known as Kinesin family member 11 or TRIP5) was scanned to identify a sequence that contained one or more toxic motifs. A 62 base pair sequence (called Eg5-tox) containing two toxic motifs (sense, AUUUU and antisense, GCCA) was identified:

Sense strand:

(SEQ. ID NO. 648)

5′auuuucaaga cuucauugac aguggccgau aagauagaag
aucaaaaaaa ggaacuagau ggdtdt3′

Antisense strand:

(SEQ. ID NO.6 49)

5′ccaucuaguu ccuuuuuuug aucuucuauc uuaucggcca
cugucaauga agucuugaaa audtdt3′

To test the ability of this 62 base-pair sequence to induce cell death, HeLa cells were plated at a density of 10,000 cells per well (96-well plate) and transfected with the Eg5-tox sequence at varying concentrations (0.5-200 nanomolar) using Lipofectamine 2000. Subsequently, cells were cultured over the course of 72 hours and assessed for cell viability by staining with Hoechst 33342 dye. Results of these experiments showed that transfection of the Eg5-tox duplexes induced significant levels of cell death. Transfections at 50 nanomolar and 12 nanomolar concentrations were sufficient to induce greater than 90% cell death within 24 and 48 hours, respectively.

To further assess the effects of toxic molecules as transfection controls under these conditions, successively smaller molecules were tested for the ability to induce cell death. Specifically, Eg5-tox duplexes that were used are described in the table below:

TABLE XI

EXAMPLE 5 TOXIC DUPLEXES

| | | |
|---|---|---|
| | | SEQ. |
| | LENGTH | ID |
| sIRNA SEQUENCE (SENSE, 5′→3′) | (BASEPAIRS) | NO. |

| | | |
|---|---|---|
| auuuucaaga cuucauugac aguggccgau | 62 | 628 |
| aagauagaag aucaaaaaaa ggaacuagau | | |
| ggdtdt | | |
| auuuucaaga cuucauugac aguggccgau | 57 | 629 |
| aagauagaag aucaaaaaaa ggaacuadtdt | | |
| auuuucaaga cuucauugac aguggccgau | 52 | 630 |
| aagauagaag aucaaaaaaa ggdtdt | | |
| auuuucaaga cuucauugac aguggccgau | 47 | 631 |
| aagauagaag aucaaaadtdt | | |
| auuuucaaga cuucauugac aguggccgau | 42 | 632 |
| aagauagaag audtdt | | |
| auuuucaaga cuucauugac aguggccgau | 37 | 633 |
| aagauagdtdt | | |
| auuuucaaga cuucauugac aguggccgau | 32 | 634 |
| auuuucaaga cuucauugac aguggccdtdt | 27 | 635 |
| auuuucaaga cuucauugac agdtdt | 22 | 636 |
| auuuucaaga cuucauugadt dt | 19 | 637 |

The duplexes described in the above table contained at least one toxic motif, and were introduced into HeLa cells using the previously described conditions and assessed for the ability to induce cell death. Results of these experiment established that all of the fragments with the exception of the smallest duplex (21 bp) were capable of inducing cell death. Small siRNAs or pools of siRNA that did not contain toxic motifs, but did down regulate the Eg5 target, did not induce cell death in these time frames (144 hours).

While the invention has been described in connection with specific embodiments thereof, it will be understood that it is capable of further modifications and this application is intended to cover any variations, uses, or adaptations of the invention following, in general, the principles of the invention and including such departure from the present disclosure as come within known or customary practice within the art to which the invention pertains and as may be applied to the essential features hereinbefore set forth and as follows in the scope of the appended claims.

1. A unimolecular polynucleotide, comprising at least one toxicity region comprising a sequence selected from the group consisting of GUUU, AGCA, GCAC, CUGG, AGAC, UGGC, NUUU, wherein N is any nucleotide, or a complement of any of the foregoing, wherein said unimolecular polynucleotide is capable of forming an intramolecular duplex of 5 or more base pairs, and wherein said duplex comprises a sense region and an antisense region that are at least substantially complementary.

2. The unimolecular polynucleotide of claim 1, wherein said at least one toxicity region is within said sense region.

3. The unimolecular polynucleotide of claim 1, wherein said at least one toxicity region is within said antisense region.

4. The unimolecular polynucleotide of claim 1, wherein said antisense region comprises from 19 to 40 bases.

5. A double stranded polynucleotide, comprising at least one toxicity region comprising a sequence selected from the group consisting of GUUU, AGCA, GCAC, CUGG, AGAC, UGGC, NUUU, wherein N is any nucleotide, or a complement of any of the foregoing, wherein said polynucleotide is capable of forming a duplex of 5 or more base pairs, and wherein said duplex comprises a sense strand and an antisense strand that are at least substantially complementary.

6. The double stranded polynucleotide of claim 5, wherein said at least one toxicity region is within said sense region.

7. The double stranded polynucleotide of claim 5, wherein said at least one toxicity region is within said antisense region.

8. The double stranded polynucleotide of claim 5, wherein said antisense region comprises from 19 to 40 bases.

9. An RNA duplex for inducing a toxic response in a cell, comprising a nucleotide sequence GUUU, AGCA, GCAC, CUGG, AGAC, UGGC, NUUU, wherein N is any nucleotide, or a complement of any of the foregoing, wherein said RNA duplex is at least 5 base pairs in length, and wherein said RNA duplex comprises at least two regions that are at least substantially complementary.

10. The RNA duplex according to claim 9, wherein said RNA duplex comprises two separate strands.

11. The RNA duplex to claim 9, wherein said RNA duplex is a unimolecular siRNA.

12. A method of inducing a toxic response in a cell, comprising introducing into the cell a unimolecular polynucleotide or a double stranded polynucleotide, wherein said unimolecular polynucleotide or double stranded polynucleotide comprises at least one toxicity region comprising a sequence selected from the group consisting of GUUU, AGCA, GCAC, CUGG, AGAC, UGGC, NUUU, wherein N is any nucleotide, or a complement of any of the foregoing, wherein said unimolecular polynucleotide or double stranded polynucleotide comprises a duplex region of 5 or more base pairs, and wherein said unimolecular polynucleotide or double stranded polynucleotide comprises a sense region and an antisense region that are at least substantially complementary.

13. The method according to claim 12, wherein said at least one toxicity region is within said sense region.

14. The method according to claim 12, wherein said at least one toxicity region is within said antisense region.

15. The method according to claim 12, further comprising exposing said cell to at least one agent or condition that stresses said cell.

16. The method according to claim 12, wherein said toxicity region induces apoptosis in said cell.

17. The method according to claim 15, wherein said at least one agent that stresses said cell comprises a peroxide.

18. The method according to claim 17, wherein the peroxide is hydrogen peroxide.

19. A method for screening a library of nucleic acids for a toxicity region, comprising screening a database containing nucleic acid sequences and identifying those sequences that contain toxic motifs.

20. A transfection control method, comprising:
(a) transfecting a first group of cells with one or more polynucleotides or double-stranded polynucleotides;
(b) transfecting a second group of cells with a duplex RNA, wherein said duplex comprises at least one toxicity region comprising a sequence selected from the group consisting of GUUU, AGCA, GCAC, CUGG, AGAC, UGGC, NUUU, wherein N is any nucleotide, or a complement of any of the foregoing, wherein said duplex is greater than 5 base pairs in length, and wherein said duplex comprises a sense region and an antisense region that are at least substantially complementary, and wherein said first and said second cells are transfected under similar conditions;
(c) maintaining said first and said second groups of cells under conditions sufficient for cell growth; and,
(d) determining the level of cell viability in said second group of cells.

21. The method according to claim 20, wherein said RNA duplex is 19-40 base pairs in length.

22. The method according to claim 20, wherein said RNA duplex greater than 40 base pairs in length.

23. The method according to claim 20, wherein said RNA duplex is greater than 64 base pairs in length.

24. The method according to claim 20, wherein said duplex is an siRNA and targets an essential gene.

25. The method according to claim 12, wherein the unimolecular polynucleotide or double stranded polynucleotide induces a toxic response in the cell through an off-target effect.


SEQUENCE LISTING (658 sequences)

SEQ ID NO: 1 - RNA, 4 nt, Artificial Sequence
  Synthetic polynucleotide
guuu

SEQ ID NO: 2 - RNA, 4 nt, Artificial Sequence
  Synthetic polynucleotide
agca

SEQ ID NO: 3 - RNA, 4 nt, Artificial Sequence
  Synthetic polynucleotide
gcac

SEQ ID NO: 4 - RNA, 4 nt, Artificial Sequence
  Synthetic polynucleotide
cugg

SEQ ID NO: 5 - RNA, 4 nt, Artificial Sequence
  Synthetic polynucleotide
agac

SEQ ID NO: 6 - RNA, 4 nt, Artificial Sequence
  Synthetic polynucleotide
uggc

SEQ ID NO: 7 - RNA, 4 nt, Artificial Sequence
  Synthetic polynucleotide
nuuu

SEQ ID NO: 8 - RNA, 3 nt, Artificial Sequence
  Synthetic polynucleotide
uuu

SEQ ID NO: 9 - RNA, 3 nt, Artificial Sequence
  Synthetic polynucleotide
aaa

SEQ ID NO: 10 - RNA, 4 nt, Artificial Sequence
  Synthetic polynucleotide
gcca

SEQ ID NO: 11 - RNA, 4 nt, Artificial Sequence
  Synthetic polynucleotide
uggc

SEQ ID NO: 12 - RNA, 19 nt, Artificial Sequence
  Synthetic polynucleotide
ggacauuuguguacucacu

SEQ ID NO: 13 - RNA, 19 nt, Artificial Sequence
  Synthetic polynucleotide
gcuacuaucugauuuacug

SEQ ID NO: 14 - RNA, 19 nt, Artificial Sequence
  Synthetic polynucleotide
uuuguggacgaaguaccga

SEQ ID NO: 15 - RNA, 19 nt, Artificial Sequence
  Synthetic polynucleotide
uguuuguggacgaaguacc

SEQ ID NO: 16 - RNA, 19 nt, Artificial Sequence
  Synthetic polynucleotide
gaguuguguuuguggacga

SEQ ID NO: 17 - RNA, 19 nt, Artificial Sequence
  Synthetic polynucleotide
uuggcuacaaaaacagcaa

SEQ ID NO: 18 - RNA, 19 nt, Artificial Sequence
  Synthetic polynucleotide
aaaacaguggauaauuuug

SEQ ID NO: 19 - RNA, 19 nt, Artificial Sequence
  Synthetic polynucleotide
ggauaauuuuguggccuua

SEQ ID NO: 20 - RNA, 19 nt, Artificial Sequence
  Synthetic polynucleotide
acucuagcgccaucgugcc

SEQ ID NO: 21 - RNA, 19 nt, Artificial Sequence
  Synthetic polynucleotide
acucuaucgccagcgugac

SEQ ID NO: 22 - RNA, 19 nt, Artificial Sequence
  Synthetic polynucleotide
acucuaucugcacgcugac

SEQ ID NO: 23 - RNA, 19 nt, Artificial Sequence
  Synthetic polynucleotide
ccaaagcucuggacuuauu

SEQ ID NO: 24 - RNA, 19 nt, Artificial Sequence
  Synthetic polynucleotide
aaacagaucuuuacaagcu

SEQ ID NO: 25 - RNA, 19 nt, Artificial Sequence
  Synthetic polynucleotide
caagaggauugaaguagaa

SEQ ID NO: 26 - RNA, 19 nt, Artificial Sequence
  Synthetic polynucleotide
guacagggcuccagaaauu

SEQ ID NO: 27 - RNA, 19 nt, Artificial Sequence
  Synthetic polynucleotide
gaccggauguuaaccuuua

SEQ ID NO: 28 - RNA, 19 nt, Artificial Sequence
  Synthetic polynucleotide
agacugaccuguacaaguu

SEQ ID NO: 29 - RNA, 19 nt, Artificial Sequence
  Synthetic polynucleotide
gaaacuaccuacagucucu

SEQ ID NO: 30 - RNA, 19 nt, Artificial Sequence
  Synthetic polynucleotide
gcuacacgcaguugcagua

SEQ ID NO: 31 - RNA, 19 nt, Artificial Sequence
  Synthetic polynucleotide
ggaacucgaucguaucauu

SEQ ID NO: 32 - RNA, 19 nt, Artificial Sequence
  Synthetic polynucleotide
caagggagguuacaccaaa

SEQ ID NO: 33 - RNA, 19 nt, Artificial Sequence
  Synthetic polynucleotide
ucaaggaacucgaucguau

SEQ ID NO: 34 - RNA, 19 nt, Artificial Sequence
  Synthetic polynucleotide
gaaaugauugcacuauuga

SEQ ID NO: 35 - RNA, 19 nt, Artificial Sequence
  Synthetic polynucleotide
gcuacuaucugauuuacug

SEQ ID NO: 36 - RNA, 19 nt, Artificial Sequence
  Synthetic polynucleotide
gcuaugcccuggccacuug

SEQ ID NO: 37 - RNA, 19 nt, Artificial Sequence
  Synthetic polynucleotide
ggacauuuguguacucacu

SEQ ID NO: 38 - RNA, 19 nt, Artificial Sequence
  Synthetic polynucleotide
uugggugucuucuuauuua

SEQ ID NO: 39 - RNA, 19 nt, Artificial Sequence
  Synthetic polynucleotide
gcagauacuugagccauug

SEQ ID NO: 40 - RNA, 19 nt, Artificial Sequence
  Synthetic polynucleotide
uaacugcagccaacuauuu

SEQ ID NO: 41 - RNA, 19 nt, Artificial Sequence
  Synthetic polynucleotide
gaaagccuaugccacuguu

SEQ ID NO: 42 - RNA, 19 nt, Artificial Sequence
  Synthetic polynucleotide
ccggaaauuugaagaguau

SEQ ID NO: 43 - RNA, 19 nt, Artificial Sequence
  Synthetic polynucleotide
auguuuacuaccaaaugga

SEQ ID NO: 44 - RNA, 19 nt, Artificial Sequence
  Synthetic polynucleotide
aacuagaaguauguugcua

SEQ ID NO: 45 - RNA, 19 nt, Artificial Sequence
  Synthetic polynucleotide
aauggcuuugaaucuuugg

SEQ ID NO: 46 - RNA, 19 nt, Artificial Sequence
  Synthetic polynucleotide
cugaagaaagcauugacua

SEQ ID NO: 47 - RNA, 19 nt, Artificial Sequence
  Synthetic polynucleotide
cgacaagacugccgagaga

SEQ ID NO: 48 - RNA, 19 nt, Artificial Sequence
  Synthetic polynucleotide
ucaagugucuccuaauaug

SEQ ID NO: 49 - RNA, 19 nt, Artificial Sequence
  Synthetic polynucleotide
ggauucaguuggagugauu

SEQ ID NO: 50 - RNA, 19 nt, Artificial Sequence
  Synthetic polynucleotide
uuucaagugucuccuaaua

SEQ ID NO: 51 - RNA, 19 nt, Artificial Sequence
  Synthetic polynucleotide
gggagauagugaugaagua

SEQ ID NO: 52 - RNA, 19 nt, Artificial Sequence
  Synthetic polynucleotide
gaaguacauccauuauaag

SEQ ID NO: 53 - RNA, 19 nt, Artificial Sequence
  Synthetic polynucleotide
guacgacaaccgggagaua

SEQ ID NO: 54 - RNA, 19 nt, Artificial Sequence
  Synthetic polynucleotide
agauagugaugaaguacau

SEQ ID NO: 55 - RNA, 19 nt, Artificial Sequence
  Synthetic polynucleotide
gaacaccgagugccaagaa

SEQ ID NO: 56 - RNA, 19 nt, Artificial Sequence
  Synthetic polynucleotide
gagccuuacugccuuugua

SEQ ID NO: 57 - RNA, 19 nt, Artificial Sequence
  Synthetic polynucleotide
ggccggaggcgcuuuacua

SEQ ID NO: 58 - RNA, 19 nt, Artificial Sequence
  Synthetic polynucleotide
ucgacgcaguggacauuaa

SEQ ID NO: 59 - RNA, 19 nt, Artificial Sequence
  Synthetic polynucleotide
gcacggagauguugcagua

SEQ ID NO: 60 - RNA, 19 nt, Artificial Sequence
  Synthetic polynucleotide
gcaaagaacaucauccaua

SEQ ID NO: 61 - RNA, 19 nt, Artificial Sequence
  Synthetic polynucleotide
gacaugaaauccaacaaua

SEQ ID NO: 62 - RNA, 19 nt, Artificial Sequence
  Synthetic polynucleotide
caaagaacaucauccauag

SEQ ID NO: 63 - RNA, 19 nt, Artificial Sequence
  Synthetic polynucleotide
gcacauggauggagguucu

SEQ ID NO: 64 - RNA, 19 nt, Artificial Sequence
  Synthetic polynucleotide
gcagagagagcagauuuga

SEQ ID NO: 65 - RNA, 19 nt, Artificial Sequence
  Synthetic polynucleotide
gagguucucuggaucaagu

SEQ ID NO: 66 - RNA, 19 nt, Artificial Sequence
  Synthetic polynucleotide
gagcagauuugaagcaacu

SEQ ID NO: 67 - RNA, 19 nt, Artificial Sequence
  Synthetic polynucleotide
caaagacgaugacuucgaa

SEQ ID NO: 68 - RNA, 19 nt, Artificial Sequence
  Synthetic polynucleotide
gaucagcauuugcauggaa

SEQ ID NO: 69 - RNA, 19 nt, Artificial Sequence
  Synthetic polynucleotide
uccaggaguuugucaauaa

SEQ ID NO: 70 - RNA, 19 nt, Artificial Sequence
  Synthetic polynucleotide
ggaagcugauccaccuuga

SEQ ID NO: 71 - RNA, 19 nt, Artificial Sequence
  Synthetic polynucleotide
gaaguacauccauuauaag

SEQ ID NO: 72 - RNA, 19 nt, Artificial Sequence
  Synthetic polynucleotide
aaacagaucuuuacaagcu

SEQ ID NO: 73 - RNA, 19 nt, Artificial Sequence
  Synthetic polynucleotide
uuucaagugucuccuaaua

SEQ ID NO: 74 - RNA, 19 nt, Artificial Sequence
  Synthetic polynucleotide
gcaaagaacaucauccaua

SEQ ID NO: 75 - RNA, 19 nt, Artificial Sequence
  Synthetic polynucleotide
gcagagagagcagauuuga

SEQ ID NO: 76 - RNA, 19 nt, Artificial Sequence
  Synthetic polynucleotide
ucaaggaacucgaucguau

SEQ ID NO: 77 - RNA, 19 nt, Artificial Sequence
  Synthetic polynucleotide
gacaugaaauccaacaaua

SEQ ID NO: 78 - RNA, 19 nt, Artificial Sequence
  Synthetic polynucleotide
ucaagugucuccuaauaug

SEQ ID NO: 79 - RNA, 19 nt, Artificial Sequence
  Synthetic polynucleotide
caaagacgaugacuucgaa

SEQ ID NO: 80 - RNA, 19 nt, Artificial Sequence
  Synthetic polynucleotide
ggaagcugauccaccuuga

SEQ ID NO: 81 - RNA, 19 nt, Artificial Sequence
  Synthetic polynucleotide
caagaggauugaaguagaa

SEQ ID NO: 82 - RNA, 19 nt, Artificial Sequence
  Synthetic polynucleotide
ccaaagcucuggacuuauu

SEQ ID NO: 83 - RNA, 19 nt, Artificial Sequence
  Synthetic polynucleotide
guacgacaaccgggagaua

SEQ ID NO: 84 - RNA, 19 nt, Artificial Sequence
  Synthetic polynucleotide
agauagugaugaaguacau

SEQ ID NO: 85 - RNA, 19 nt, Artificial Sequence
  Synthetic polynucleotide
gggagauagugaugaagua

SEQ ID NO: 86 - RNA, 19 nt, Artificial Sequence
  Synthetic polynucleotide
gaaaugauugcacuauuga

SEQ ID NO: 87 - RNA, 19 nt, Artificial Sequence
  Synthetic polynucleotide
gcacggagauguugcagua

SEQ ID NO: 88 - RNA, 19 nt, Artificial Sequence
  Synthetic polynucleotide
gcuacuaucugauuuacug

SEQ ID NO: 89 - RNA, 19 nt, Artificial Sequence
  Synthetic polynucleotide
caagggagguuacaccaaa

SEQ ID NO: 90 - RNA, 19 nt, Artificial Sequence
  Synthetic polynucleotide
gagguucucuggaucaagu

SEQ ID NO: 91 - RNA, 19 nt, Artificial Sequence
  Synthetic polynucleotide
gcuacacgcaguugcagua

SEQ ID NO: 92 - RNA, 19 nt, Artificial Sequence
  Synthetic polynucleotide
agacugaccuguacaaguu

SEQ ID NO: 93 - RNA, 19 nt, Artificial Sequence
  Synthetic polynucleotide
gaucagcauuugcauggaa

SEQ ID NO: 94 - RNA, 19 nt, Artificial Sequence
  Synthetic polynucleotide
ggaacucgaucguaucauu

SEQ ID NO: 95 - RNA, 19 nt, Artificial Sequence
  Synthetic polynucleotide
cgacaagacugccgagaga

SEQ ID NO: 96 - RNA, 19 nt, Artificial Sequence
  Synthetic polynucleotide
gagccuuacugccuuugua

SEQ ID NO: 97 - RNA, 19 nt, Artificial Sequence
  Synthetic polynucleotide
cugaagaaagcauugacua

SEQ ID NO: 98 - RNA, 19 nt, Artificial Sequence
  Synthetic polynucleotide
ggccggaggcgcuuuacua

SEQ ID NO: 99 - RNA, 19 nt, Artificial Sequence
  Synthetic polynucleotide
guacagggcuccagaaauu

SEQ ID NO: 100 - RNA, 19 nt, Artificial Sequence
  Synthetic polynucleotide
ucgacgcaguggacauuaa

SEQ ID NO: 101 - RNA, 19 nt, Artificial Sequence
  Synthetic polynucleotide
aacuagaaguauguugcua

SEQ ID NO: 102 - RNA, 19 nt, Artificial Sequence
  Synthetic polynucleotide
gaccggauguuaaccuuua

SEQ ID NO: 103 - RNA, 19 nt, Artificial Sequence
  Synthetic polynucleotide
uugggugucuucuuauuua

SEQ ID NO: 104 - RNA, 19 nt, Artificial Sequence
  Synthetic polynucleotide
aauggcuuugaaucuuugg

SEQ ID NO: 105 - RNA, 19 nt, Artificial Sequence
  Synthetic polynucleotide
gcacauggauggagguucu

SEQ ID NO: 106 - RNA, 19 nt, Artificial Sequence
  Synthetic polynucleotide
ggauucaguuggagugauu

SEQ ID NO: 107 - RNA, 19 nt, Artificial Sequence
  Synthetic polynucleotide
caaagaacaucauccauag

SEQ ID NO: 108 - RNA, 19 nt, Artificial Sequence
  Synthetic polynucleotide
gaaagccuaugccacuguu

SEQ ID NO: 109 - RNA, 19 nt, Artificial Sequence
  Synthetic polynucleotide
gcuaugcccuggccacuug

SEQ ID NO: 110 - RNA, 19 nt, Artificial Sequence
  Synthetic polynucleotide
gagcagauuugaagcaacu

SEQ ID NO: 111 - RNA, 19 nt, Artificial Sequence
  Synthetic polynucleotide
uaacugcagccaacuauuu

SEQ ID NO: 112 - RNA, 19 nt, Artificial Sequence
  Synthetic polynucleotide
auguuuacuaccaaaugga

SEQ ID NO: 113 - RNA, 19 nt, Artificial Sequence
  Synthetic polynucleotide
uccaggaguuugucaauaa

SEQ ID NO: 114 - RNA, 19 nt, Artificial Sequence
  Synthetic polynucleotide
gcagauacuugagccauug

SEQ ID NO: 115 - RNA, 19 nt, Artificial Sequence
  Synthetic polynucleotide
gaaacuaccuacagucucu

SEQ ID NO: 116 - RNA, 19 nt, Artificial Sequence
  Synthetic polynucleotide
ccggaaauuugaagaguau

SEQ ID NO: 117 - RNA, 19 nt, Artificial Sequence
  Synthetic polynucleotide
gaacaccgagugccaagaa

SEQ ID NO: 118 - RNA, 19 nt, Artificial Sequence
  Synthetic polynucleotide
ggacauuuguguacucacu

SEQ ID NO: 119 - RNA, 19 nt, Artificial Sequence
  Synthetic polynucleotide
acgggcaaggccaaguggg

SEQ ID NO: 120 - RNA, 19 nt, Artificial Sequence
  Synthetic polynucleotide
cgggcaaggccaaguggga

SEQ ID NO: 121 - RNA, 19 nt, Artificial Sequence
  Synthetic polynucleotide
gggcaaggccaagugggau

SEQ ID NO: 122 - RNA, 19 nt, Artificial Sequence
  Synthetic polynucleotide
ggcaaggccaagugggaug

SEQ ID NO: 123 - RNA, 19 nt, Artificial Sequence
  Synthetic polynucleotide
gcaaggccaagugggaugc

SEQ ID NO: 124 - RNA, 19 nt, Artificial Sequence
  Synthetic polynucleotide
caaggccaagugggaugcc

SEQ ID NO: 125 - RNA, 19 nt, Artificial Sequence
  Synthetic polynucleotide
aaggccaagugggaugccu

SEQ ID NO: 126 - RNA, 19 nt, Artificial Sequence
  Synthetic polynucleotide
aggccaagugggaugccug

SEQ ID NO: 127 - RNA, 19 nt, Artificial Sequence
  Synthetic polynucleotide
ggccaagugggaugccugg

SEQ ID NO: 128 - RNA, 19 nt, Artificial Sequence
  Synthetic polynucleotide
gccaagugggaugccugga

SEQ ID NO: 129 - RNA, 19 nt, Artificial Sequence
  Synthetic polynucleotide
ccaagugggaugccuggaa

SEQ ID NO: 130 - RNA, 19 nt, Artificial Sequence
  Synthetic polynucleotide
caagugggaugccuggaau

SEQ ID NO: 131 - RNA, 19 nt, Artificial Sequence
  Synthetic polynucleotide
aagugggaugccuggaaug

SEQ ID NO: 132 - RNA, 19 nt, Artificial Sequence
  Synthetic polynucleotide
agugggaugccuggaauga

SEQ ID NO: 133 - RNA, 19 nt, Artificial Sequence
  Synthetic polynucleotide
gugggaugccuggaaugag

SEQ ID NO: 134 - RNA, 19 nt, Artificial Sequence
  Synthetic polynucleotide
ugggaugccuggaaugagc

SEQ ID NO: 135 - RNA, 19 nt, Artificial Sequence
  Synthetic polynucleotide
gggaugccuggaaugagcu

SEQ ID NO: 136 - RNA, 19 nt, Artificial Sequence
  Synthetic polynucleotide
ggaugccuggaaugagcug

SEQ ID NO: 137 - RNA, 19 nt, Artificial Sequence
  Synthetic polynucleotide
gaugccuggaaugagcuga

SEQ ID NO: 138 - RNA, 19 nt, Artificial Sequence
  Synthetic polynucleotide
augccuggaaugagcugaa

SEQ ID NO: 139 - RNA, 19 nt, Artificial Sequence
  Synthetic polynucleotide
ugccuggaaugagcugaaa

SEQ ID NO: 140 - RNA, 19 nt, Artificial Sequence
  Synthetic polynucleotide
gccuggaaugagcugaaag

SEQ ID NO: 141 - RNA, 19 nt, Artificial Sequence
  Synthetic polynucleotide
ccuggaaugagcugaaagg

SEQ ID NO: 142 - RNA, 19 nt, Artificial Sequence
  Synthetic polynucleotide
cuggaaugagcugaaaggg

SEQ ID NO: 143 - RNA, 19 nt, Artificial Sequence
  Synthetic polynucleotide
uggaaugagcugaaaggga

SEQ ID NO: 144 - RNA, 19 nt, Artificial Sequence
  Synthetic polynucleotide
ggaaugagcugaaagggac

SEQ ID NO: 145 - RNA, 19 nt, Artificial Sequence
  Synthetic polynucleotide
gaaugagcugaaagggacu

SEQ ID NO: 146 - RNA, 19 nt, Artificial Sequence
  Synthetic polynucleotide
aaugagcugaaagggacuu

SEQ ID NO: 147 - RNA, 19 nt, Artificial Sequence
  Synthetic polynucleotide
augagcugaaagggacuuc

SEQ ID NO: 148 - RNA, 19 nt, Artificial Sequence
  Synthetic polynucleotide
ugagcugaaagggacuucc

SEQ ID NO: 149 - RNA, 19 nt, Artificial Sequence
  Synthetic polynucleotide
gagcugaaagggacuucca

SEQ ID NO: 150 - RNA, 19 nt, Artificial Sequence
  Synthetic polynucleotide
agcugaaagggacuuccaa

SEQ ID NO: 151 - RNA, 19 nt, Artificial Sequence
  Synthetic polynucleotide
gcugaaagggacuuccaag

SEQ ID NO: 152 - RNA, 19 nt, Artificial Sequence
  Synthetic polynucleotide
cugaaagggacuuccaagg

SEQ ID NO: 153 - RNA, 19 nt, Artificial Sequence
  Synthetic polynucleotide
ugaaagggacuuccaagga

SEQ ID NO: 154 - RNA, 19 nt, Artificial Sequence
  Synthetic polynucleotide
gaaagggacuuccaaggaa

SEQ ID NO: 155 - RNA, 19 nt, Artificial Sequence
  Synthetic polynucleotide
aaagggacuuccaaggaag

SEQ ID NO: 156 - RNA, 19 nt, Artificial Sequence
  Synthetic polynucleotide
aagggacuuccaaggaaga

SEQ ID NO: 157 - RNA, 19 nt, Artificial Sequence
  Synthetic polynucleotide
agggacuuccaaggaagau

SEQ ID NO: 158 - RNA, 19 nt, Artificial Sequence
  Synthetic polynucleotide
gggacuuccaaggaagaug

SEQ ID NO: 159 - RNA, 19 nt, Artificial Sequence
  Synthetic polynucleotide
ggacuuccaaggaagaugc

SEQ ID NO: 160 - RNA, 19 nt, Artificial Sequence
  Synthetic polynucleotide
gacuuccaaggaagaugcc

SEQ ID NO: 161 - RNA, 19 nt, Artificial Sequence
  Synthetic polynucleotide
acuuccaaggaagaugcca

SEQ ID NO: 162 - RNA, 19 nt, Artificial Sequence
  Synthetic polynucleotide
cuuccaaggaagaugccau

SEQ ID NO: 163 - RNA, 19 nt, Artificial Sequence
  Synthetic polynucleotide
uuccaaggaagaugccaug

SEQ ID NO: 164 - RNA, 19 nt, Artificial Sequence
  Synthetic polynucleotide
uccaaggaagaugccauga

SEQ ID NO: 165 - RNA, 19 nt, Artificial Sequence
  Synthetic polynucleotide
ccaaggaagaugccaugaa

SEQ ID NO: 166 - RNA, 19 nt, Artificial Sequence
  Synthetic polynucleotide
caaggaagaugccaugaaa

SEQ ID NO: 167 - RNA, 19 nt, Artificial Sequence
  Synthetic polynucleotide
aaggaagaugccaugaaag

SEQ ID NO: 168 - RNA, 19 nt, Artificial Sequence
  Synthetic polynucleotide
aggaagaugccaugaaagc

SEQ ID NO: 169 - RNA, 19 nt, Artificial Sequence
  Synthetic polynucleotide
ggaagaugccaugaaagcu

SEQ ID NO: 170 - RNA, 19 nt, Artificial Sequence
  Synthetic polynucleotide
gaagaugccaugaaagcuu

SEQ ID NO: 171 - RNA, 19 nt, Artificial Sequence
  Synthetic polynucleotide
aagaugccaugaaagcuua

SEQ ID NO: 172 - RNA, 19 nt, Artificial Sequence
  Synthetic polynucleotide
agaugccaugaaagcuuac

SEQ ID NO: 173 - RNA, 19 nt, Artificial Sequence
  Synthetic polynucleotide
gaugccaugaaagcuuaca

SEQ ID NO: 174 - RNA, 19 nt, Artificial Sequence
  Synthetic polynucleotide
augccaugaaagcuuacau

SEQ ID NO: 175 - RNA, 19 nt, Artificial Sequence
  Synthetic polynucleotide
ugccaugaaagcuuacauc

SEQ ID NO: 176 - RNA, 19 nt, Artificial Sequence
  Synthetic polynucleotide
gccaugaaagcuuacauca

SEQ ID NO: 177 - RNA, 19 nt, Artificial Sequence
  Synthetic polynucleotide
ccaugaaagcuuacaucaa

SEQ ID NO: 178 - RNA, 19 nt, Artificial Sequence
  Synthetic polynucleotide
caugaaagcuuacaucaac

SEQ ID NO: 179 - RNA, 19 nt, Artificial Sequence
  Synthetic polynucleotide
augaaagcuuacaucaaca

SEQ ID NO: 180 - RNA, 19 nt, Artificial Sequence
  Synthetic polynucleotide
ugaaagcuuacaucaacaa

SEQ ID NO: 181 - RNA, 19 nt, Artificial Sequence
  Synthetic polynucleotide
gaaagcuuacaucaacaaa

SEQ ID NO: 182 - RNA, 19 nt, Artificial Sequence
  Synthetic polynucleotide
aaagcuuacaucaacaaag

SEQ ID NO: 183 - RNA, 19 nt, Artificial Sequence
  Synthetic polynucleotide
aagcuuacaucaacaaagu

SEQ ID NO: 184 - RNA, 19 nt, Artificial Sequence
  Synthetic polynucleotide
agcuuacaucaacaaagua

SEQ ID NO: 185 - RNA, 19 nt, Artificial Sequence
  Synthetic polynucleotide
gcuuacaucaacaaaguag

SEQ ID NO: 186 - RNA, 19 nt, Artificial Sequence
  Synthetic polynucleotide
cuuacaucaacaaaguaga

SEQ ID NO: 187 - RNA, 19 nt, Artificial Sequence
  Synthetic polynucleotide
uuacaucaacaaaguagaa

SEQ ID NO: 188 - RNA, 19 nt, Artificial Sequence
  Synthetic polynucleotide
uacaucaacaaaguagaag

SEQ ID NO: 189 - RNA, 19 nt, Artificial Sequence
  Synthetic polynucleotide
acaucaacaaaguagaaga

SEQ ID NO: 190 - RNA, 19 nt, Artificial Sequence
  Synthetic polynucleotide
caucaacaaaguagaagag

SEQ ID NO: 191 - RNA, 19 nt, Artificial Sequence
  Synthetic polynucleotide
aucaacaaaguagaagagc

SEQ ID NO: 192 - RNA, 19 nt, Artificial Sequence
  Synthetic polynucleotide
ucaacaaaguagaagagcu

SEQ ID NO: 193 - RNA, 19 nt, Artificial Sequence
  Synthetic polynucleotide
caacaaaguagaagagcua

SEQ ID NO: 194 - RNA, 19 nt, Artificial Sequence
  Synthetic polynucleotide
aacaaaguagaagagcuaa

SEQ ID NO: 195 - RNA, 19 nt, Artificial Sequence
  Synthetic polynucleotide
acaaaguagaagagcuaaa

SEQ ID NO: 196 - RNA, 19 nt, Artificial Sequence
  Synthetic polynucleotide
caaaguagaagagcuaaag

SEQ ID NO: 197 - RNA, 19 nt, Artificial Sequence
  Synthetic polynucleotide
aaaguagaagagcuaaaga

SEQ ID NO: 198 - RNA, 19 nt, Artificial Sequence
  Synthetic polynucleotide
aaguagaagagcuaaagaa

SEQ ID NO: 199 - RNA, 19 nt, Artificial Sequence
  Synthetic polynucleotide
aguagaagagcuaaagaaa

SEQ ID NO: 200 - RNA, 19 nt, Artificial Sequence
  Synthetic polynucleotide
guagaagagcuaaagaaaa

SEQ ID NO: 201 - RNA, 19 nt, Artificial Sequence
  Synthetic polynucleotide
uagaagagcuaaagaaaaa

SEQ ID NO: 202 - RNA, 19 nt, Artificial Sequence
  Synthetic polynucleotide
agaagagcuaaagaaaaaa

SEQ ID NO: 203 - RNA, 19 nt, Artificial Sequence
  Synthetic polynucleotide
gaagagcuaaagaaaaaau

SEQ ID NO: 204 - RNA, 19 nt, Artificial Sequence
  Synthetic polynucleotide
aagagcuaaagaaaaaaua

SEQ ID NO: 205 - RNA, 19 nt, Artificial Sequence
  Synthetic polynucleotide
agagcuaaagaaaaaauac

SEQ ID NO: 206 - RNA, 19 nt, Artificial Sequence
  Synthetic polynucleotide
gagcuaaagaaaaaauacg

SEQ ID NO: 207 - RNA, 19 nt, Artificial Sequence
  Synthetic polynucleotide
agcuaaagaaaaaauacgg

SEQ ID NO: 208 - RNA, 19 nt, Artificial Sequence
  Synthetic polynucleotide
gcuaaagaaaaaauacggg

SEQ ID NO: 209 - RNA, 19 nt, Artificial Sequence
  Synthetic polynucleotide
gaaacgagauuccuucauc

SEQ ID NO: 210 - RNA, 19 nt, Artificial Sequence
  Synthetic polynucleotide
caagaucgguuuuggcaua

SEQ ID NO: 211 - RNA, 19 nt, Artificial Sequence
  Synthetic polynucleotide
caaacaaucugugacuauu

SEQ ID NO: 212 - RNA, 19 nt, Artificial Sequence
  Synthetic polynucleotide
caacuuguuugacgacgaa

SEQ ID NO: 213 - RNA, 19 nt, Artificial Sequence
  Synthetic polynucleotide
gaaaggauuuggcuacaaa

SEQ ID NO: 214 - RNA, 19 nt, Artificial Sequence
  Synthetic polynucleotide
gagcauguuuacaacguuu

SEQ ID NO: 215 - RNA, 19 nt, Artificial Sequence
  Synthetic polynucleotide
ggaaagacuguuccaaaaa

SEQ ID NO: 216 - RNA, 19 nt, Artificial Sequence
  Synthetic polynucleotide
acauuuacauucucuugga

SEQ ID NO: 217 - RNA, 19 nt, Artificial Sequence
  Synthetic polynucleotide
gaaagagcaucuacgguga

SEQ ID NO: 218 - RNA, 19 nt, Artificial Sequence
  Synthetic polynucleotide
acaaggagauuuauaccua

SEQ ID NO: 219 - RNA, 19 nt, Artificial Sequence
  Synthetic polynucleotide
aggaacauuuggagaguua

SEQ ID NO: 220 - RNA, 19 nt, Artificial Sequence
  Synthetic polynucleotide
caucguuuauagagacuua

SEQ ID NO: 221 - RNA, 19 nt, Artificial Sequence
  Synthetic polynucleotide
gaaaguaagcagaaucuau

SEQ ID NO: 222 - RNA, 19 nt, Artificial Sequence
  Synthetic polynucleotide
aaccaacgguuuacgucua

SEQ ID NO: 223 - RNA, 19 nt, Artificial Sequence
  Synthetic polynucleotide
gaaagacaagagaccaaua

SEQ ID NO: 224 - RNA, 19 nt, Artificial Sequence
  Synthetic polynucleotide
caacagcgauuuuaggaua

SEQ ID NO: 225 - RNA, 19 nt, Artificial Sequence
  Synthetic polynucleotide
ggaaacagcugcucauuca

SEQ ID NO: 226 - RNA, 19 nt, Artificial Sequence
  Synthetic polynucleotide
acauuuggcugugauggua

SEQ ID NO: 227 - RNA, 19 nt, Artificial Sequence
  Synthetic polynucleotide
gaaacuacaacuuacauga

SEQ ID NO: 228 - RNA, 19 nt, Artificial Sequence
  Synthetic polynucleotide
gaugauguuuuccuacuuu

SEQ ID NO: 229 - RNA, 19 nt, Artificial Sequence
  Synthetic polynucleotide
agaauuugcgauguacuua

SEQ ID NO: 230 - RNA, 19 nt, Artificial Sequence
  Synthetic polynucleotide
agguuugcuugauguuaca

SEQ ID NO: 231 - RNA, 19 nt, Artificial Sequence
  Synthetic polynucleotide
cgaaaggucuuaccggaaa

SEQ ID NO: 232 - RNA, 19 nt, Artificial Sequence
  Synthetic polynucleotide
caaagaguaugucgaauca

SEQ ID NO: 233 - RNA, 19 nt, Artificial Sequence
  Synthetic polynucleotide
aaggaaagcugacugauaa

SEQ ID NO: 234 - RNA, 19 nt, Artificial Sequence
  Synthetic polynucleotide
caucaaagcugccauggaa

SEQ ID NO: 235 - RNA, 19 nt, Artificial Sequence
  Synthetic polynucleotide
cagcauuuaacgucauaug

SEQ ID NO: 236 - RNA, 19 nt, Artificial Sequence
  Synthetic polynucleotide
auugcguuuacacuuacag

SEQ ID NO: 237 - RNA, 19 nt, Artificial Sequence
  Synthetic polynucleotide
gaucaaagguuuucacuaa

SEQ ID NO: 238 - RNA, 19 nt, Artificial Sequence
  Synthetic polynucleotide
caaaccagcgcgcuaauga

SEQ ID NO: 239 - RNA, 19 nt, Artificial Sequence
  Synthetic polynucleotide
caaacagcgucuccuggaa

SEQ ID NO: 240 - RNA, 19 nt, Artificial Sequence
  Synthetic polynucleotide
gaaagcauauacagcaauu

SEQ ID NO: 241 - RNA, 19 nt, Artificial Sequence
  Synthetic polynucleotide
caaagaagauaaccaaugu

SEQ ID NO: 242 - RNA, 19 nt, Artificial Sequence
  Synthetic polynucleotide
gaaacauccuccggcugaa

SEQ ID NO: 243 - RNA, 19 nt, Artificial Sequence
  Synthetic polynucleotide
gagcagauuuuaagaguaa

SEQ ID NO: 244 - RNA, 19 nt, Artificial Sequence
  Synthetic polynucleotide
ggacaauuuugcagcaaca

SEQ ID NO: 245 - RNA, 19 nt, Artificial Sequence
  Synthetic polynucleotide
caaaggugcugcugaugua

SEQ ID NO: 246 - RNA, 19 nt, Artificial Sequence
  Synthetic polynucleotide
acaucauuuacggcaugua

SEQ ID NO: 247 - RNA, 19 nt, Artificial Sequence
  Synthetic polynucleotide
ccacaccgcuggccaagaa

SEQ ID NO: 248 - RNA, 19 nt, Artificial Sequence
  Synthetic polynucleotide
uaucaagccugccaauaug

SEQ ID NO: 249 - RNA, 19 nt, Artificial Sequence
  Synthetic polynucleotide
ucaauaagccaucuucuaa

SEQ ID NO: 250 - RNA, 19 nt, Artificial Sequence
  Synthetic polynucleotide
gagcuaaucugccacauug

SEQ ID NO: 251 - RNA, 19 nt, Artificial Sequence
  Synthetic polynucleotide
cgaaggcgguggccaauca

SEQ ID NO: 252 - RNA, 19 nt, Artificial Sequence
  Synthetic polynucleotide
gcacauccguuggccauca

SEQ ID NO: 253 - RNA, 19 nt, Artificial Sequence
  Synthetic polynucleotide
gccaggcaaugaacaggaa

SEQ ID NO: 254 - RNA, 19 nt, Artificial Sequence
  Synthetic polynucleotide
ggaagccagcgcucaauug

SEQ ID NO: 255 - RNA, 19 nt, Artificial Sequence
  Synthetic polynucleotide
gcgagccacuggugcugaa

SEQ ID NO: 256 - RNA, 19 nt, Artificial Sequence
  Synthetic polynucleotide
ccaccuccguugccauaug

SEQ ID NO: 257 - RNA, 19 nt, Artificial Sequence
  Synthetic polynucleotide
gaaggacucuagccaguua

SEQ ID NO: 258 - RNA, 19 nt, Artificial Sequence
  Synthetic polynucleotide
gaccacaccuugccaucaa

SEQ ID NO: 259 - RNA, 19 nt, Artificial Sequence
  Synthetic polynucleotide
gcaguuaccagugccaaua

SEQ ID NO: 260 - RNA, 19 nt, Artificial Sequence
  Synthetic polynucleotide
ggacgccaaugaccuagaa

SEQ ID NO: 261 - RNA, 19 nt, Artificial Sequence
  Synthetic polynucleotide
gaacuaugcugccaugaag

SEQ ID NO: 262 - RNA, 19 nt, Artificial Sequence
  Synthetic polynucleotide
caucaaagcugccauggaa

SEQ ID NO: 263 - RNA, 19 nt, Artificial Sequence
  Synthetic polynucleotide
agacauugcugccacagua

SEQ ID NO: 264 - RNA, 19 nt, Artificial Sequence
  Synthetic polynucleotide
ggccaguucuacgucauga

SEQ ID NO: 265 - RNA, 19 nt, Artificial Sequence
  Synthetic polynucleotide
gccaggagcugaacauuaa

SEQ ID NO: 266 - RNA, 19 nt, Artificial Sequence
  Synthetic polynucleotide
cacguccgcucaagagugu

SEQ ID NO: 267 - RNA, 19 nt, Artificial Sequence
  Synthetic polynucleotide
uaacaaugcugggaauuca

SEQ ID NO: 268 - RNA, 19 nt, Artificial Sequence
  Synthetic polynucleotide
agacagagcuugagaauaa

SEQ ID NO: 269 - RNA, 19 nt, Artificial Sequence
  Synthetic polynucleotide
uguugcagcuuccuacuuc

SEQ ID NO: 270 - RNA, 19 nt, Artificial Sequence
  Synthetic polynucleotide
gaccaaggcuuacugaaua

SEQ ID NO: 271 - RNA, 19 nt, Artificial Sequence
  Synthetic polynucleotide
cauaguuggugaagacaua

SEQ ID NO: 272 - RNA, 19 nt, Artificial Sequence
  Synthetic polynucleotide
gagcuccacuucaaugaga

SEQ ID NO: 273 - RNA, 19 nt, Artificial Sequence
  Synthetic polynucleotide
gaacagaaguuacguuguc

SEQ ID NO: 274 - RNA, 19 nt, Artificial Sequence
  Synthetic polynucleotide
caccuggacuccuaugaga

SEQ ID NO: 275 - RNA, 19 nt, Artificial Sequence
  Synthetic polynucleotide
cuacaacuguccuaaccca

SEQ ID NO: 276 - RNA, 19 nt, Artificial Sequence
  Synthetic polynucleotide
cuacacaauugucacauua

SEQ ID NO: 277 - RNA, 19 nt, Artificial Sequence
  Synthetic polynucleotide
ugaggaggcugaguaauuc

SEQ ID NO: 278 - RNA, 19 nt, Artificial Sequence
  Synthetic polynucleotide
ccacagaacucaugaacau

SEQ ID NO: 279 - RNA, 19 nt, Artificial Sequence
  Synthetic polynucleotide
ucaacuucaucacugagua

SEQ ID NO: 280 - RNA, 19 nt, Artificial Sequence
  Synthetic polynucleotide
cgucagagcuucuccguaa

SEQ ID NO: 281 - RNA, 19 nt, Artificial Sequence
  Synthetic polynucleotide
cguagccgcugccuaugua

SEQ ID NO: 282 - RNA, 19 nt, Artificial Sequence
  Synthetic polynucleotide
acagcgacguguucucuga

SEQ ID NO: 283 - RNA, 19 nt, Artificial Sequence
  Synthetic polynucleotide
cagaaggccucgaaucuca

SEQ ID NO: 284 - RNA, 19 nt, Artificial Sequence
  Synthetic polynucleotide
gcucaaggcuggugaugug

SEQ ID NO: 285 - RNA, 19 nt, Artificial Sequence
  Synthetic polynucleotide
gaugacauacagccuauca

SEQ ID NO: 286 - RNA, 19 nt, Artificial Sequence
  Synthetic polynucleotide
uuuauucgauuccucguua

SEQ ID NO: 287 - RNA, 19 nt, Artificial Sequence
  Synthetic polynucleotide
ucgaguggaugauguaaua

SEQ ID NO: 288 - RNA, 19 nt, Artificial Sequence
  Synthetic polynucleotide
aggaucugcauagagucuu

SEQ ID NO: 289 - RNA, 19 nt, Artificial Sequence
  Synthetic polynucleotide
caaccuggauggugacaug

SEQ ID NO: 290 - RNA, 19 nt, Artificial Sequence
  Synthetic polynucleotide
ugaagaacgguaccagaug

SEQ ID NO: 291 - RNA, 19 nt, Artificial Sequence
  Synthetic polynucleotide
gugagacagugacggauua

SEQ ID NO: 292 - RNA, 19 nt, Artificial Sequence
  Synthetic polynucleotide
acgaacaggugaugcacua

SEQ ID NO: 293 - RNA, 19 nt, Artificial Sequence
  Synthetic polynucleotide
aauaaugaauggacgacuu

SEQ ID NO: 294 - RNA, 19 nt, Artificial Sequence
  Synthetic polynucleotide
gaugaccuguccauagaug

SEQ ID NO: 295 - RNA, 19 nt, Artificial Sequence
  Synthetic polynucleotide
ucuaugaccuguccaagga

SEQ ID NO: 296 - RNA, 19 nt, Artificial Sequence
  Synthetic polynucleotide
auacagaucuggucgaguu

SEQ ID NO: 297 - RNA, 19 nt, Artificial Sequence
  Synthetic polynucleotide
cauauugacuuccuguauu

SEQ ID NO: 298 - RNA, 19 nt, Artificial Sequence
  Synthetic polynucleotide
aaguugucauggaugaccu

SEQ ID NO: 299 - RNA, 19 nt, Artificial Sequence
  Synthetic polynucleotide
agugaguacacaaaugucc

SEQ ID NO: 300 - RNA, 19 nt, Artificial Sequence
  Synthetic polynucleotide
uucuuggcacucgguguuc

SEQ ID NO: 301 - RNA, 19 nt, Artificial Sequence
  Synthetic polynucleotide
auacucuucaaauuuccgg

SEQ ID NO: 302 - RNA, 19 nt, Artificial Sequence
  Synthetic polynucleotide
uacaugccguaaaugaugu

SEQ ID NO: 303 - RNA, 19 nt, Artificial Sequence
  Synthetic polynucleotide
agaugccaugaaagcuuac

SEQ ID NO: 304 - RNA, 19 nt, Artificial Sequence
  Synthetic polynucleotide
uacaucagcagcaccuuug

SEQ ID NO: 305 - RNA, 19 nt, Artificial Sequence
  Synthetic polynucleotide
uuaauguucagcuccuggc

SEQ ID NO: 306 - RNA, 19 nt, Artificial Sequence
  Synthetic polynucleotide
uuccaaggaagaugccaug

SEQ ID NO: 307 - RNA, 19 nt, Artificial Sequence
  Synthetic polynucleotide
uuauugacaaacuccugga

SEQ ID NO: 308 - RNA, 19 nt, Artificial Sequence
  Synthetic polynucleotide
uguugcugcaaaauugucc

SEQ ID NO: 309 - RNA, 19 nt, Artificial Sequence
  Synthetic polynucleotide
agagacuguagguaguuuc

SEQ ID NO: 310 - RNA, 19 nt, Artificial Sequence
  Synthetic polynucleotide
uucagccggaggauguuuc

SEQ ID NO: 311 - RNA, 19 nt, Artificial Sequence
  Synthetic polynucleotide
acauugguuaucuucuuug

SEQ ID NO: 312 - RNA, 19 nt, Artificial Sequence
  Synthetic polynucleotide
augaccuuggccaggggug

SEQ ID NO: 313 - RNA, 19 nt, Artificial Sequence
  Synthetic polynucleotide
uuacucuuaaaaucugcuc

SEQ ID NO: 314 - RNA, 19 nt, Artificial Sequence
  Synthetic polynucleotide
uuauugacaaacuccugga

SEQ ID NO: 315 - RNA, 19 nt, Artificial Sequence
  Synthetic polynucleotide
aaaaucagagagauccuca

SEQ ID NO: 316 - RNA, 19 nt, Artificial Sequence
  Synthetic polynucleotide
aauugcuguauaugcuuuc

SEQ ID NO: 317 - RNA, 19 nt, Artificial Sequence
  Synthetic polynucleotide
ucaugacguagaacuggcc

SEQ ID NO: 318 - RNA, 19 nt, Artificial Sequence
  Synthetic polynucleotide
uuccaggagacgcuguuug

SEQ ID NO: 319 - RNA, 19 nt, Artificial Sequence
  Synthetic polynucleotide
auggaugaccuuggccagg

SEQ ID NO: 320 - RNA, 19 nt, Artificial Sequence
  Synthetic polynucleotide
caauggcucaaguaucugc

SEQ ID NO: 321 - RNA, 19 nt, Artificial Sequence
  Synthetic polynucleotide
uguuuguggacgaaguacc

SEQ ID NO: 322 - RNA, 19 nt, Artificial Sequence
  Synthetic polynucleotide
uucgagggagaaguucuuc

SEQ ID NO: 323 - RNA, 19 nt, Artificial Sequence
  Synthetic polynucleotide
uuccaggagacgcuguuug

SEQ ID NO: 324 - RNA, 19 nt, Artificial Sequence
  Synthetic polynucleotide
uuagugaaaaccuuugauc

SEQ ID NO: 325 - RNA, 19 nt, Artificial Sequence
  Synthetic polynucleotide
uacuguggcagcaaugucu

SEQ ID NO: 326 - RNA, 19 nt, Artificial Sequence
  Synthetic polynucleotide
ugcuccaaaacaacaacgg

SEQ ID NO: 327 - RNA, 19 nt, Artificial Sequence
  Synthetic polynucleotide
caugugagcagguccuccg

SEQ ID NO: 328 - RNA, 19 nt, Artificial Sequence
  Synthetic polynucleotide
augagcugaaagggacuuc

SEQ ID NO: 329 - RNA, 19 nt, Artificial Sequence
  Synthetic polynucleotide
uauugguugucacugauca

SEQ ID NO: 330 - RNA, 19 nt, Artificial Sequence
  Synthetic polynucleotide
uucuugcgucgaguuuucc

SEQ ID NO: 331 - RNA, 19 nt, Artificial Sequence
  Synthetic polynucleotide
uuccaagaccugccuacca

SEQ ID NO: 332 - RNA, 19 nt, Artificial Sequence
  Synthetic polynucleotide
uugcgcggaggaguugugu

SEQ ID NO: 333 - RNA, 19 nt, Artificial Sequence
  Synthetic polynucleotide
ugucauggaugaccuuggc

SEQ ID NO: 334 - RNA, 19 nt, Artificial Sequence
  Synthetic polynucleotide
cugcuuagcaccccuggcc

SEQ ID NO: 335 - RNA, 19 nt, Artificial Sequence
  Synthetic polynucleotide
agucccuuucagcucauuc

SEQ ID NO: 336 - RNA, 19 nt, Artificial Sequence
  Synthetic polynucleotide
auguuuacuaccaaaugga

SEQ ID NO: 337 - RNA, 19 nt, Artificial Sequence
  Synthetic polynucleotide
uuuggagcacggaaagacg

SEQ ID NO: 338 - RNA, 19 nt, Artificial Sequence
  Synthetic polynucleotide
uuguuacuugacuggcgac

SEQ ID NO: 339 - RNA, 19 nt, Artificial Sequence
  Synthetic polynucleotide
aacuucccgccgccguugu

SEQ ID NO: 340 - RNA, 19 nt, Artificial Sequence
  Synthetic polynucleotide
auggcaucuuccuuggaag

SEQ ID NO: 341 - RNA, 19 nt, Artificial Sequence
  Synthetic polynucleotide
uuccauggcagcuuugaug

SEQ ID NO: 342 - RNA, 19 nt, Artificial Sequence
  Synthetic polynucleotide
uuccauggcagcuuugaug

SEQ ID NO: 343 - RNA, 19 nt, Artificial Sequence
  Synthetic polynucleotide
ucuuuccgugcuccaaaac

SEQ ID NO: 344 - RNA, 19 nt, Artificial Sequence
  Synthetic polynucleotide
aguugcuucaaaucugcuc

SEQ ID NO: 345 - RNA, 19 nt, Artificial Sequence
  Synthetic polynucleotide
agcaccccuggccaagguc

SEQ ID NO: 346 - RNA, 19 nt, Artificial Sequence
  Synthetic polynucleotide
auugcguuuacacuuacag

SEQ ID NO: 347 - RNA, 19 nt, Artificial Sequence
  Synthetic polynucleotide
auguucaugaguucugugg

SEQ ID NO: 348 - RNA, 19 nt, Artificial Sequence
  Synthetic polynucleotide
ugauucgacauacucuuug

SEQ ID NO: 349 - RNA, 19 nt, Artificial Sequence
  Synthetic polynucleotide
ucauuagcgcgcugguuug

SEQ ID NO: 350 - RNA, 19 nt, Artificial Sequence
  Synthetic polynucleotide
uucauggcaucuuccuugg

SEQ ID NO: 351 - RNA, 19 nt, Artificial Sequence
  Synthetic polynucleotide
ucgaguuuuccgguaagac

SEQ ID NO: 352 - RNA, 19 nt, Artificial Sequence
  Synthetic polynucleotide
ucaucgucuuuccgugcuc

SEQ ID NO: 353 - RNA, 19 nt, Artificial Sequence
  Synthetic polynucleotide
ugauuuuucuugcgucgag

SEQ ID NO: 354 - RNA, 19 nt, Artificial Sequence
  Synthetic polynucleotide
aggucuuaccggaaaacuc

SEQ ID NO: 355 - RNA, 19 nt, Artificial Sequence
  Synthetic polynucleotide
aaggaagaugccaugaaag

SEQ ID NO: 356 - RNA, 19 nt, Artificial Sequence
  Synthetic polynucleotide
auaggcagcagcgaguucc

SEQ ID NO: 357 - RNA, 19 nt, Artificial Sequence
  Synthetic polynucleotide
aacaguggcauaggcuuuc

SEQ ID NO: 358 - RNA, 19 nt, Artificial Sequence
  Synthetic polynucleotide
aucgucuuuccgugcucca

SEQ ID NO: 359 - RNA, 19 nt, Artificial Sequence
  Synthetic polynucleotide
agagauccucauaaaggcc

SEQ ID NO: 360 - RNA, 19 nt, Artificial Sequence
  Synthetic polynucleotide
ucucucugauuuuucuugc

SEQ ID NO: 361 - RNA, 19 nt, Artificial Sequence
  Synthetic polynucleotide
uacuucguccacaaacaca

SEQ ID NO: 362 - RNA, 19 nt, Artificial Sequence
  Synthetic polynucleotide
ugagcugaaagggacuucc

SEQ ID NO: 363 - RNA, 19 nt, Artificial Sequence
  Synthetic polynucleotide
uuggccuuuaugaggaucu

SEQ ID NO: 364 - RNA, 19 nt, Artificial Sequence
  Synthetic polynucleotide
agagaucguggauuacguc

SEQ ID NO: 365 - RNA, 19 nt, Artificial Sequence
  Synthetic polynucleotide
aacggcggcgggaaguuca

SEQ ID NO: 366 - RNA, 19 nt, Artificial Sequence
  Synthetic polynucleotide
aacugcuuagcaccccugg

SEQ ID NO: 367 - RNA, 19 nt, Artificial Sequence
  Synthetic polynucleotide
uauccuucaugcccauucc

SEQ ID NO: 368 - RNA, 19 nt, Artificial Sequence
  Synthetic polynucleotide
agcuuucauggcaucuucc

SEQ ID NO: 369 - RNA, 19 nt, Artificial Sequence
  Synthetic polynucleotide
uucuaggucauuggcgucc

SEQ ID NO: 370 - RNA, 19 nt, Artificial Sequence
  Synthetic polynucleotide
aaaguaggaaaacaucauc

SEQ ID NO: 371 - RNA, 19 nt, Artificial Sequence
  Synthetic polynucleotide
uuagcaccccuggccaagg

SEQ ID NO: 372 - RNA, 19 nt, Artificial Sequence
  Synthetic polynucleotide
uauuggcacugguaacugc

SEQ ID NO: 373 - RNA, 19 nt, Artificial Sequence
  Synthetic polynucleotide
aggaagaugccaugaaagc

SEQ ID NO: 374 - RNA, 19 nt, Artificial Sequence
  Synthetic polynucleotide
uuuccgguaagaccuuucg

SEQ ID NO: 375 - RNA, 19 nt, Artificial Sequence
  Synthetic polynucleotide
uuuccgguaagaccuuucg

SEQ ID NO: 376 - RNA, 19 nt, Artificial Sequence
  Synthetic polynucleotide
ucauguaaguuguaguuuc

SEQ ID NO: 377 - RNA, 19 nt, Artificial Sequence
  Synthetic polynucleotide
uggacgaaguaccgaaagg

SEQ ID NO: 378 - RNA, 19 nt, Artificial Sequence
  Synthetic polynucleotide
uuauucucaagcucugucu

SEQ ID NO: 379 - RNA, 19 nt, Artificial Sequence
  Synthetic polynucleotide
caaguggccagggcauagc

SEQ ID NO: 380 - RNA, 19 nt, Artificial Sequence
  Synthetic polynucleotide
augaggaugauggguauga

SEQ ID NO: 381 - RNA, 19 nt, Artificial Sequence
  Synthetic polynucleotide
uaguugaccagcucauccg

SEQ ID NO: 382 - RNA, 19 nt, Artificial Sequence
  Synthetic polynucleotide
uugauggcaaggugugguc

SEQ ID NO: 383 - RNA, 19 nt, Artificial Sequence
  Synthetic polynucleotide
uaaguacaucgcaaauucu

SEQ ID NO: 384 - RNA, 19 nt, Artificial Sequence
  Synthetic polynucleotide
ugaaugagcagcuguuucc

SEQ ID NO: 385 - RNA, 19 nt, Artificial Sequence
  Synthetic polynucleotide
cuuaccggaaaacucgacg

SEQ ID NO: 386 - RNA, 19 nt, Artificial Sequence
  Synthetic polynucleotide
uaccaucacagccaaaugu

SEQ ID NO: 387 - RNA, 19 nt, Artificial Sequence
  Synthetic polynucleotide
acggaaaaagagaucgugg

SEQ ID NO: 388 - RNA, 19 nt, Artificial Sequence
  Synthetic polynucleotide
uaacuggcuagaguccuuc

SEQ ID NO: 389 - RNA, 19 nt, Artificial Sequence
  Synthetic polynucleotide
acuggcgacguaauccacg

SEQ ID NO: 390 - RNA, 19 nt, Artificial Sequence
  Synthetic polynucleotide
aggaucucucugauuuuuc

SEQ ID NO: 391 - RNA, 19 nt, Artificial Sequence
  Synthetic polynucleotide
cauauggcaacggaggugg

SEQ ID NO: 392 - RNA, 19 nt, Artificial Sequence
  Synthetic polynucleotide
agaucguggauuacgucgc

SEQ ID NO: 393 - RNA, 19 nt, Artificial Sequence
  Synthetic polynucleotide
aaguaccgaaaggucuuac

SEQ ID NO: 394 - RNA, 19 nt, Artificial Sequence
  Synthetic polynucleotide
ucguccacaaacacaacuc

SEQ ID NO: 395 - RNA, 19 nt, Artificial Sequence
  Synthetic polynucleotide
uucagcaccaguggcucgc

SEQ ID NO: 396 - RNA, 19 nt, Artificial Sequence
  Synthetic polynucleotide
agagagauccucauaaagg

SEQ ID NO: 397 - RNA, 19 nt, Artificial Sequence
  Synthetic polynucleotide
caauugagcgcuggcuucc

SEQ ID NO: 398 - RNA, 19 nt, Artificial Sequence
  Synthetic polynucleotide
aaucacuccaacugaaucc

SEQ ID NO: 399 - RNA, 19 nt, Artificial Sequence
  Synthetic polynucleotide
aagacaauagucccuugga

SEQ ID NO: 400 - RNA, 19 nt, Artificial Sequence
  Synthetic polynucleotide
agaaccuccauccaugugc

SEQ ID NO: 401 - RNA, 19 nt, Artificial Sequence
  Synthetic polynucleotide
uuggccaggggugcuaagc

SEQ ID NO: 402 - RNA, 19 nt, Artificial Sequence
  Synthetic polynucleotide
cuugcgucgaguuuuccgg

SEQ ID NO: 403 - RNA, 19 nt, Artificial Sequence
  Synthetic polynucleotide
aauggcuuugaaucuuugg

SEQ ID NO: 404 - RNA, 19 nt, Artificial Sequence
  Synthetic polynucleotide
uacauaggcagcggcuacg

SEQ ID NO: 405 - RNA, 19 nt, Artificial Sequence
  Synthetic polynucleotide
agcuaaagaaaaaauacgg

SEQ ID NO: 406 - RNA, 19 nt, Artificial Sequence
  Synthetic polynucleotide
aagcuuucauggcaucuuc

SEQ ID NO: 407 - RNA, 19 nt, Artificial Sequence
  Synthetic polynucleotide
uauccuaaaaucgcuguug

SEQ ID NO: 408 - RNA, 19 nt, Artificial Sequence
  Synthetic polynucleotide
aaauucaucaucgaaguac

SEQ ID NO: 409 - RNA, 19 nt, Artificial Sequence
  Synthetic polynucleotide
uaaagguuaacauccgguc

SEQ ID NO: 410 - RNA, 19 nt, Artificial Sequence
  Synthetic polynucleotide
uauuggucucuugucuuuc

SEQ ID NO: 411 - RNA, 19 nt, Artificial Sequence
  Synthetic polynucleotide
aaugugcccguccuugucc

SEQ ID NO: 412 - RNA, 19 nt, Artificial Sequence
  Synthetic polynucleotide
uuuccgucaucgucuuucc

SEQ ID NO: 413 - RNA, 19 nt, Artificial Sequence
  Synthetic polynucleotide
guuguuguuuuggagcacg

SEQ ID NO: 414 - RNA, 19 nt, Artificial Sequence
  Synthetic polynucleotide
cacaucaccagccuugagc

SEQ ID NO: 415 - RNA, 19 nt, Artificial Sequence
  Synthetic polynucleotide
aaaggucuuaccggaaaac

SEQ ID NO: 416 - RNA, 19 nt, Artificial Sequence
  Synthetic polynucleotide
ugaggaggcugaguaauuc

SEQ ID NO: 417 - RNA, 19 nt, Artificial Sequence
  Synthetic polynucleotide
aaagagaucguggauuacg

SEQ ID NO: 418 - RNA, 19 nt, Artificial Sequence
  Synthetic polynucleotide
uuaauguccacugcgucga

SEQ ID NO: 419 - RNA, 19 nt, Artificial Sequence
  Synthetic polynucleotide
ucguacacuagcacauugc

SEQ ID NO: 420 - RNA, 19 nt, Artificial Sequence
  Synthetic polynucleotide
uuggacccguacuucauca

SEQ ID NO: 421 - RNA, 19 nt, Artificial Sequence
  Synthetic polynucleotide
uuccuguucauugccuggc

SEQ ID NO: 422 - RNA, 19 nt, Artificial Sequence
  Synthetic polynucleotide
ugauggccaacggaugugc

SEQ ID NO: 423 - RNA, 19 nt, Artificial Sequence
  Synthetic polynucleotide
aggaguuguguuuguggac

SEQ ID NO: 424 - RNA, 19 nt, Artificial Sequence
  Synthetic polynucleotide
auagugaaggcagcuguga

SEQ ID NO: 425 - RNA, 19 nt, Artificial Sequence
  Synthetic polynucleotide
guaucggaggcgugugucc

SEQ ID NO: 426 - RNA, 19 nt, Artificial Sequence
  Synthetic polynucleotide
caucugaggaggcaccugc

SEQ ID NO: 427 - RNA, 19 nt, Artificial Sequence
  Synthetic polynucleotide
uuucgcgguuguuacuuga

SEQ ID NO: 428 - RNA, 19 nt, Artificial Sequence
  Synthetic polynucleotide
aauuucuggagcccuguac

SEQ ID NO: 429 - RNA, 19 nt, Artificial Sequence
  Synthetic polynucleotide
auagauucugcuuacuuuc

SEQ ID NO: 430 - RNA, 19 nt, Artificial Sequence
  Synthetic polynucleotide
aagaccuuucgguacuucg

SEQ ID NO: 431 - RNA, 19 nt, Artificial Sequence
  Synthetic polynucleotide
cauaguaguagcccagugc

SEQ ID NO: 432 - RNA, 19 nt, Artificial Sequence
  Synthetic polynucleotide
uaguaaagcgccuccggcc

SEQ ID NO: 433 - RNA, 19 nt, Artificial Sequence
  Synthetic polynucleotide
uagucaaugcuuucuucag

SEQ ID NO: 434 - RNA, 19 nt, Artificial Sequence
  Synthetic polynucleotide
uaauccgucacugucucac

SEQ ID NO: 435 - RNA, 19 nt, Artificial Sequence
  Synthetic polynucleotide
aaaaaguugcgcggaggag

SEQ ID NO: 436 - RNA, 19 nt, Artificial Sequence
  Synthetic polynucleotide
aaacacaacuccuccgcgc

SEQ ID NO: 437 - RNA, 19 nt, Artificial Sequence
  Synthetic polynucleotide
acgucgccagucaaguaac

SEQ ID NO: 438 - RNA, 19 nt, Artificial Sequence
  Synthetic polynucleotide
uggguuaggacaguuguag

SEQ ID NO: 439 - RNA, 19 nt, Artificial Sequence
  Synthetic polynucleotide
uauuguaacacgccuaaca

SEQ ID NO: 440 - RNA, 19 nt, Artificial Sequence
  Synthetic polynucleotide
uacaaaggcaguaaggcuc

SEQ ID NO: 441 - RNA, 19 nt, Artificial Sequence
  Synthetic polynucleotide
aaaacaacaacggcggcgg

SEQ ID NO: 442 - RNA, 19 nt, Artificial Sequence
  Synthetic polynucleotide
ucucucggcagucuugucg

SEQ ID NO: 443 - RNA, 19 nt, Artificial Sequence
  Synthetic polynucleotide
auaaccggaaguccuccuc

SEQ ID NO: 444 - RNA, 19 nt, Artificial Sequence
  Synthetic polynucleotide
uccgcgcaacuuuuucgcg

SEQ ID NO: 445 - RNA, 19 nt, Artificial Sequence
  Synthetic polynucleotide
ugauaggcuguaugucauc

SEQ ID NO: 446 - RNA, 19 nt, Artificial Sequence
  Synthetic polynucleotide
aaugauacgaucgaguucc

SEQ ID NO: 447 - RNA, 19 nt, Artificial Sequence
  Synthetic polynucleotide
acagucccaucuucaucac

SEQ ID NO: 448 - RNA, 19 nt, Artificial Sequence
  Synthetic polynucleotide
aagcuuacaucaacaaagu

SEQ ID NO: 449 - RNA, 19 nt, Artificial Sequence
  Synthetic polynucleotide
ugauuggccaccgccuucg

SEQ ID NO: 450 - RNA, 19 nt, Artificial Sequence
  Synthetic polynucleotide
uaagucucuauaaacgaug

SEQ ID NO: 451 - RNA, 19 nt, Artificial Sequence
  Synthetic polynucleotide
uaccggaaaacucgacgca

SEQ ID NO: 452 - RNA, 19 nt, Artificial Sequence
  Synthetic polynucleotide
uaaugugcccguccuuguc

SEQ ID NO: 453 - RNA, 19 nt, Artificial Sequence
  Synthetic polynucleotide
uaacucuccaaauguuccu

SEQ ID NO: 454 - RNA, 19 nt, Artificial Sequence
  Synthetic polynucleotide
uuucgguacuucguccaca

SEQ ID NO: 455 - RNA, 19 nt, Artificial Sequence
  Synthetic polynucleotide
aagaagcgaugcugcauga

SEQ ID NO: 456 - RNA, 19 nt, Artificial Sequence
  Synthetic polynucleotide
accgcgaaaaaguugcgcg

SEQ ID NO: 457 - RNA, 19 nt, Artificial Sequence
  Synthetic polynucleotide
uuucauggcaucuuccuug

SEQ ID NO: 458 - RNA, 19 nt, Artificial Sequence
  Synthetic polynucleotide
uuacggagaagcucugacg

SEQ ID NO: 459 - RNA, 19 nt, Artificial Sequence
  Synthetic polynucleotide
aacaaccgcgaaaaaguug

SEQ ID NO: 460 - RNA, 19 nt, Artificial Sequence
  Synthetic polynucleotide
uuccaugcaaaugcugauc

SEQ ID NO: 461 - RNA, 19 nt, Artificial Sequence
  Synthetic polynucleotide
uacucagugaugaaguuga

SEQ ID NO: 462 - RNA, 19 nt, Artificial Sequence
  Synthetic polynucleotide
uagcucuucuacuuuguug

SEQ ID NO: 463 - RNA, 19 nt, Artificial Sequence
  Synthetic polynucleotide
uuuguggacgaaguaccga

SEQ ID NO: 464 - RNA, 19 nt, Artificial Sequence
  Synthetic polynucleotide
gagcuaaagaaaaaauacg

SEQ ID NO: 465 - RNA, 19 nt, Artificial Sequence
  Synthetic polynucleotide
uauuacaucauccacucga

SEQ ID NO: 466 - RNA, 19 nt, Artificial Sequence
  Synthetic polynucleotide
auaguugaccagcucaucc

SEQ ID NO: 467 - RNA, 19 nt, Artificial Sequence
  Synthetic polynucleotide
aagacucuaugcagauccu

SEQ ID NO: 468 - RNA, 19 nt, Artificial Sequence
  Synthetic polynucleotide
uuguugauguaagcuuuca

SEQ ID NO: 469 - RNA, 19 nt, Artificial Sequence
  Synthetic polynucleotide
aaagggacuuccaaggaag

SEQ ID NO: 470 - RNA, 19 nt, Artificial Sequence
  Synthetic polynucleotide
acuuuuucgcgguuguuac

SEQ ID NO: 471 - RNA, 19 nt, Artificial Sequence
  Synthetic polynucleotide
ucucauugaaguggagcuc

SEQ ID NO: 472 - RNA, 19 nt, Artificial Sequence
  Synthetic polynucleotide
uauucaguaagccuugguc

SEQ ID NO: 473 - RNA, 19 nt, Artificial Sequence
  Synthetic polynucleotide
aacuuguacaggucagucu

SEQ ID NO: 474 - RNA, 19 nt, Artificial Sequence
  Synthetic polynucleotide
aagaaaaaucagagagauc

SEQ ID NO: 475 - RNA, 19 nt, Artificial Sequence
  Synthetic polynucleotide
acacucuugagcggacgug

SEQ ID NO: 476 - RNA, 19 nt, Artificial Sequence
  Synthetic polynucleotide
uucuuguuaacuacugcca

SEQ ID NO: 477 - RNA, 19 nt, Artificial Sequence
  Synthetic polynucleotide
cuccaaaacaacaacggcg

SEQ ID NO: 478 - RNA, 19 nt, Artificial Sequence
  Synthetic polynucleotide
ugaacaugaaccgcccucc

SEQ ID NO: 479 - RNA, 19 nt, Artificial Sequence
  Synthetic polynucleotide
uuucuuuagcucuucuacu

SEQ ID NO: 480 - RNA, 19 nt, Artificial Sequence
  Synthetic polynucleotide
auccacgaucucuuuuucc

SEQ ID NO: 481 - RNA, 19 nt, Artificial Sequence
  Synthetic polynucleotide
auccucauaaaggccaaga

SEQ ID NO: 482 - RNA, 19 nt, Artificial Sequence
  Synthetic polynucleotide
uagguauaaaucuccuugu

SEQ ID NO: 483 - RNA, 19 nt, Artificial Sequence
  Synthetic polynucleotide
acucucgggagccuuguuc

SEQ ID NO: 484 - RNA, 19 nt, Artificial Sequence
  Synthetic polynucleotide
acaaacacaacuccuccgc

SEQ ID NO: 485 - RNA, 19 nt, Artificial Sequence
  Synthetic polynucleotide
agagcuaaagaaaaaauac

SEQ ID NO: 486 - RNA, 19 nt, Artificial Sequence
  Synthetic polynucleotide
uuacacaacacuucgaugg

SEQ ID NO: 487 - RNA, 19 nt, Artificial Sequence
  Synthetic polynucleotide
aauuccaaggugcguguug

SEQ ID NO: 488 - RNA, 19 nt, Artificial Sequence
  Synthetic polynucleotide
uacugcaacugcguguagc

SEQ ID NO: 489 - RNA, 19 nt, Artificial Sequence
  Synthetic polynucleotide
aucaucgaaguaccuugug

SEQ ID NO: 490 - RNA, 19 nt, Artificial Sequence
  Synthetic polynucleotide
uguaagcuuucauggcauc

SEQ ID NO: 491 - RNA, 19 nt, Artificial Sequence
  Synthetic polynucleotide
guaccgaaaggucuuaccg

SEQ ID NO: 492 - RNA, 19 nt, Artificial Sequence
  Synthetic polynucleotide
uuuagcucuucuacuuugu

SEQ ID NO: 493 - RNA, 19 nt, Artificial Sequence
  Synthetic polynucleotide
ucaccguagaugcucuuuc

SEQ ID NO: 494 - RNA, 19 nt, Artificial Sequence
  Synthetic polynucleotide
acuugauccagagaaccuc

SEQ ID NO: 495 - RNA, 19 nt, Artificial Sequence
  Synthetic polynucleotide
uaaugugacaauuguguag

SEQ ID NO: 496 - RNA, 19 nt, Artificial Sequence
  Synthetic polynucleotide
uuuguugauguaagcuuuc

SEQ ID NO: 497 - RNA, 19 nt, Artificial Sequence
  Synthetic polynucleotide
uuugguguaaccucccuug

SEQ ID NO: 498 - RNA, 19 nt, Artificial Sequence
  Synthetic polynucleotide
caguaaaucagauaguagc

SEQ ID NO: 499 - RNA, 19 nt, Artificial Sequence
  Synthetic polynucleotide
cuacuuuguugauguaagc

SEQ ID NO: 500 - RNA, 19 nt, Artificial Sequence
  Synthetic polynucleotide
uacugcaacaucuccgugc

SEQ ID NO: 501 - RNA, 19 nt, Artificial Sequence
  Synthetic polynucleotide
ucaauagugcaaucauuuc

SEQ ID NO: 502 - RNA, 19 nt, Artificial Sequence
  Synthetic polynucleotide
aacaacaacggcggcggga

SEQ ID NO: 503 - RNA, 19 nt, Artificial Sequence
  Synthetic polynucleotide
uacuucaucacuaucuccc

SEQ ID NO: 504 - RNA, 19 nt, Artificial Sequence
  Synthetic polynucleotide
acgggcaaggccaaguggg

SEQ ID NO: 505 - RNA, 19 nt, Artificial Sequence
  Synthetic polynucleotide
ucuaguagcagcgucuccc

SEQ ID NO: 506 - RNA, 19 nt, Artificial Sequence
  Synthetic polynucleotide
aaguccuccagguaguugg

SEQ ID NO: 507 - RNA, 19 nt, Artificial Sequence
  Synthetic polynucleotide
uucucagggaccucaauag

SEQ ID NO: 508 - RNA, 19 nt, Artificial Sequence
  Synthetic polynucleotide
ugccaugaaagcuuacauc

SEQ ID NO: 509 - RNA, 19 nt, Artificial Sequence
  Synthetic polynucleotide
uuagaagauggcuuauuga

SEQ ID NO: 510 - RNA, 19 nt, Artificial Sequence
  Synthetic polynucleotide
caaggccaagugggaugcc

SEQ ID NO: 511 - RNA, 19 nt, Artificial Sequence
  Synthetic polynucleotide
guauccguagugcuugucc

SEQ ID NO: 512 - RNA, 19 nt, Artificial Sequence
  Synthetic polynucleotide
ugaaccauauucugucuuc

SEQ ID NO: 513 - RNA, 19 nt, Artificial Sequence
  Synthetic polynucleotide
aaaguagaagagcuaaaga

SEQ ID NO: 514 - RNA, 19 nt, Artificial Sequence
  Synthetic polynucleotide
auguacuucaucacuaucu

SEQ ID NO: 515 - RNA, 19 nt, Artificial Sequence
  Synthetic polynucleotide
uuuuuggaacagucuuucc

SEQ ID NO: 516 - RNA, 19 nt, Artificial Sequence
  Synthetic polynucleotide
uuacacaacacuucgaugg

SEQ ID NO: 517 - RNA, 19 nt, Artificial Sequence
  Synthetic polynucleotide
aucaacaaaguagaagagc

SEQ ID NO: 518 - RNA, 19 nt, Artificial Sequence
  Synthetic polynucleotide
uaucucccgguugucguac

SEQ ID NO: 519 - RNA, 19 nt, Artificial Sequence
  Synthetic polynucleotide
aucccacuuggccuugccc

SEQ ID NO: 520 - RNA, 19 nt, Artificial Sequence
  Synthetic polynucleotide
aaacguuguaaacaugcuc

SEQ ID NO: 521 - RNA, 19 nt, Artificial Sequence
  Synthetic polynucleotide
uuuguagccaaauccuuuc

SEQ ID NO: 522 - RNA, 19 nt, Artificial Sequence
  Synthetic polynucleotide
augaaagcuuacaucaaca

SEQ ID NO: 523 - RNA, 19 nt, Artificial Sequence
  Synthetic polynucleotide
uugauaucaccacguaccu

SEQ ID NO: 524 - RNA, 19 nt, Artificial Sequence
  Synthetic polynucleotide
uucuugaggaggaaguagc

SEQ ID NO: 525 - RNA, 19 nt, Artificial Sequence
  Synthetic polynucleotide
ugauguaagcuuucauggc

SEQ ID NO: 526 - RNA, 19 nt, Artificial Sequence
  Synthetic polynucleotide
cuuggaagucccuuucagc

SEQ ID NO: 527 - RNA, 19 nt, Artificial Sequence
  Synthetic polynucleotide
aauaaguccagagcuuugg

SEQ ID NO: 528 - RNA, 19 nt, Artificial Sequence
  Synthetic polynucleotide
cuuucagcucauuccaggc

SEQ ID NO: 529 - RNA, 19 nt, Artificial Sequence
  Synthetic polynucleotide
caucccacuuggccuugcc

SEQ ID NO: 530 - RNA, 19 nt, Artificial Sequence
  Synthetic polynucleotide
uucgucgucaaacaaguug

SEQ ID NO: 531 - RNA, 19 nt, Artificial Sequence
  Synthetic polynucleotide
uucuacuucaauccucuug

SEQ ID NO: 532 - RNA, 19 nt, Artificial Sequence
  Synthetic polynucleotide
uacuuuguugauguaagcu

SEQ ID NO: 533 - RNA, 19 nt, Artificial Sequence
  Synthetic polynucleotide
aauagucacagauuguuug

SEQ ID NO: 534 - RNA, 19 nt, Artificial Sequence
  Synthetic polynucleotide
ucagcucauuccaggcauc

SEQ ID NO: 535 - RNA, 19 nt, Artificial Sequence
  Synthetic polynucleotide
ucaagguggaucagcuucc

SEQ ID NO: 536 - RNA, 19 nt, Artificial Sequence
  Synthetic polynucleotide
agcucauuccaggcauccc

SEQ ID NO: 537 - RNA, 19 nt, Artificial Sequence
  Synthetic polynucleotide
uucgaagucaucgucuuug

SEQ ID NO: 538 - RNA, 19 nt, Artificial Sequence
  Synthetic polynucleotide
uuggaaugaacacccuugc

SEQ ID NO: 539 - RNA, 19 nt, Artificial Sequence
  Synthetic polynucleotide
aagggacuuccaaggaaga

SEQ ID NO: 540 - RNA, 19 nt, Artificial Sequence
  Synthetic polynucleotide
uguugcagcuuccuacuuc

SEQ ID NO: 541 - RNA, 19 nt, Artificial Sequence
  Synthetic polynucleotide
uuucagcucauuccaggca

SEQ ID NO: 542 - RNA, 19 nt, Artificial Sequence
  Synthetic polynucleotide
uaugccaaaaccgaucuug

SEQ ID NO: 543 - RNA, 19 nt, Artificial Sequence
  Synthetic polynucleotide
gucccuuucagcucauucc

SEQ ID NO: 544 - RNA, 19 nt, Artificial Sequence
  Synthetic polynucleotide
uucgauuccacagugaucc

SEQ ID NO: 545 - RNA, 19 nt, Artificial Sequence
  Synthetic polynucleotide
uggaagucccuuucagcuc

SEQ ID NO: 546 - RNA, 19 nt, Artificial Sequence
  Synthetic polynucleotide
uaugucuucaccaacuaug

SEQ ID NO: 547 - RNA, 19 nt, Artificial Sequence
  Synthetic polynucleotide
uacaucaacaaaguagaag

SEQ ID NO: 548 - RNA, 19 nt, Artificial Sequence
  Synthetic polynucleotide
ucaagugucuccuaauaug

SEQ ID NO: 549 - RNA, 19 nt, Artificial Sequence
  Synthetic polynucleotide
uuggaagucccuuucagcu

SEQ ID NO: 550 - RNA, 19 nt, Artificial Sequence
  Synthetic polynucleotide
auuuuuucuuuagcucuuc

SEQ ID NO: 551 - RNA, 19 nt, Artificial Sequence
  Synthetic polynucleotide
uauuguuggauuucauguc

SEQ ID NO: 552 - RNA, 19 nt, Artificial Sequence
  Synthetic polynucleotide
aucuuccuuggaagucccu

SEQ ID NO: 553 - RNA, 19 nt, Artificial Sequence
  Synthetic polynucleotide
caucuuccuuggaaguccc

SEQ ID NO: 554 - RNA, 19 nt, Artificial Sequence
  Synthetic polynucleotide
uuuucuuuagcucuucuac

SEQ ID NO: 555 - RNA, 19 nt, Artificial Sequence
  Synthetic polynucleotide
auacgaucgaguuccuuga

SEQ ID NO: 556 - RNA, 19 nt, Artificial Sequence
  Synthetic polynucleotide
aaagcuuacaucaacaaag

SEQ ID NO: 557 - RNA, 19 nt, Artificial Sequence
  Synthetic polynucleotide
aaggccaagugggaugccu

SEQ ID NO: 558 - RNA, 19 nt, Artificial Sequence
  Synthetic polynucleotide
caaccgcgaaaaaguugcg

SEQ ID NO: 559 - RNA, 19 nt, Artificial Sequence
  Synthetic polynucleotide
ucccacuuggccuugcccg

SEQ ID NO: 560 - RNA, 19 nt, Artificial Sequence
  Synthetic polynucleotide
aaucauugcaggucagauc

SEQ ID NO: 561 - RNA, 19 nt, Artificial Sequence
  Synthetic polynucleotide
ucaaaucugcucucucugc

SEQ ID NO: 562 - RNA, 19 nt, Artificial Sequence
  Synthetic polynucleotide
uuuuuucuuuagcucuucu

SEQ ID NO: 563 - RNA, 19 nt, Artificial Sequence
  Synthetic polynucleotide
uauggaugauguucuuugc

SEQ ID NO: 564 - RNA, 19 nt, Artificial Sequence
  Synthetic polynucleotide
aggccaagugggaugccug

SEQ ID NO: 565 - RNA, 19 nt, Artificial Sequence
  Synthetic polynucleotide
aagugggaugccuggaaug

SEQ ID NO: 566 - RNA, 19 nt, Artificial Sequence
  Synthetic polynucleotide
uugauguaagcuuucaugg

SEQ ID NO: 567 - RNA, 19 nt, Artificial Sequence
  Synthetic polynucleotide
uucuuggccagcggugugg

SEQ ID NO: 568 - RNA, 19 nt, Artificial Sequence
  Synthetic polynucleotide
aguacaacugcaacaagug

SEQ ID NO: 569 - RNA, 19 nt, Artificial Sequence
  Synthetic polynucleotide
ugggaugccuggaaugagc

SEQ ID NO: 570 - RNA, 19 nt, Artificial Sequence
  Synthetic polynucleotide
gacuuccaaggaagaugcc

SEQ ID NO: 571 - RNA, 19 nt, Artificial Sequence
  Synthetic polynucleotide
cuggaaugagcugaaaggg

SEQ ID NO: 572 - RNA, 19 nt, Artificial Sequence
  Synthetic polynucleotide
ugaagaacgguaccagaug

SEQ ID NO: 573 - RNA, 19 nt, Artificial Sequence
  Synthetic polynucleotide
uaucaagccugccaauaug

SEQ ID NO: 574 - RNA, 19 nt, Artificial Sequence
  Synthetic polynucleotide
aaacagaucuuuacaagcu

SEQ ID NO: 575 - RNA, 19 nt, Artificial Sequence
  Synthetic polynucleotide
aucaguggacacuaugaca

SEQ ID NO: 576 - RNA, 19 nt, Artificial Sequence
  Synthetic polynucleotide
cugaaagggacuuccaagg

SEQ ID NO: 577 - RNA, 19 nt, Artificial Sequence
  Synthetic polynucleotide
uuacagugcgacagcuuga

SEQ ID NO: 578 - RNA, 19 nt, Artificial Sequence
  Synthetic polynucleotide
guugugaaugacguauugg

SEQ ID NO: 579 - RNA, 19 nt, Artificial Sequence
  Synthetic polynucleotide
ucuacuuuguugauguaag

SEQ ID NO: 580 - RNA, 19 nt, Artificial Sequence
  Synthetic polynucleotide
acguuguagaaguugugcc

SEQ ID NO: 581 - RNA, 19 nt, Artificial Sequence
  Synthetic polynucleotide
uccaggcaucccacuuggc

SEQ ID NO: 582 - RNA, 19 nt, Artificial Sequence
  Synthetic polynucleotide
cagcucauuccaggcaucc

SEQ ID NO: 583 - RNA, 19 nt, Artificial Sequence
  Synthetic polynucleotide
ugagauucgaggccuucug

SEQ ID NO: 584 - RNA, 19 nt, Artificial Sequence
  Synthetic polynucleotide
aauacaggaagucaauaug

SEQ ID NO: 585 - RNA, 19 nt, Artificial Sequence
  Synthetic polynucleotide
uaacaaugcugggaauuca

SEQ ID NO: 586 - RNA, 19 nt, Artificial Sequence
  Synthetic polynucleotide
uuccaggcaucccacuugg

SEQ ID NO: 587 - RNA, 19 nt, Artificial Sequence
  Synthetic polynucleotide
uuccuuggaagucccuuuc

SEQ ID NO: 588 - RNA, 19 nt, Artificial Sequence
  Synthetic polynucleotide
ucucauaggaguccaggug

SEQ ID NO: 589 - RNA, 19 nt, Artificial Sequence
  Synthetic polynucleotide
auuccaggcaucccacuug

SEQ ID NO: 590 - RNA, 19 nt, Artificial Sequence
  Synthetic polynucleotide
uagugcaucaccuguucgu

SEQ ID NO: 591 - RNA, 19 nt, Artificial Sequence
  Synthetic polynucleotide
gcacggaaguccaucugaa

SEQ ID NO: 592 - RNA, 19 nt, Artificial Sequence
  Synthetic polynucleotide
gcaggacaaagauguauua

SEQ ID NO: 593 - RNA, 19 nt, Artificial Sequence
  Synthetic polynucleotide
gggucuguggugauaaaua

SEQ ID NO: 594 - RNA, 19 nt, Artificial Sequence
  Synthetic polynucleotide
guaugagaacccaauguca

SEQ ID NO: 595 - RNA, 19 nt, Artificial Sequence
  Synthetic polynucleotide
gcaaagaccccaacgagaa

SEQ ID NO: 596 - RNA, 19 nt, Artificial Sequence
  Synthetic polynucleotide
auguuuacuaccaaaugga

SEQ ID NO: 597 - RNA, 19 nt, Artificial Sequence
  Synthetic polynucleotide
uaacugcagccaacuauuu

SEQ ID NO: 598 - RNA, 19 nt, Artificial Sequence
  Synthetic polynucleotide
uccaggaguuugucaauaa

SEQ ID NO: 599 - RNA, 19 nt, Artificial Sequence
  Synthetic polynucleotide
gcagauacuugagccauug

SEQ ID NO: 600 - RNA, 19 nt, Artificial Sequence
  Synthetic polynucleotide
ccggaaauuugaagaguau

SEQ ID NO: 601 - RNA, 17 nt, Artificial Sequence
  Synthetic polynucleotide
auguuuacuaccaaaug

SEQ ID NO: 602 - RNA, 17 nt, Artificial Sequence
  Synthetic polynucleotide
uccaggaguuugucaau

SEQ ID NO: 603 - RNA, 17 nt, Artificial Sequence
  Synthetic polynucleotide
gcagauacuugagccau

SEQ ID NO: 604 - RNA, 17 nt, Artificial Sequence
  Synthetic polynucleotide
uaacugcagccaacuau

SEQ ID NO: 605 - RNA, 17 nt, Artificial Sequence
  Synthetic polynucleotide
ccggaaauuugaagagu

SEQ ID NO: 606 - RNA, 17 nt, Artificial Sequence
  Synthetic polynucleotide
ggacauuuguguacuca

SEQ ID NO: 607 - RNA, 17 nt, Artificial Sequence
  Synthetic polynucleotide
uuuguggacgaaguacc

SEQ ID NO: 608 - RNA, 17 nt, Artificial Sequence
  Synthetic polynucleotide
uggccaaggucauccau

SEQ ID NO: 609 - RNA, 17 nt, Artificial Sequence
  Synthetic polynucleotide
gaaaggauuuggcuaca

SEQ ID NO: 610 - RNA, 19 nt, Artificial Sequence
  Synthetic polynucleotide
auguuuacuaccaaaugga

SEQ ID NO: 611 - RNA, 19 nt, Artificial Sequence
  Synthetic polynucleotide
uaacugcagccaacuauuu

SEQ ID NO: 612 - RNA, 19 nt, Artificial Sequence
  Synthetic polynucleotide
uccaggaguuugucaauaa

SEQ ID NO: 613 - RNA, 19 nt, Artificial Sequence
  Synthetic polynucleotide
gcagauacuugagccauug

SEQ ID NO: 614 - RNA, 19 nt, Artificial Sequence
  Synthetic polynucleotide
ccggaaauuugaagaguau

SEQ ID NO: 615 - RNA, 19 nt, Artificial Sequence
  Synthetic polynucleotide
ggacauuuguguacucacu

SEQ ID NO: 616 - RNA, 19 nt, Artificial Sequence
  Synthetic polynucleotide
uguuuguggacgaaguacc

SEQ ID NO: 617 - RNA, 19 nt, Artificial Sequence
  Synthetic polynucleotide
ccuggccaaggucauccau

SEQ ID NO: 618 - RNA, 19 nt, Artificial Sequence
  Synthetic polynucleotide
gagaaaggauuuggcuaca

SEQ ID NO: 619 - RNA, 19 nt, Artificial Sequence
  Synthetic polynucleotide
auguuuacuaccaaaugga

SEQ ID NO: 620 - RNA, 19 nt, Artificial Sequence
  Synthetic polynucleotide
uaacugcagccaacuauuu

SEQ ID NO: 621 - RNA, 19 nt, Artificial Sequence
  Synthetic polynucleotide
uccaggaguuugucaauaa

SEQ ID NO: 622 - RNA, 19 nt, Artificial Sequence
  Synthetic polynucleotide
gcagauacuugagccauug

SEQ ID NO: 623 - RNA, 19 nt, Artificial Sequence
  Synthetic polynucleotide
ccggaaauuugaagaguau

SEQ ID NO: 624 - RNA, 19 nt, Artificial Sequence
  Synthetic polynucleotide
ggacauuuguguacucacu

SEQ ID NO: 625 - RNA, 19 nt, Artificial Sequence
  Synthetic polynucleotide
gaaagcauauacagcaauu

SEQ ID NO: 626 - RNA, 19 nt, Artificial Sequence
  Synthetic polynucleotide
caaagaagauaaccaaugu

SEQ ID NO: 627 - RNA, 19 nt, Artificial Sequence
  Synthetic polynucleotide
gaaacauccuccggcugaa

SEQ ID NO: 628 - DNA, 64 bp, Artificial Sequence
  Synthetic polynucleotide
auuuucaagacuucauugacaguggccgauaagauagaagaucaaaaaaaggaacuagauggtt

SEQ ID NO: 629 - DNA, 59 bp, Artificial Sequence
  Synthetic polynucleotide
auuuucaagacuucauugacaguggccgauaagauagaagaucaaaaaaaggaacuatt

SEQ ID NO: 630 - DNA, 54 bp, Artificial Sequence
  Synthetic polynucleotide
auuuucaagacuucauugacaguggccgauaagauagaagaucaaaaaaaggtt

SEQ ID NO: 631 - DNA, 49 bp, Artificial Sequence
  Synthetic polynucleotide
auuuucaagacuucauugacaguggccgauaagauagaagaucaaaatt

SEQ ID NO: 632 - DNA, 44 bp, Artificial Sequence
  Synthetic polynucleotide
auuuucaagacuucauugacaguggccgauaagauagaagautt

SEQ ID NO: 633 - DNA, 39 bp, Artificial Sequence
  Synthetic polynucleotide
auuuucaagacuucauugacaguggccgauaagauagtt

SEQ ID NO: 634 - DNA, 34 bp, Artificial Sequence
  Synthetic polynucleotide
auuuucaagacuucauugacaguggccgauaatt

SEQ ID NO: 635 - DNA, 29 bp, Artificial Sequence
  Synthetic polynucleotide
auuuucaagacuucauugacaguggcctt

SEQ ID NO: 636 - DNA, 24 bp, Artificial Sequence
  Synthetic polynucleotide
auuuucaagacuucauugacagtt

SEQ ID NO: 637 - DNA, 21 bp, Artificial Sequence
  Synthetic polynucleotide
auuuucaagacuucauugatt

SEQ ID NO: 638 - RNA, 19 nt, Artificial Sequence
  Synthetic polynucleotide
uccaggaguuugucaauaa

SEQ ID NO: 639 - RNA, 19 nt, Artificial Sequence
  Synthetic polynucleotide
caaagacgaugacuucgaa

SEQ ID NO: 640 - RNA, 19 nt, Artificial Sequence
  Synthetic polynucleotide
ggacauuuguguacucacu

SEQ ID NO: 641 - RNA, 19 nt, Artificial Sequence
  Synthetic polynucleotide
gcuacuaucugauuuacug

SEQ ID NO: 642 - RNA, 19 nt, Artificial Sequence
  Synthetic polynucleotide
ggacauuuguguacucacu

SEQ ID NO: 643 - RNA, 19 nt, Artificial Sequence
  Synthetic polynucleotide
gcuacuaucugauuuacug

SEQ ID NO: 644 - RNA, 19 nt, Artificial Sequence
  Synthetic polynucleotide
ggacauuuguguacucacu

SEQ ID NO: 645 - RNA, 19 nt, Artificial Sequence
  Synthetic polynucleotide
gcuacuaucugauuuacug

SEQ ID NO: 646 - RNA, 19 nt, Artificial Sequence
  Synthetic polynucleotide
gaguuguguuuguggacga

SEQ ID NO: 647 - RNA, 19 nt, Artificial Sequence
  Synthetic polynucleotide
acucuagcgccaucgugcc

SEQ ID NO: 648 - DNA, 64 bp, Artificial Sequence; identical to SEQ ID NO: 628
  Synthetic polynucleotide

SEQ ID NO: 649 - DNA, 64 bp, Artificial Sequence
  Synthetic polynucleotide
ccaucuaguuccuuuuuuugaucuucuaucuuaucggccacugucaaugaagucuugaaaautt

SEQ ID NO: 650 - RNA, 19 nt, Artificial Sequence
  Synthetic polynucleotide
auguuuacuaccaaaugga

SEQ ID NO: 651 - RNA, 19 nt, Artificial Sequence
  Synthetic polynucleotide
uccaggaguuugucaauaa

SEQ ID NO: 652 - RNA, 19 nt, Artificial Sequence
  Synthetic polynucleotide
gcagauacuugagccauug

SEQ ID NO: 653 - RNA, 19 nt, Artificial Sequence
  Synthetic polynucleotide
uaacugcagccaacuauuu

SEQ ID NO: 654 - RNA, 19 nt, Artificial Sequence
  Synthetic polynucleotide
ccggaaauuugaagaguau

SEQ ID NO: 655 - RNA, 19 nt, Artificial Sequence
  Synthetic polynucleotide
ggacauuuguguacucacu

SEQ ID NO: 656 - RNA, 19 nt, Artificial Sequence
  Synthetic polynucleotide
uguuuguggacgaaguacc

SEQ ID NO: 657 - RNA, 19 nt, Artificial Sequence
  Synthetic polynucleotide
ccuggccaaggucauccau

SEQ ID NO: 658 - RNA, 19 nt, Artificial Sequence
  Synthetic polynucleotide
gagaaaggauuuggcuaca